(19) United States
(12) Patent Application Publication
(10) Pub. No.: US 2026/0217724 A1
(43) Pub. Date: Jul. 30, 2026

(54) SALTS AND CRYSTALLINE FORMS OF AN AP2 ASSOCIATED KINASE 1 INHIBITOR

(71) Applicant: Satellos Bioscience Inc., Toronto (CA)

(72) Inventors: James Brian JAQUITH, Cobourg (CA); Philip D. LAMBERT, Annapolis, MD (US)

(21) Appl. No.: 19/431,018

(22) Filed: Dec. 23, 2025

Related U.S. Application Data
(60) Provisional application No. 63/738,369, filed on Dec. 23, 2024.

(51) Int. Cl. C07D 487/04; A61K 31/194; A61K 31/519; C07C 55/06

(52) U.S. Cl. CPC C07D 487/04; A61K 31/194; A61K 31/519; C07C 55/06; C07B 2200/13

(57) ABSTRACT

The present disclosure relates to pharmaceutically acceptable salts and crystalline forms of Compound 1, pharmaceutical compositions comprising the salts and crystalline forms of Compound 1, and their use in treating muscular diseases, such as Duchene Muscular Dystrophy (DMD), that are regulated by AAK1.

### CROSS-REFERENCE TO RELATED APPLICATIONS

The present application claims the benefit of priority to U.S. Provisional Application No. 63/738,369, filed Dec. 23, 2024, which is incorporated herein by reference in its entirety.

### FIELD OF THE INVENTION

The present disclosure relates to Compound 1 salts, crystalline forms of Compound 1 or salts thereof, and polymorph forms of Compound 1, as well as pharmaceutical compositions comprising Compound 1 salts and crystalline forms of Compound 1, and their use in treating muscular diseases mediated by AAK1 activity, such as Duchenne Muscular Dystrophy. The crystalline forms disclosed herein can be of the Compound 1 free base or a pharmaceutically acceptable salt of Compound 1, or a cocrystal of Compound 1. The present disclosure also describes amorphous and co-amorphous forms of Compound 1.

### BACKGROUND OF THE INVENTION

Compound 1 is an AP2 Associated Kinase 1 (AAK1) inhibitor having the structure:

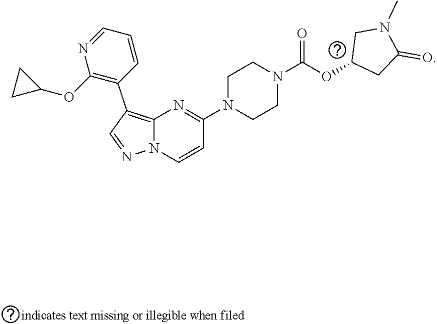 [SMILES: CN1C[C@@H](OC(=O)N2CCN(c3ccn4ncc(-c5cccnc5OC5CC5)c4n3)CC2)CC1=O]

Compound 1 is being studied as a treatment for muscular diseases and disorders, including Duchenne muscular dystrophy (DMD).

To facilitate these studies and further development, there is a need for isolable and stable forms of Compound 1 and processes for making the same.

### SUMMARY OF THE INVENTION

The present disclosure provides pharmaceutically acceptable salts and crystalline forms of Compound 1. In some embodiments, the crystalline form of Compound 1 is a pharmaceutically acceptable salt of Compound 1. In some embodiments, the crystalline form of Compound 1 is a cocrystal of Compound 1. In some embodiments, the crystalline form of Compound 1 is the freebase of Compound 1. In some embodiments, the crystalline forms of Compound 1 are polymorphs.

The present disclosure also provides pharmaceutical compositions comprising the pharmaceutically acceptable salts and crystalline forms of Compound 1 provided herein.

The present disclosure further provides methods of making the pharmaceutically acceptable salts and crystalline forms of Compound 1.

In some embodiments, the present disclosure provides an oxalate, a bromide, a chloride, or a phosphate salt of Compound 1. In some embodiments, the oxalate, the bromide, the chloride, or the phosphate salt of Compound 1 is crystalline.

In some embodiments, the present disclosure provides a crystalline form of Compound 1 that is an oxalate salt of Compound 1. In some embodiments, the present disclosure provides a crystalline form of Compound 1 that is an oxalic acid cocrystal of Compound 1. The oxalic acid cocrystal of Compound 1 may be referred to herein as Compound 1 Form I. In some embodiments, the present disclosure provides polymorph forms of the Compound 1 oxalate salt. In some embodiments, the present disclosure provides polymorph forms of the Compound 1 oxalic acid cocrystal.

In some embodiments, the present disclosure provides an oxalic acid co-amorphous form of Compound 1 (“Compound 1 oxalic acid co-amorphous form”).

In some embodiments, the present disclosure provides a crystalline form of Compound 1 that is a bromide salt of Compound 1. In some embodiments, the present disclosure provides a crystalline form of Compound 1 that is hydrobromide cocrystal of Compound 1. The crystalline bromide salt of Compound 1 and the hydrobromide cocrystal of Compound 1 may be referred to herein as Compound 1 Form II. In some embodiments, the present disclosure provides polymorph forms of the Compound 1 bromide salt. In some embodiments, the present disclosure provides polymorph forms of the Compound 1 hydrobromide cocrystal.

In some embodiments, the present disclosure provides a hydrobromide co-amorphous form of Compound 1 (“Compound 1 hydrobromide co-amorphous form”).

In some embodiments, the present disclosure provides a crystalline form of Compound 1 that is a chloride salt of Compound 1. In some embodiments, the present disclosure provides a crystalline form of Compound 1 that is hydrochloride cocrystal of Compound 1. The crystalline chloride salt of Compound 1 and the hydrochloride cocrystal of Compound 1 may be referred to herein as Compound 1 Form III. In some embodiments, the present disclosure provides polymorph forms of the Compound 1 chloride salt. In some embodiments, the present disclosure provides polymorph forms of the Compound 1 hydrochloride cocrystal.

In some embodiments, the present disclosure provides a hydrochloride co-amorphous form of Compound 1 (“Compound 1 hydrochloride co-amorphous form”).

In some embodiments, the present disclosure provides a crystalline form of Compound 1 that is a phosphate salt of Compound 1. In some embodiments, the present disclosure provides a crystalline form of Compound 1 that is phosphoric acid cocrystal of Compound 1. The crystalline phosphate salt of Compound 1 and the phosphoric acid cocrystal of Compound 1 may be referred to herein as Compound 1 Form IV. In some embodiments, the present disclosure provides polymorph forms of the Compound 1 phosphate salt. In some embodiments, the present disclosure provides polymorph forms of the Compound 1 phosphoric acid cocrystal.

In some embodiments, the present disclosure provides a phosphoric acid co-amorphous form of Compound 1 (“Compound 1 phosphoric acid co-amorphous form”).

In some embodiments, the present disclosure provides crystalline freebase forms of Compound 1 (“Compound 1 Freebase”). In some embodiments, the present disclosure provides polymorphic freebase forms of Compound 1. In one embodiment, the crystalline form is Compound 1 Form A freebase. In one embodiment, the crystalline form is Compound 1 Form B freebase. In one embodiment, the crystalline form is Compound 1 Form C freebase. In one embodiment, the crystalline form is Compound 1 Form D freebase. In one embodiment, the crystalline form is Compound 1 Form E freebase. In one embodiment, the crystalline form is Compound 1 Form F freebase. In one embodiment, the crystalline form is Compound 1 Form G freebase. In one embodiment, the crystalline form is Compound 1 Form H freebase. In one embodiment, the crystalline form is Compound 1 Form I freebase. In one embodiment, the crystalline form is Compound 1 Form J freebase.

In some embodiments, the present disclosure provides an amorphous form of Compound 1. In some embodiments, the amorphous form is a co-amorphous form comprising Compound 1 and a coformer. In some embodiments, the coformer is a Bronsted acid, including, but not limited to, oxalic acid, hydrobromic acid, hydrochloric acid, and phosphoric acid.

### BRIEF DESCRIPTION OF THE DRAWINGS

FIG. 1A shows an X-ray power diffraction (XRPD) spectrum for Compound 1 Form I. FIG. 1B shows differential scanning calorimetry (DSC) thermogram of Compound 1 Form I. FIG. 1C shows a thermogravimetric analysis (TGA) thermogram of Compound 1 Form I. FIG. 1D provides dynamic vapor sorption (DVS) plot showing the change in mass over time (left panel) and a DVS isotherm plot showing the reversibility of moisture uptake. FIG. 1E shows a comparison of XRPD spectra pre- and post-DVS analysis. FIG. 1F provides polarized light microscopy (PLM) images of Compound 1 Form I produced according to the procedures of Example 1 (100 mg scale). FIG. 1G provides polarized light microscopy (PLM) images of Compound 1 Form I produced according to the procedures of Example 1 (1.5 g scale). FIG. 1H provides a scanning electron microscopy image of Compound 1 Form I. FIG. 1I provides an FTIR spectrum of the Compound 1 Form I. FIG. 1J provides a single x-ray crystal structure of the Compound 1 Form I. FIG. 1K provides an overlay of Compound 1 Form I units cells showing hydrogen-bond interactions between O6-(H)—N2 and O5-(H)—O4. FIG. 1L provides ¹H NMR spectrum in DMSO-d⁶ for the Compound 1 Form I. FIG. 1M provides a chromatogram showing the oxalic acid content in the Compound 1 Form I.

FIG. 2A shows an X-ray power diffraction (XRPD) spectrum for Compound 1 Form II. FIG. 2B shows differential scanning calorimetry (DSC) thermogram of Compound 1 Form II. FIG. 2C shows a thermogravimetric analysis (TGA) thermogram of Compound 1 Form II. FIG. 2D provides dynamic vapor sorption (DVS) plot showing the change in mass over time (left panel) and a DVS isotherm plot showing the reversibility of moisture uptake. FIG. 2E shows a comparison of XRPD spectra pre- and post-DVS analysis. FIG. 2F provides polarized light microscopy (PLM) images of Compound 1 Form II produced according to the procedures of Example 2 (100 mg scale). FIG. 2G provides polarized light microscopy (PLM) images of Compound 1 Form II produced according to the procedures of Example 2 (1.5 g scale). FIG. 2H provides a scanning electron microscopy image of Compound 1 Form II. FIG. 2I provides an FTIR spectrum of the Compound 1 Form II.

FIG. 3A shows an X-ray power diffraction (XRPD) spectrum for Compound 1 Form III. FIG. 3B shows differential scanning calorimetry (DSC) thermogram of Compound 1 Form III. FIG. 3C shows a thermogravimetric analysis (TGA) thermogram of Compound 1 Form III. FIG. 3D provides dynamic vapor sorption (DVS) plot showing the change in mass over time (left panel) and a DVS isotherm plot showing the reversibility of moisture uptake. FIG. 3E shows a comparison of XRPD spectra pre- and post-DVS analysis. FIG. 3F provides polarized light microscopy (PLM) images of Compound 1 Form III produced according to the procedures of Example 3 (100 mg scale). FIG. 3G provides polarized light microscopy (PLM) images of Compound 1 Form III produced according to the procedures of Example 3 (1.5 g scale). FIG. 3H provides a scanning electron microscopy image of Compound 1 Form III. FIG. 3I provides an FTIR spectrum of the Compound 1 Form III.

FIG. 4A shows an X-ray power diffraction (XRPD) spectrum for Compound 1 Form IV. FIG. 4B shows differential scanning calorimetry (DSC) thermogram of Compound 1 Form IV. FIG. 4C shows a thermogravimetric analysis (TGA) thermogram of Compound 1 Form IV. FIG. 4D provides dynamic vapor sorption (DVS) plot showing the change in mass over time (left panel) and a DVS isotherm plot showing the reversibility of moisture uptake. FIG. 4E shows a comparison of XRPD spectra pre- and post-DVS analysis. FIG. 4F provides polarized light microscopy (PLM) images of Compound 1 Form IV produced according to the procedures of Example 4 (100 mg scale). FIG. 4G provides polarized light microscopy (PLM) images of Compound 1 Form IV produced according to the procedures of Example 4 (1.5 g scale). FIG. 4H provides a scanning electron microscopy image of Compound 1 Form IV. FIG. 4I provides an FTIR spectrum of the Compound 1 Form IV.

FIG. 5A shows an X-ray power diffraction (XRPD) spectrum for Compound 1 crystalline Form A freebase. FIG. 5B shows differential scanning calorimetry (DSC) thermogram of Compound 1 crystalline Form A freebase. FIG. 5C shows a thermogravimetric analysis (TGA) thermogram of Compound 1 crystalline Form A freebase. FIG. 5D provides dynamic vapor sorption (DVS) plot showing the change in mass over time (left panel) and a DVS isotherm plot showing the reversibility of moisture uptake. FIG. 5E shows a comparison of XRPD spectra pre- and post-DVS analysis. FIG. 5F provides polarized light microscopy (PLM) images of Compound 1 crystalline Form A freebase produced according to the procedure of Example 5 (300 mg scale). FIG. 5G provides a scanning electron microscopy image of Compound 1 crystalline Form A freebase. FIG. 5H provides an FTIR spectrum of the Compound 1 crystalline Form A freebase.

FIG. 6A shows an X-ray power diffraction (XRPD) spectrum for Compound 1 crystalline Form C freebase. FIG. 6B shows differential scanning calorimetry (DSC) thermogram of Compound 1 crystalline Form C freebase. FIG. 6C shows a thermogravimetric analysis (TGA) thermogram of Compound 1 crystalline Form C freebase. FIG. 6D provides dynamic vapor sorption (DVS) plot showing the change in mass over time (left panel) and a DVS isotherm plot showing the reversibility of moisture uptake. FIG. 6E shows a comparison of XRPD spectra pre- and post-DVS analysis. FIG. 6F provides polarized light microscopy (PLM) images of Compound 1 crystalline Form C freebase produced according to the procedure of Example 6 (80 mg scale). FIG. 6G provides polarized light microscopy (PLM) images of Compound 1 crystalline Form C freebase produced according to the procedure of Example 6 (500 mg scale). FIG. 6H provides a scanning electron microscopy image of Compound 1 crystalline Form C freebase. FIG. 6I provides an FTIR spectrum of the Compound 1 crystalline Form C freebase.

FIG. 7A provides the XRPD diffractograms from the polymorph slurry plate screening results from the solvent combinations of Table 3. FIG. 7B provides the XRPD diffractograms from the polymorph cooling plate screening results from the solvents of Table 3. FIG. 7C provides the XRPD diffractograms from the polymorph antisolvent plate screening results for the solvents of Table 4.

FIG. 8 provides a summary of the polymorphic landscape of Compound 1, including crystallization conditions and characteristics of the isolated forms.

FIG. 9 provides a comparison of XRPD diffractograms for Compound 1 Forms A-J.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the term “about” may be used in conjunction with numerical values and/or ranges. As used herein, the word “about” when immediately preceding a stated numerical value means±10% of the numerical value. By way of example, “about 100 mg/kg” means 90 mg/kg to 110 mg/kg. Unless expressly stated to the contrary, all ranges cited herein include their upper and lower endpoints and all values therebetween. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a compound” may refer to one or more compounds or at least one compound. As such, the terms “a” (or “an”), “one or more” and “at least one” are used interchangeably herein. In addition, reference to “a compound” by the indefinite article “a” or “an” does not exclude the possibility that more than one of the compounds is present, unless the context clearly requires that there is one and only one of the inhibitors.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The present disclosure provides crystalline forms of Compound 1, e.g., pharmaceutically acceptable salts, cocrystals, or a freebase of Compound 1. The crystalline forms may exhibit polymorphism. Polymorphism can be characterized as the ability of a compound to crystallize into different crystal forms while maintaining the same structural formula (i.e., the covalent bonds in the compound are the same in different crystal forms). A crystalline polymorph of a given drug substance is chemically identical to any other crystalline polymorph of that drug substance in containing the same atoms bonded to one another in the same way, but differs in its crystal forms, which can affect one or more physical properties, such as stability, solubility, melting point, bulk density, flow properties, etc., or pharmacological properties such as bioavailability, etc.

The crystalline forms disclosed herein can be characterized by the interlattice plane intervals determined by an X-ray powder diffraction pattern (XRPD). The XRPD diffractogram is typically represented by a diagram plotting the intensity of the peaks versus the location of the peaks, i.e., diffraction angle 2 θ (two-theta) in degrees. Characteristic peaks of a given XRPD diffractogram can be selected according to the peak locations and their relative intensity to conveniently distinguish this crystalline structure from others. The % intensity of the peaks relative to the most intense peak may be represented as I/Io.

Those skilled in the art recognize that the measurements of the XRPD peak locations and/or intensity for a given crystalline form of the same compound will vary within a margin of error. The values of degree 2θ allow for appropriate error margins. Typically, the error margins are represented by “±”. For example, the degree 20 of “8.716±0.3” denotes a range from 8.716+0.3, i.e., 9.016, to 8.716-0.3, i.e., 8.416. Depending on the sample preparation technique, the calibration technique applied to the instrument, human operational variation, and etc., those skilled in the art recognize that the appropriate error of margins for an XRPD can be ±0.7; ±0.6; ±0.5; ±0.4; ±0.3; ±0.2; ±0.1; ±0.05; or less. Additional details of the methods and equipment used for the XRPD analysis are described in the Examples section.

Differential Scanning Calorimetry (DSC) can also be used to characterize the crystalline forms. The DSC thermogram is typically expressed by a diagram plotting the normalized heat flow in units of Watts/gram (“W/g”) versus the measured sample temperature in degree C. The DSC thermogram is usually evaluated for extrapolated onset and end (outset) temperatures, peak temperature, and heat of fusion. A peak characteristic value of a DSC thermogram is often used as the characteristic peak to distinguish this crystalline structure from others.

Those skilled in the art recognize that the measurements of the DSC thermogram for a given crystalline form of the same compound will vary within a margin of error. The values of a single peak characteristic value, expressed in degree C., allow appropriate error margins. Typically, the error margins are represented by “±”. For example, the single peak characteristic value of t “53.09±2.0” denotes a range from 53.09±2, i.e., 55.09, 53.09-2, i.e., 51.09. Depending on the sample preparation techniques, the calibration techniques applied to the instruments, human operational variations, and etc., those skilled in the art recognize that the appropriate error of margins for a single peak characteristic value can be ±5.0; ±4.0; ±3.0; ±2.0; ±1.0; or less. Additional details of the methods and equipment used for the DSC thermogram analysis are described in the Examples section.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

In embodiments, a cocrystal of Compound 1 as described herein comprises Compound 1 and at least one additional chemical species, generally referred to as a “cocrystal former” or “coformer.” As used herein, the term “cocrystal” refers to a crystalline solid comprising Compound 1 and a coformer in the same crystal lattice, which has a defined stoichiometric ratio and possesses distinct physical, crystallographic and spectroscopic properties when compared to the chemical species individually. The coformer may be H-bonded directly to Compound 1 or may be H-bonded to an additional molecule (a second coformer) which is H-bonded to Compound 1. In either case, a non-covalent bond is formed between a hydrogen bond donor of one of the chemical species and a hydrogen bond acceptor of the other (i.e., Compound 1). The coformer of the present disclosure is generally a Bronsted acid including, but not limited to, oxalic acid, hydrobromic acid, hydrochloric acid, and phosphoric acid. The coformer can also be a water and/or solvent molecule. In particular embodiments, the coformer comprises a pharmaceutically acceptable acid, e.g., oxalic acid, hydrobromic acid, hydrochloric acid, and phosphoric acid.

A “Compound 1 cocrystal” as used herein is distinct from a “Compound 1 salt,” which comprises charged-balanced charged species. The species making up a cocrystal typically are neutral, and are generally held together by weak, reversible, non-covalent interactions. The weak interaction generally includes, e.g., hydrogen bonding, van der Waals forces, π-π interactions, and/or halogen bond interactions. Cocrystals can therefore be distinguished from salt that has an ionic bond framework by the absence of a proton transfer between the chemical species (e.g., Compound 1 and a Bronsted acid).

As used herein, “solvate” refers to a complex formed by solvation that results from the combination of solvent molecules, e.g., acetonitrile, water, etc., with molecules or ions of the active agent disclosed herein, or an aggregate that consists of a solute ion or molecule of the active agent disclosed herein with one or more solvent molecules. In some embodiments, the solvate is a hydrate. The term “hydrate”, as used herein, refers to a solvate of which the solvent is water. Examples of a hydrate include, but are not limited to, hemihydrate, monohydrate, dihydrate, trihydrate, hexahydrate, etc. The solvate can be formed via solvation or hydration that is either part of the preparation of the present compound or through natural absorption of moisture by the anhydrous (i.e., water-free) compound of the present disclosure. The solvates disclosed herein may exist, for example, with two, three, four, five, or six Compound 1 salt molecules per solvate or per hydrate molecule. Pharmaceutically acceptable solvents used for crystallization, such as alcohols (e.g., methanol and ethanol); aldehydes; ketones, (e.g., acetone); and esters, e.g., ethyl acetate, may be embedded in the crystal grating or lattice. The identification of a solvate can be achieved by any method known in the art, including nuclear magnetic resonance (NMR) spectroscopy. In some embodiments, the Compound 1 salts disclosed herein are non-solvates, meaning the crystal grating or lattice is free (per the limits of detection) of solvent and water molecules.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

An “effective amount” means the amount of a compound or formulation according to the disclosure that, when administered to a patient for treating a state, disorder or condition is sufficient to effect such treatment. The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. “Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an XRPD pattern that is “substantially” similar to another XRPD pattern would mean that the one skilled in the art would understand the two patterns to be of the same substance in the same form. The exact allowable degree of deviation from absolute completeness may in some cases depend on the specific context. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

Polymorphism can be characterized as the ability of a compound to crystallize into different crystal forms, while maintaining the same chemical formula. A crystalline polymorph of a given drug substance is chemically identical to any other crystalline polymorph of that drug substance in containing the same atoms bonded to one another in the same way, but differs in its crystal forms, which can affect one or more physical properties, such as stability, solubility, melting point, bulk density, flow properties, bioavailability, etc.

In some embodiments, the crystalline form of Compound 1 disclosed herein comprises a mixture of polymorph forms (e.g., a 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1 mixture, or greater). In some embodiments, the crystalline form of Compound 1 disclosed herein is a single polymorph form. In some embodiments, the crystalline form is substantially pure, e.g., comprising over about 99.9%, about 99.8%, about 99.7%, about 99.6%, about 99.5%, about 99.4%, about 99.3%, about 99.2%, about 99.1%, or about 99.0% of a crystalline form. In another embodiment, the crystalline form of Compound 1 comprises greater than about 99%, 98%, 97%, 96%, 95%, 94%, 93%, 92%, 91%, or 90% of a single form. In some embodiments, the crystalline form of Compound 1 comprises greater than about 90%, 85%, 80%, 75%, 70%, 65%, 60%, 55%, 50%, 45%, or 40% of a single form.

In one embodiment, the crystalline form of Compound 1 disclosed herein is isolated and purified. In one embodiment, the purified form does not comprise any detectable residual solvent by ¹H NMR. In one embodiment, the purified form is at least 95%, at least 96%, at least 97%, at least 98%, at least 99%, or at least 99.5% pure.

### Compound 1

Compound 1 is an AP2 Associated Kinase 1 (AAK1) inhibitor having the structure:

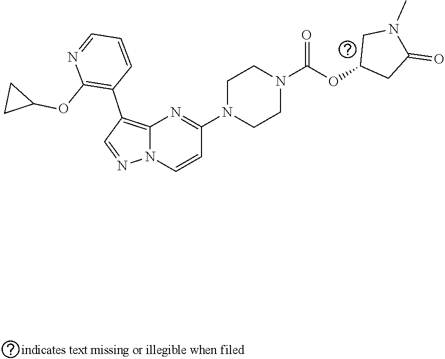 [SMILES: CN1C[C@@H](OC(=O)N2CCN(c3ccn4ncc(-c5cccnc5OC5CC5)c4n3)CC2)CC1=O]

The synthesis and therapeutic uses of Compound 1 are described in International Application No. US2024/045579, which is incorporated herein by reference in its entirety for all purposes.

The present disclosure provides pharmaceutically acceptable salts, crystalline forms, and polymorph forms of Compound 1. The present disclosure also provides amorphous forms of Compound 1 and pharmaceutically salts thereof. Methods of treatment and methods of preparation of the disclosed compounds are also provided herein.

### Compound 1 Form I

In some embodiments, the present disclosure provides an oxalate salt or oxalic acid cocrystal of Compound 1. In some embodiments, the oxalate salt of Compound 1 is crystalline. This disclosure encompasses all oxalate anions and all charge variants of Compound 1. Applicant surprisingly discovered that the oxalate salt is not hygroscopic. In some embodiments, the oxalic acid cocrystal of Compound 1 is Compound 1 Form I. In some embodiments, the crystalline form is a polymorph form. The oxalate salt or oxalic acid cocrystal can have a ratio of Compound 1:oxalic acid of about 1:1, about 1:2, or about 1:3, as exemplified by the structure below. In some embodiments, the ratio of Compound 1:oxalic acid in the salt or cocrystal is about 1:1. Applicant surprisingly discovered that the oxalic acid cocrystal of Compound 1 is stable and does not undergo a form change to a different crystalline form.

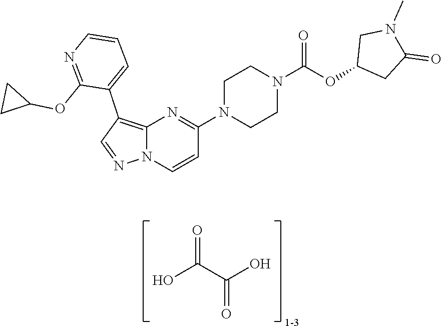

In one embodiment, the present disclosure provides Compound 1 Form I. In some embodiments, the Compound 1 Form I exhibits an XRPD pattern comprising 1, 2, 3, 4, or 5 or more peaks at 6.68, 8.43, 10.29, 10.82, 12.28, 14.37, 20.60, 21.78, and 22.45 degrees two-theta with the margin of error of 0.5; ±0.4; ±0.3; ±0.2; ±0.1; ±0.05; or less. In some embodiments, the Compound 1 Form I exhibits an XRPD pattern comprising peaks at 6.68, 8.43, 10.29, 10.82, 12.28, 14.37, 20.60, 21.78, and 22.45 degrees two-theta with the margin of error of 0.5; ±0.4; ±0.3; ±0.2; ±0.1; ±0.05; or less. In some embodiments, the Compound 1 Form I exhibits an XRPD pattern comprising peaks at 10.29, 12.28, and 21.78 degrees two-theta with the margin of error of 0.5; ±0.4; ±0.3; ±0.2; ±0.1; ±0.05; or less. In some embodiments, the XRPD peaks have a margin of error of ±0.2. In some embodiments, the Compound 1 Form I exhibits an XRPD pattern comprising peaks at 10.29±0.2, 12.28±0.2, and 21.78±0.2 degrees two-theta. In some embodiments, the XRPD peaks have a margin of error of 0.1. In yet another embodiment, the Compound 1 Form I exhibits an XRPD comprising peaks shown in Table 1 below.

TABLE 1
XRPD Table of Compound 1 Form I

| | Relative |
|---|---|
| 2-Theta | Intensity % |
| 6.68 | 10.1 |
| 8.43 | 17.9 |
| 10.29 | 100 |
| 10.82 | 16.6 |
| 12.28 | 79.0 |
| 14.37 | 10.7 |
| 20.60 | 11.0 |
| 21.78 | 25.4 |
| 22.45 | 11.6 |

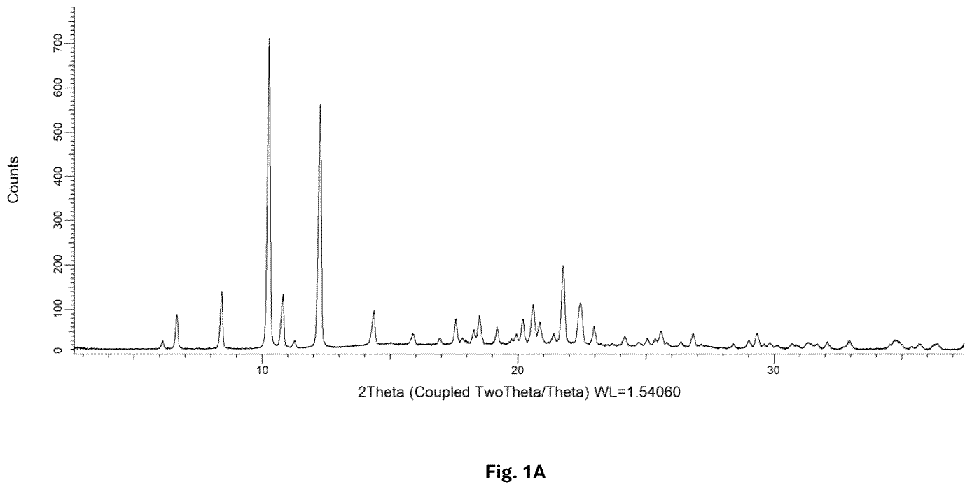

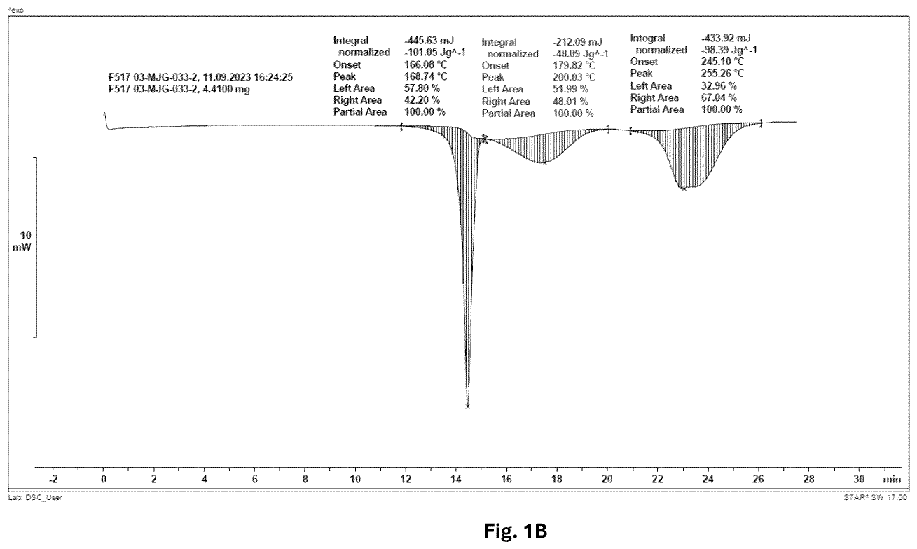

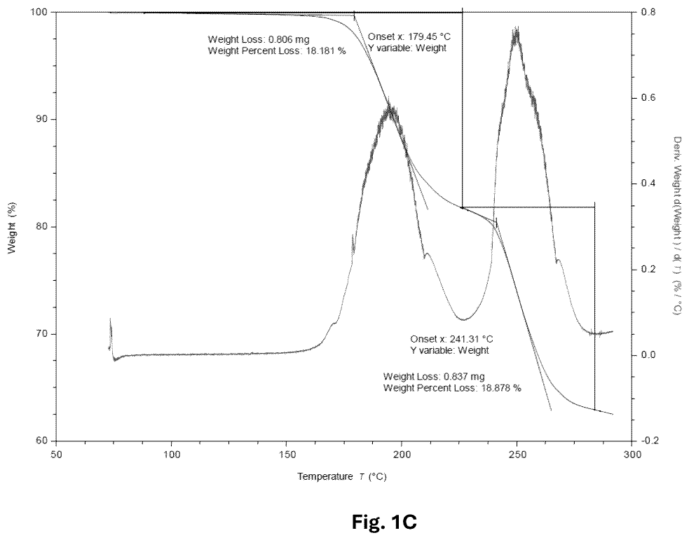

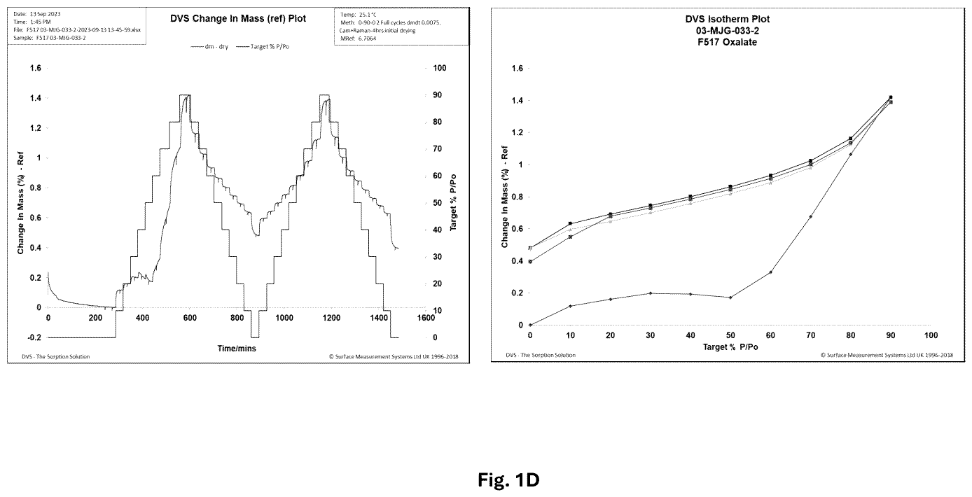

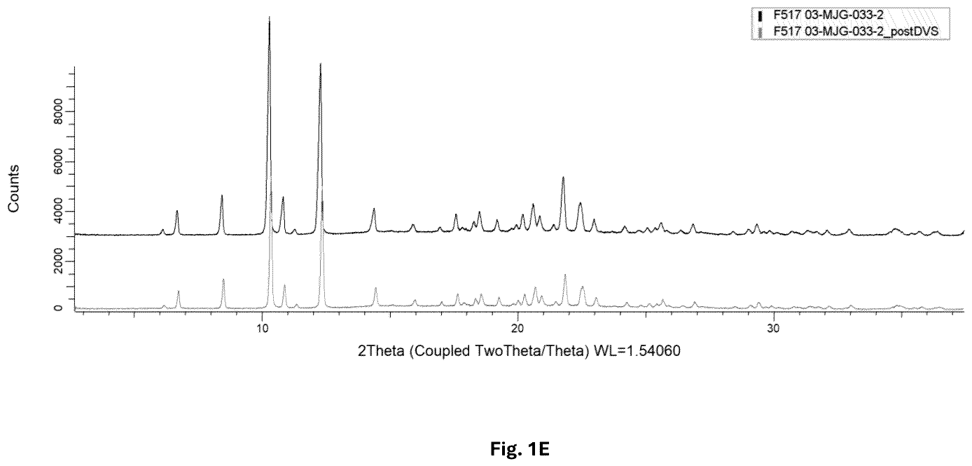

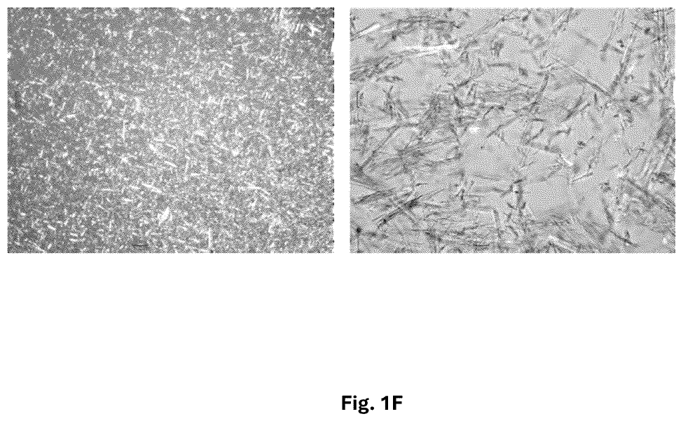

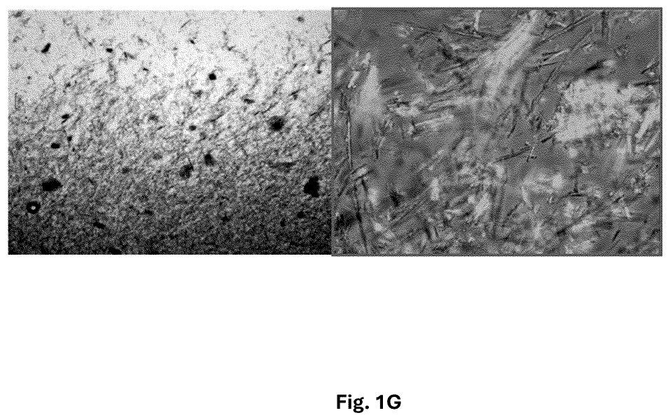

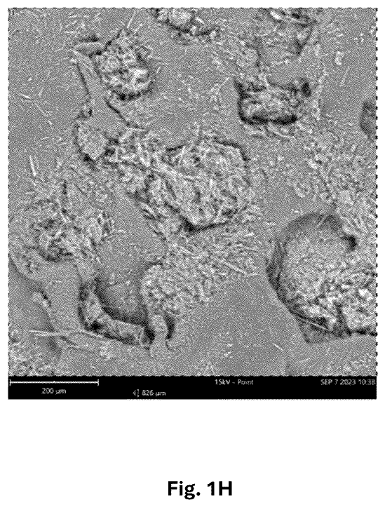

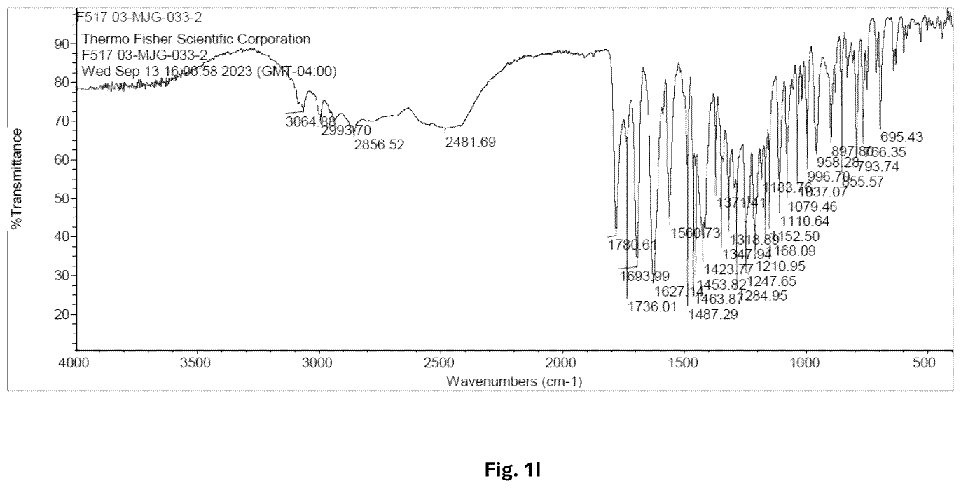

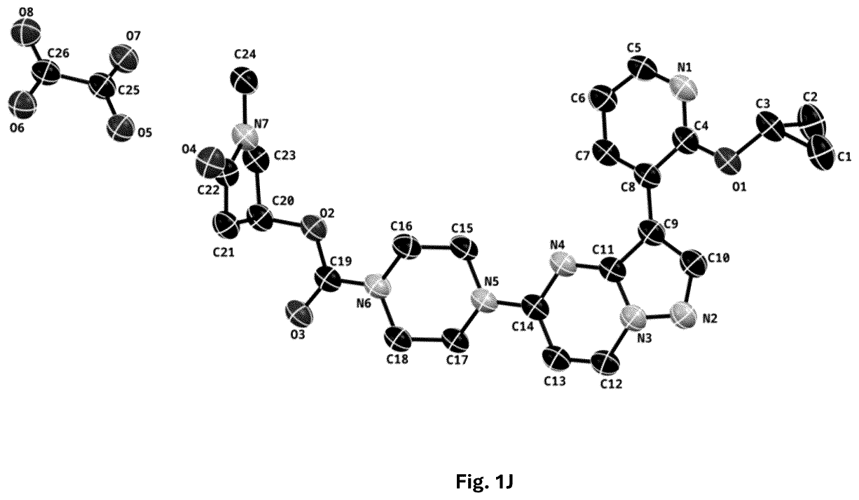

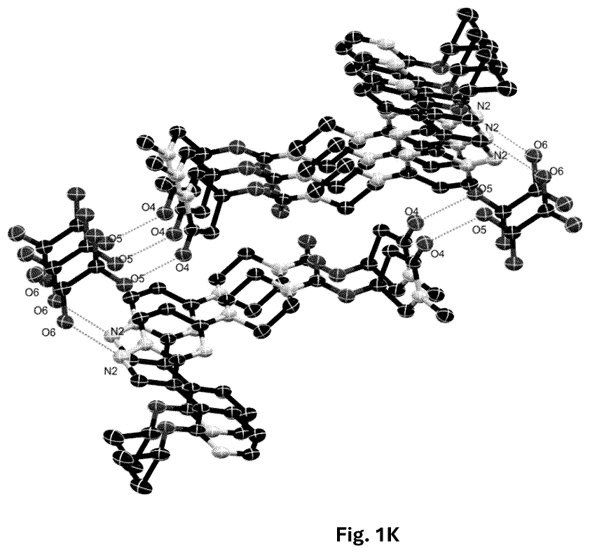

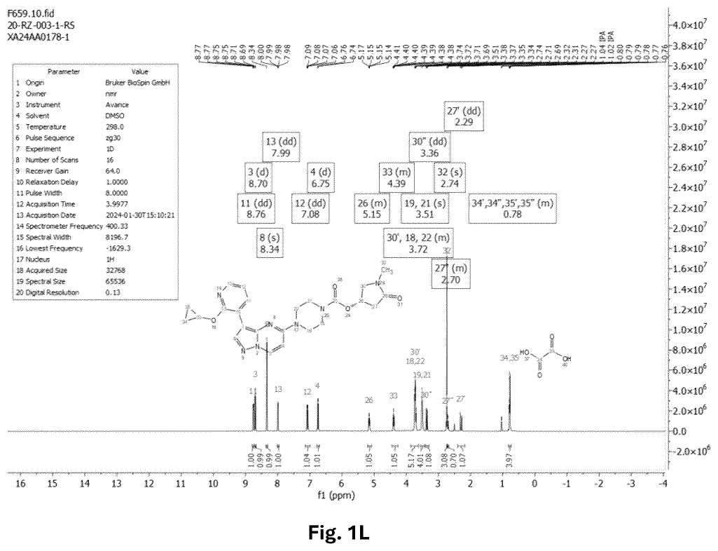

In some embodiments, the Compound 1 Form I exhibits an XRPD that is substantially similar to FIG. 1A.

In some embodiments, the Compound 1 Form I exhibits a DSC thermogram comprising an initial sharp endotherm at 169° C. with the margin of error of 10.0; ±5.0; ±4.0; 3.0; 2.0; 1.0; or less. In some embodiments, the Compound 1 Form I exhibits a DSC thermogram comprising two additional endotherms with onset temperatures of 180° C. and 245° C. with the margin of error of ±10.0; ±5.0; ±4.0; ±3.0; ±2.0; ±1.0; or less. In some embodiments, the Compound 1 Form I exhibits a DSC thermogram that is substantially similar to FIG. 1B.

In some embodiments, the Compound 1 Form I exhibits a TGA thermogram that is substantially similar to FIG. 1C. In some embodiments, the TGA thermogram of the Compound 1 Form I exhibits a weight loss of about 15-25% that onsets at a temperature of 179±5° C. In other embodiments, the TGA thermogram of the Compound 1 Form I exhibits a weight loss of about 18.2% that onsets at a temperature of about 179° C. In some embodiments, the TGA thermogram of the Compound 1 Form I exhibits a second weight loss of about 15-25% that onsets at a temperature of 241±5° C. In further embodiments, the TGA thermogram of the Compound 1 Form I exhibits a second weight loss of about 18.9% that onsets at a temperature of about 241° C.

In some embodiments, the Compound 1 Form I exhibits a DVS isotherm plot that is substantially similar to FIG. 1D. In some embodiments, the Compound 1 Form I exhibits a mass increase of about 1-5% due to moisture uptake at 80-90% relative humidity (RH). In some embodiments, the Compound 1 Form I exhibits a mass increase of about 1-2% due to moisture uptake at 80-90% relative humidity (RH). In some embodiments, the Compound 1 Form I exhibits a mass increase of about 1.4% due to moisture uptake at about 90% RH. In some embodiments, the Compound 1 Form I exhibits a mass increase of less than 0.5% due to moisture uptake at 50-60% RH. In some embodiments, the Compound 1 Form I exhibits a mass increase of about 0.1-0.5% due to moisture uptake at about 50% RH. In some embodiments, the Compound 1 Form I exhibits a mass increase of about 0.2% due to moisture uptake at about 50% RH. In some embodiments, the Compound 1 Form I exhibits a mass loss on drying of about 0.5% or less at about 0% RH. In some embodiments, the Compound 1 Form I exhibits a mass loss on drying of about 0.1-0.5% at about 0% RH. In some embodiments, the Compound 1 Form I exhibits a mass loss on drying of about 0.2% at about 0% RH.

The Compound 1 salts and cocrystals disclosed herein may contain minor impurities that result, e.g., from the synthesis methods or the partial conversion of one crystalline form to another. Impurities may also be in the form of residual solvent or water that is present outside of the lattice.

In one embodiment, the Compound 1 oxalate salt or oxalic acid cocrystal has a purity of at least about 75%, at least about 80%, at least about 85%, at least about 90%, at least about 95%, at least about 97%, at least about 98%, or at least about 99%.

In one embodiment, the Compound 1 oxalate salt or oxalic acid cocrystal has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the Compound 1 oxalate salt or oxalic acid cocrystal has a purity of about 75% to about 99%. In one embodiment, the Compound 1 oxalate salt or oxalic acid cocrystal has a purity of about 80% to about 99%. In one embodiment, the Compound 1 oxalate salt or oxalic acid cocrystal has a purity of about 85% to about 99%. In one embodiment, the Compound 1 oxalate salt or oxalic acid cocrystal has a purity of about 90% to about 99%. In one embodiment, the Compound 1 oxalate salt or oxalic acid cocrystal has a purity of about 95% to about 99%. In one embodiment, the Compound 1 oxalate salt or oxalic acid cocrystal has a purity of about 97.5% to about 99%.

In one embodiment, the specific solid form of the Compound 1 oxalate salt or oxalic acid cocrystal with high purity is crystalline Form I. In some embodiments, the Compound 1 Form I is prepared according to the procedure described in Example 1.

### Compound 1 Form II

In some embodiments, the present disclosure provides a bromide salt or hydrobromide cocrystal of Compound 1. In some embodiments, the bromide salt of Compound 1 is crystalline. In some embodiments, the crystalline bromide salt is Compound 1 Form II bromide salt. In some embodiments, the cocrystal of Compound 1 is Compound 1 Form II hydrobromide cocrystal. In some embodiments, the crystalline form is a polymorph form. The bromide salt or hydrobromide cocrystal can have a ratio of Compound 1:hydrobromide of about 1:1, 1:2, or about 1:3, as exemplified by the structures below. In some embodiments, the ratio of Compound 1:hydrobromide in the salt or cocrystal is about 1:1.

In one embodiment, the present disclosure provides Compound 1 Form II. In some embodiments, the Compound 1 Form II exhibits an XRPD pattern comprising 1, 2, 3, 4, or 5 or more peaks at 3.74, 7.48, 8.84, 10.97, 11.23, 13.81, 15.00, 16.49, 17.05, 17.73, 18.78, 20.45, 21.46, 21.96, 24.52, 24.80, 25.66, 26.73, 28.85, 34.21, and 36.48 degrees two-theta with the margin of error of ±0.5; ±0.4; ±0.3; ±0.2; ±0.1; ±0.05; or less. In some embodiments, the Compound 1 Form II exhibits an XRPD pattern comprising peaks at 3.74, 7.48, 8.84, 10.97, 11.23, 13.81, 15.00, 16.49, 17.05, 17.73, 18.78, 20.45, 21.46, 21.96, 24.52, 24.80, 25.66, 26.73, 28.85, 34.21, and 36.48 degrees two-theta with the margin of error of 0.5; ±0.4; ±0.3; ±0.2; ±0.1; ±0.05; or less. In some embodiments, the Compound 1 Form II exhibits an XRPD pattern comprising peaks at 7.48, 10.97, 11.23, 15.00, 17.73, 18.78, 20.45, 21.46, 24.52, 24.80, and 25.66 degrees two-theta with the margin of error of ±0.5; ±0.4; ±0.3; ±0.2; ±0.1; ±0.05; or less. In some embodiments, the XRPD peaks have a margin of error of ±0.2. In some embodiments, the XRPD peaks have a margin of error of ±0.1. In yet another embodiment, the Compound 1 Form II exhibits an XRPD comprising peaks shown in Table 2 below.

TABLE 2
XRPD Table of Compound 1 Form II.

| | Relative |
|---|---|
| 2-Theta | Intensity % |
| 3.74 | 4.9 |
| 7.48 | 17.4 |
| 8.84 | 9.2 |
| 10.97 | 17.2 |
| 11.23 | 100.0 |
| 13.81 | 9.4 |
| 15.00 | 12.0 |
| 16.49 | 8.6 |
| 17.05 | 5.8 |
| 17.73 | 21.7 |
| 18.78 | 17.6 |
| 20.45 | 34.9 |
| 21.46 | 19.0 |
| 21.96 | 3.3 |
| 24.52 | 15.0 |
| 24.80 | 44.9 |
| 25.66 | 35.8 |
| 26.73 | 9.0 |
| 28.85 | 8.4 |
| 34.21 | 4.3 |
| 36.48 | 3.1 |

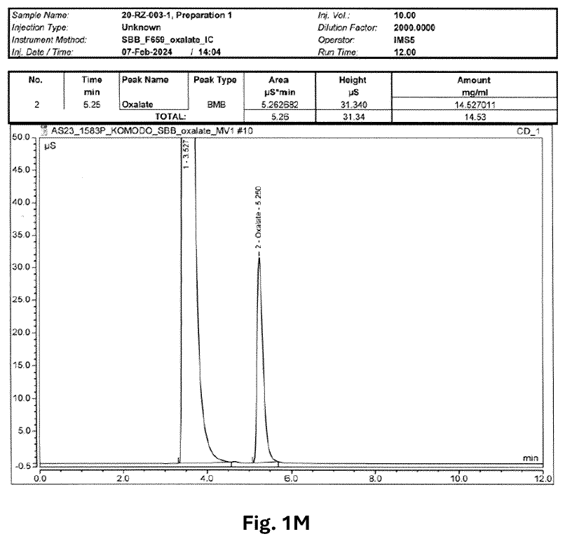

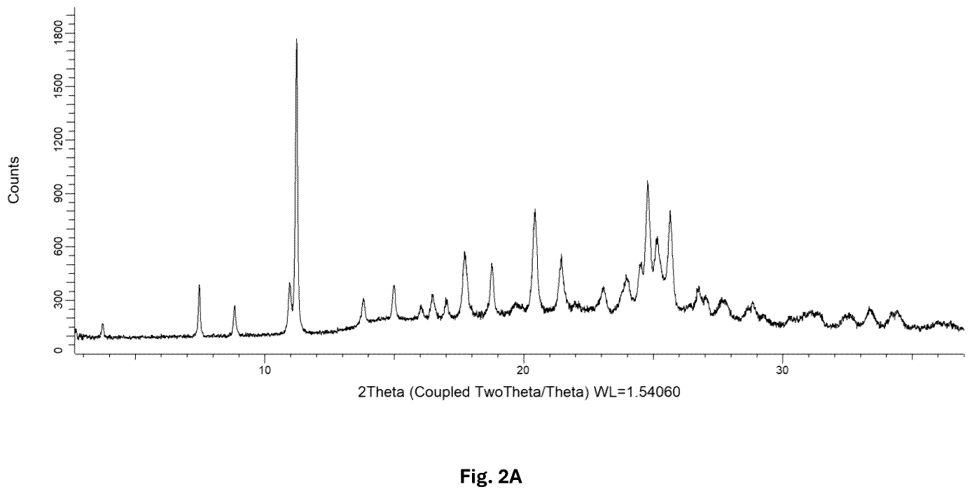

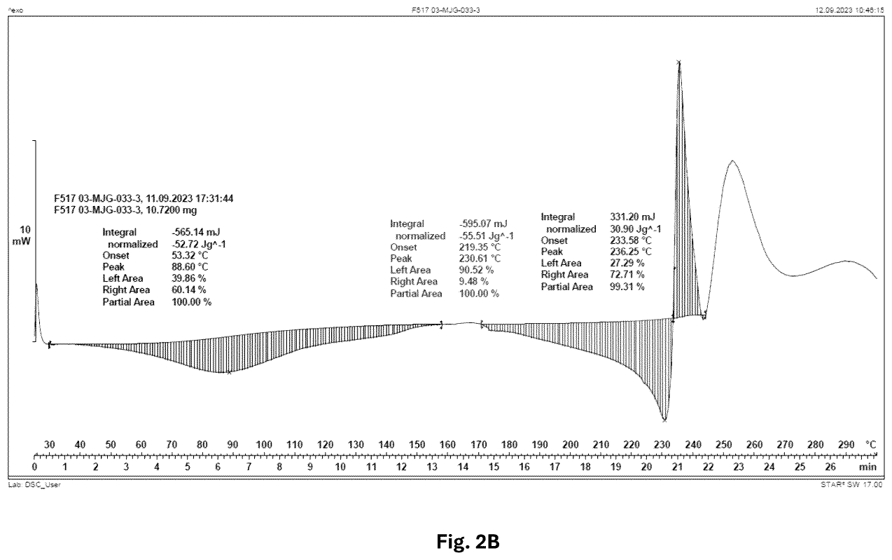

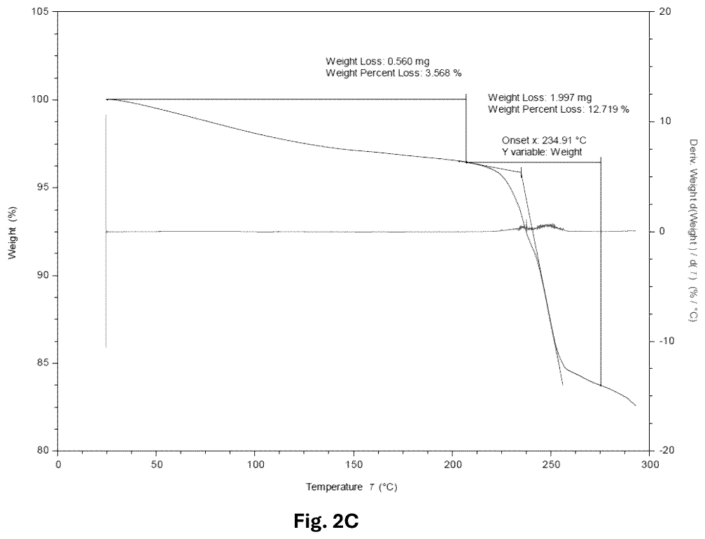

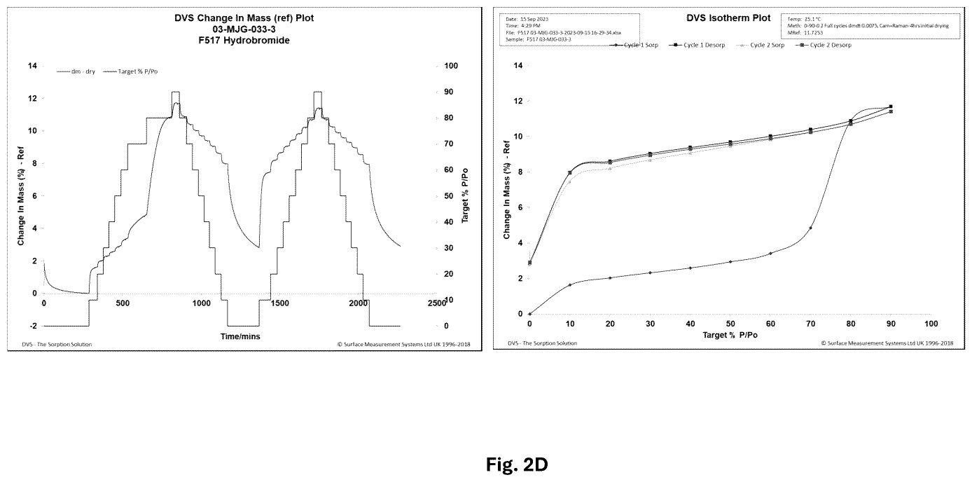

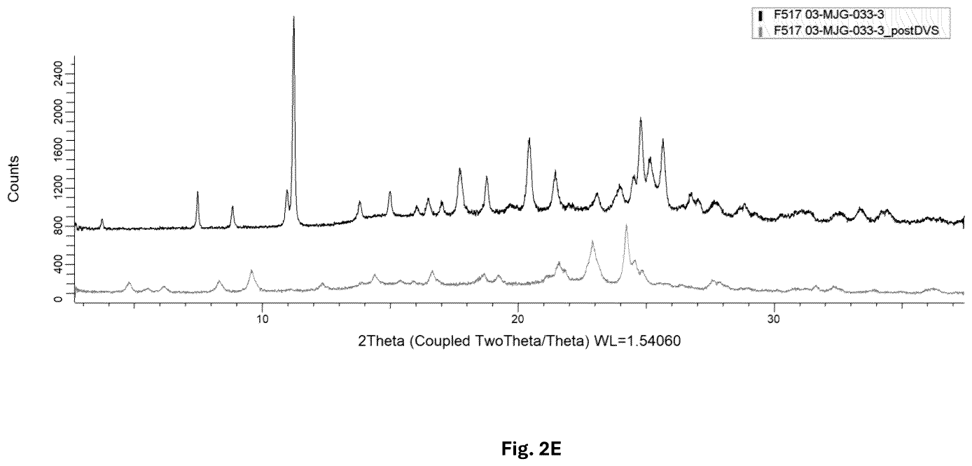

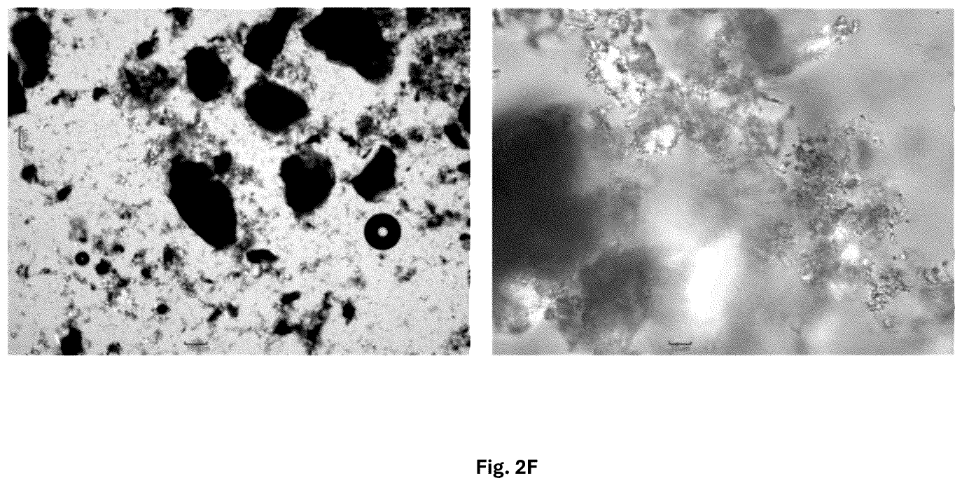

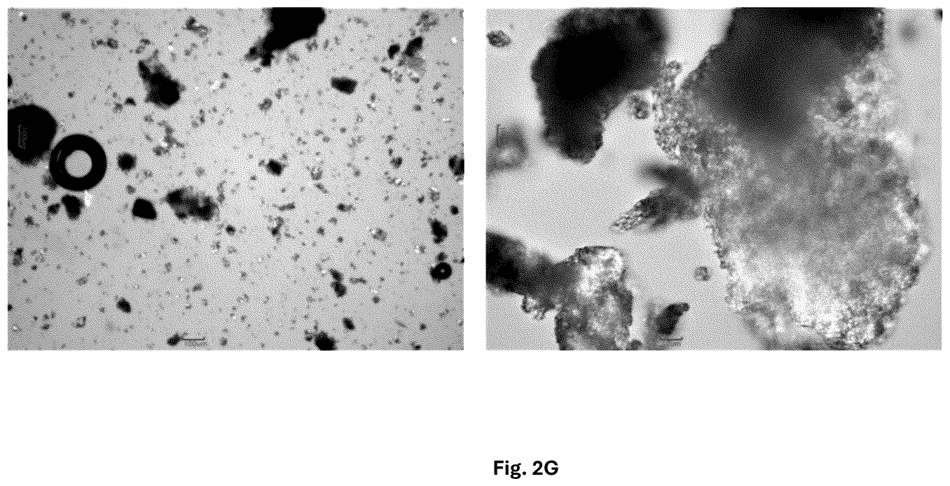

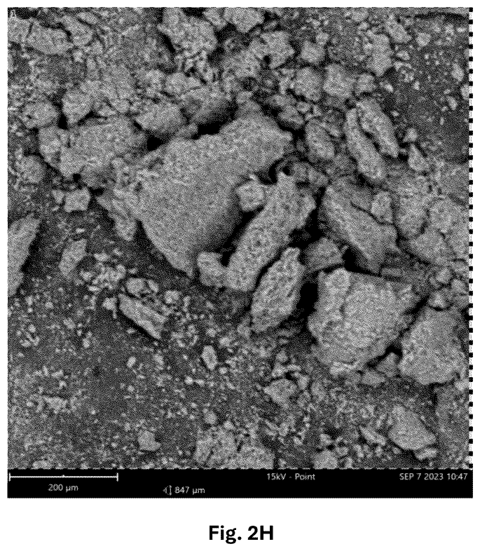

In some embodiments, the Compound 1 Form II exhibits an XRPD that is substantially similar to FIG. 2A.

In some embodiments, the Compound 1 Form II exhibits a DSC thermogram comprising a sharp endotherm that onsets at a temperature of 53° C., centered on about 89° C. with the margin of error of 10.0; ±5.0; ±4.0; ±3.0; ±2.0; ±1.0; or less. In some embodiments, the Compound 1 Form II exhibits a DSC thermogram comprising two additional overlapping endotherms with onset temperatures of about 219° C. and 234° C. with the margin of error of 10.0; ±5.0; ±4.0; ±3.0; ±2.0; ±1.0; or less. In some embodiments, the Compound 1 Form II exhibits a DSC thermogram that is substantially similar to FIG. 2B.

In some embodiments, the Compound 1 Form II exhibits a TGA thermogram that is substantially similar to FIG. 2C. In some embodiments, the TGA thermogram of the Compound 1 Form II exhibits a weight loss of about 1-10% from about 25° C. to 235° C. In some embodiments, the TGA thermogram of the Compound 1 Form II exhibits a weight loss of about 3.6% from about 25° C. to 235° C. In some embodiments, the TGA thermogram of the Compound 1 Form II exhibits a second weight loss of about 5-15% that onsets at a temperature of 235±5° C. In some embodiments, the TGA thermogram of the Compound 1 Form II exhibits a second weight loss of about 10-20% that onsets at a temperature of 235±5° C. In further embodiments, the TGA thermogram of the Compound 1 Form II exhibits a second weight loss of 12.7% that onsets at a temperature of about 235° C.

In some embodiments, the Compound 1 Form II exhibits a DVS isotherm plot that is substantially similar to FIG. 2D. In some embodiments, the Compound 1 Form II exhibits a mass increase of about 5-15% due to moisture intake at 80-90% relative humidity (RH). In some embodiments, the Compound 1 Form II exhibits a mass increase of about 10-15% due to moisture intake at 80-90% relative humidity (RH). In some embodiments, the Compound 1 Form II exhibits a mass increase of about 11.68% due to moisture intake at about 90% RH. In some embodiments, the Compound 1 Form II exhibits a mass increase of less that 5% due to moisture intake at 50-60% RH. In some embodiments, the Compound 1 Form II exhibits a mass increase of about 1-5% due to moisture intake at about 50% RH. In some embodiments, the Compound 1 Form II exhibits a mass increase of about 2.9% due to moisture intake at about 50% RH. In some embodiments, the Compound 1 Form II exhibits a mass loss on drying of less that 5% at about 0% RH. In some embodiments, the Compound 1 Form II exhibits a mass loss on drying of about 1-3% at about 0% RH. In some embodiments, the Compound 1 Form II exhibits a mass loss on drying of about 2% at about 0% RH.

In one embodiment, the Compound 1 bromide salt or hydrobromide cocrystal has a purity of at least about 75%, at least about 80%, at least about 85%, at least about 90%, at least about 95%, at least about 97%, at least about 98%, or at least about 99%.

In one embodiment, the Compound 1 bromide salt or hydrobromide cocrystal has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the Compound 1 bromide salt or hydrobromide cocrystal has a purity of about 75% to about 99%. In one embodiment, the Compound 1 bromide salt or hydrobromide cocrystal has a purity of about 80% to about 99%. In one embodiment, the Compound 1 bromide salt or hydrobromide cocrystal has a purity of about 85% to about 99%. In one embodiment, the Compound 1 bromide salt or hydrobromide cocrystal has a purity of about 90% to about 99%. In one embodiment, the Compound 1 bromide salt or hydrobromide cocrystal has a purity of about 95% to about 99%. In one embodiment, the Compound 1 bromide salt or hydrobromide cocrystal has a purity of about 97.5% to about 99%.

In one embodiment, the specific solid form of the Compound 1 bromide salt or hydrobromide cocrystal with high purity is crystalline Form II. In some embodiments, the Compound 1 Form II is prepared according to the procedure described in Example 2.

### Compound 1 Form III

In some embodiments, the present disclosure provides a chloride salt or hydrochloride cocrystal of Compound 1. In some embodiments, the chloride salt of Compound 1 is crystalline. In some embodiments, the crystalline chloride salt is Compound 1 Form III chloride salt. In some embodiments, the cocrystal of Compound 1 is Compound 1 Form III hydrochloride cocrystal. In some embodiments, the crystalline form is a polymorph form. The chloride salt or hydrochloride cocrystal can have a ratio of Compound 1:hydrochloride of about 1:1, 1:2, or about 1:3, as exemplified by the structures below. In some embodiments, the ratio of Compound 1:hydrochloride in the salt or cocrystal is about 1:1.

In one embodiment, the present disclosure provides Compound 1 Form III. In some embodiments, the Compound 1 Form III exhibits an XRPD pattern comprising 1, 2, 3, 4, or 5 or more peaks at 7.37, 8.97, 11.09, 13.89, 14.83, 16.28, 18.06, 20.44, 20.53, 24.84, 25.18, 25.22, and 26.14 degrees two-theta with the margin of error of 0.5; +0.4; +0.3; +0.2; +0.1; +0.05; or less. In some embodiments, the Compound 1 Form III exhibits an XRPD pattern comprising peaks at 7.37, 8.97, 11.09, 13.89, 14.83, 16.28, 18.06, 20.44, 20.53, 24.84, 25.18, 25.22, and 26.14 degrees two-theta with the margin of error of 0.5; +0.4; +0.3; +0.2; +0.1; +0.05; or less. In some embodiments, the Compound 1 Form III exhibits an XRPD pattern comprising 1, 2, 3, 4, or 5 or more peaks at 8.97, 11.09, 13.89, 14.83, 20.53, 25.18, 25.22, and 26.14 degrees two-theta with the margin of error of 0.5; +0.4; +0.3; +0.2; +0.1; +0.05; or less. In some embodiments, the XRPD peaks have a margin of error of +0.2. In some embodiments, the XRPD peaks have a margin of error of +0.1. In yet another embodiment, the Compound 1 Form III exhibits an XRPD comprising peaks shown in Table 3 below.

TABLE 3
XRPD Table of Compound 1 Form III.

| | Relative |
|---|---|
| 2-Theta | Intensity % |
| 7.37 | 11.4 |
| 8.97 | 37.3 |
| 11.09 | 100.0 |
| 13.89 | 35.2 |
| 14.83 | 20.1 |
| 16.28 | 13.0 |
| 18.06 | 14.1 |
| 20.44 | 17.3 |
| 20.53 | 22.5 |
| 24.84 | 15.2 |
| 25.18 | 39.7 |
| 25.22 | 38.1 |
| 26.14 | 19.7 |

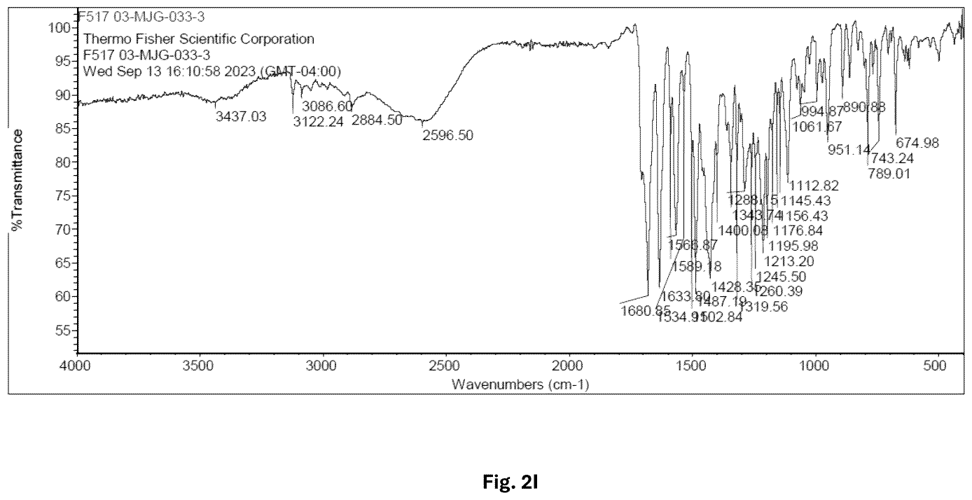

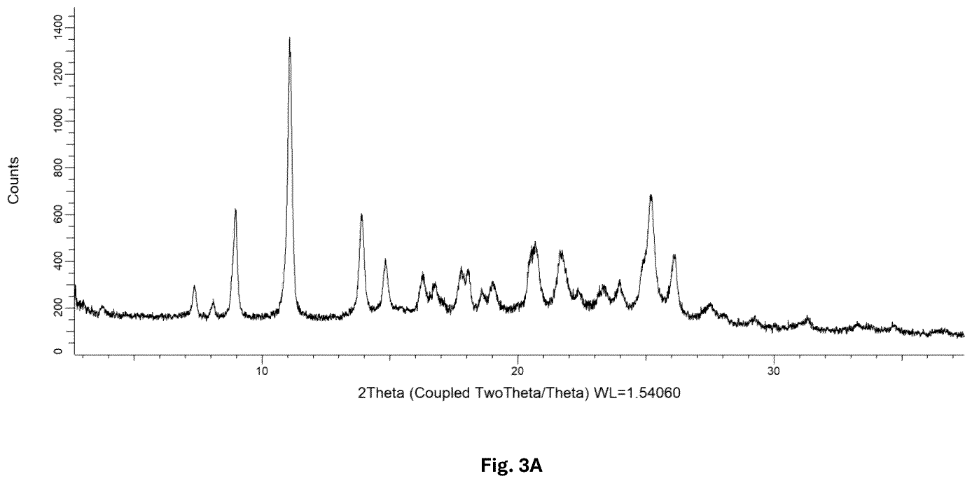

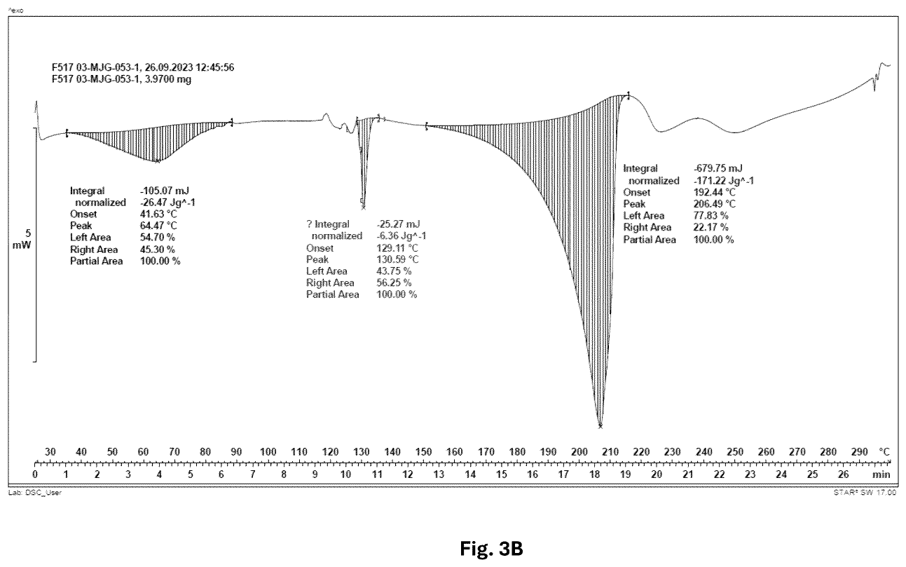

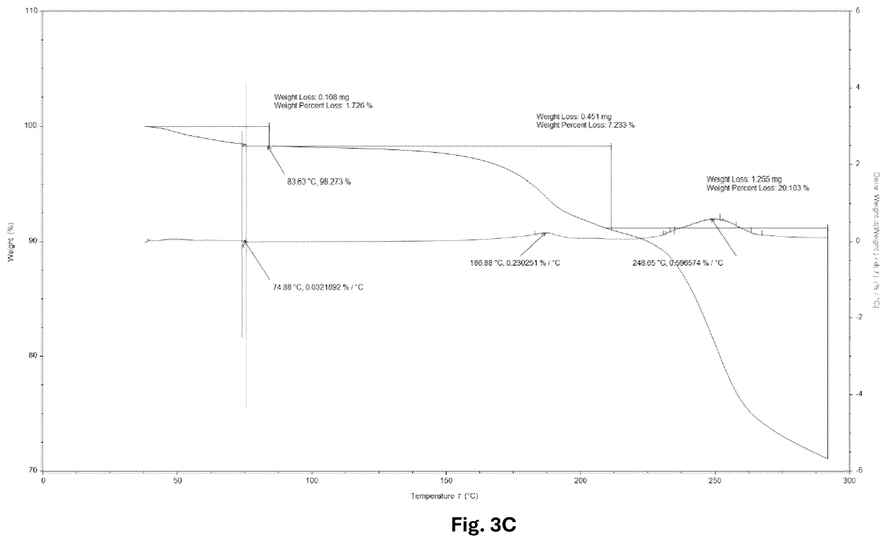

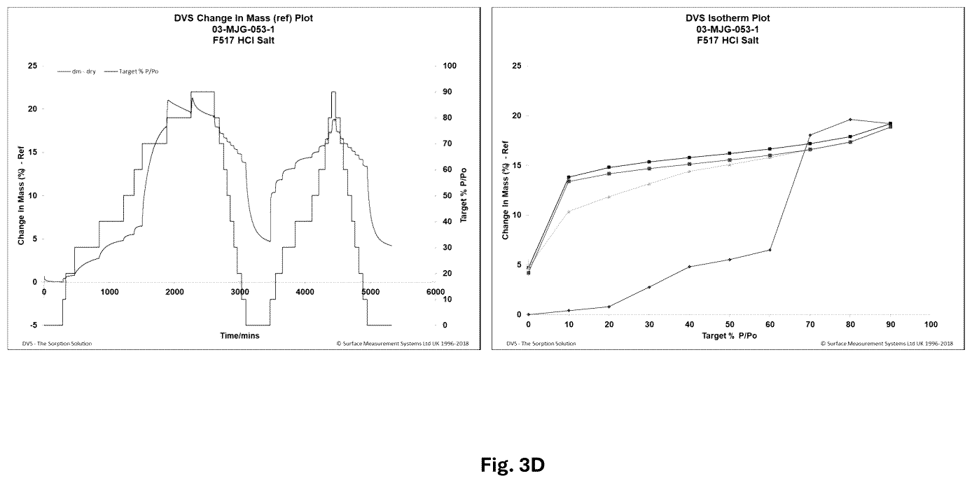

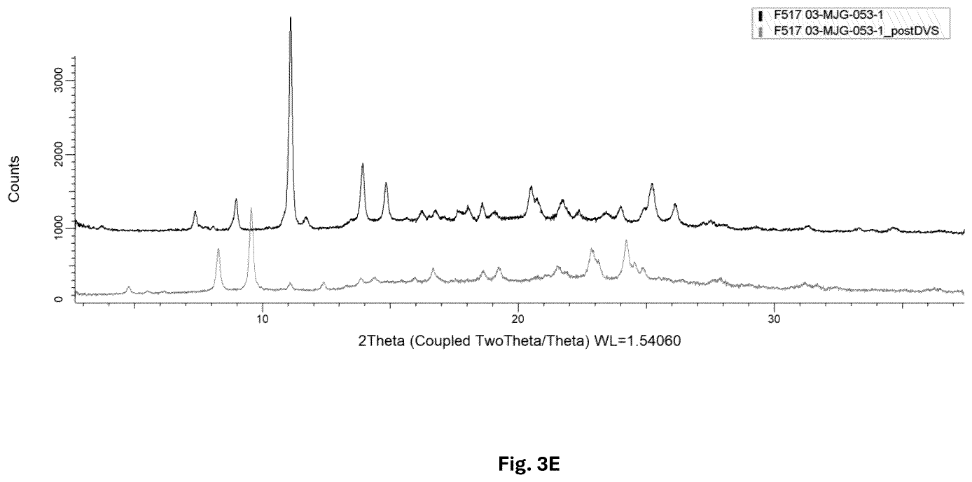

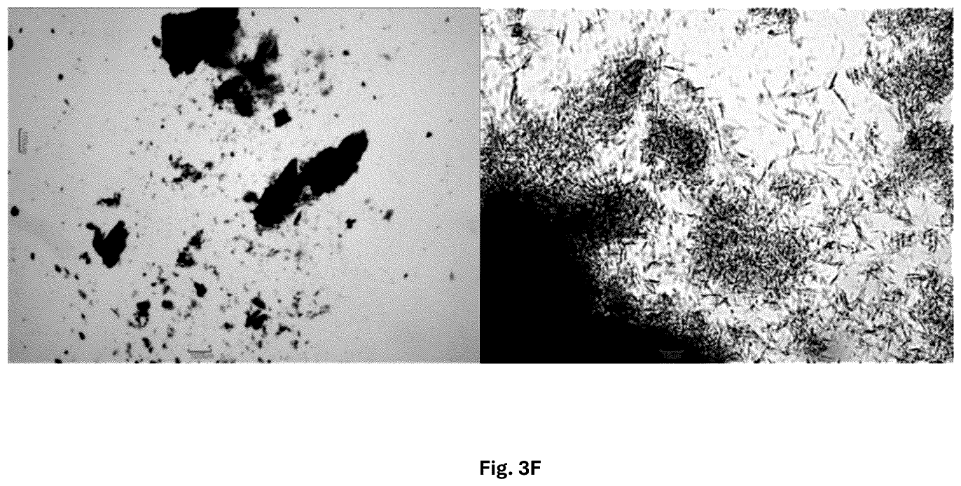

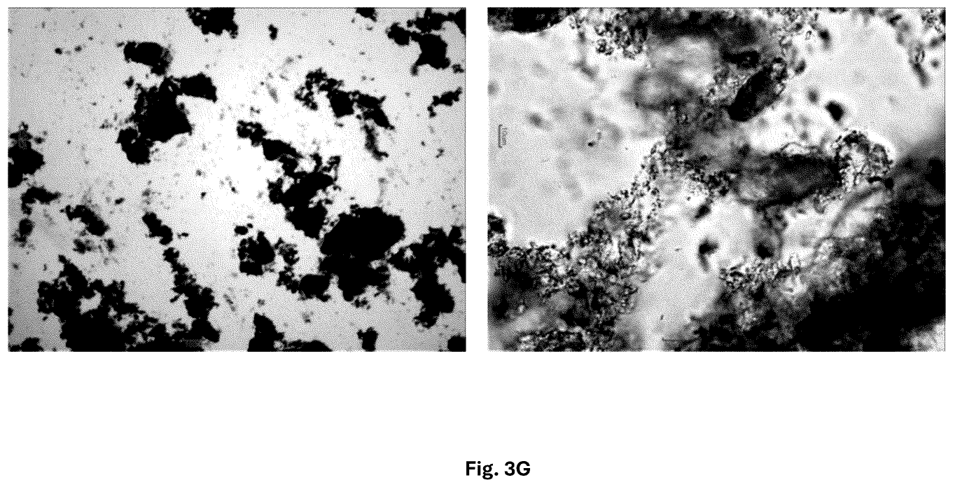

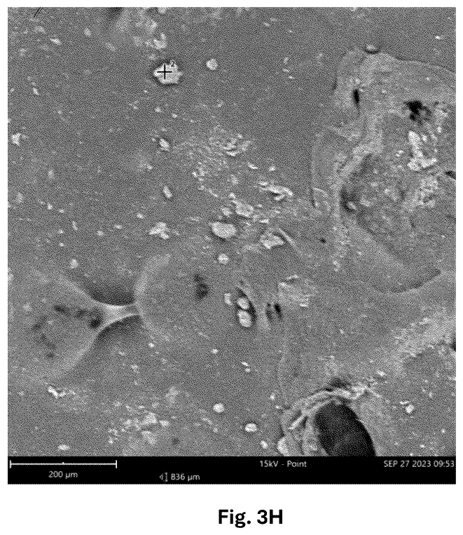

In some embodiments, the Compound 1 Form III exhibits an XRPD that is substantially similar to FIG. 3A.

In some embodiments, the Compound 1 Form III exhibits a DSC thermogram comprising an initial broad endotherm at 42° C. with the margin of error of +2.5; about +2.0; about +1.5; about +1.0; about +0.5; or less. In some embodiments, the Compound 1 Form III exhibits a DSC thermogram comprising an additional endotherm centered at 131° C. with the margin of error of +10.0; +5.0; +4.0; +3.0; +2.0; +1.0; or less. In further embodiments, the Compound 1 Form III exhibits a DSC thermogram comprising an additional endotherm that onsets at a temperature of 192° C. having a margin of error of 10.0; +5.0; +4.0; +3.0; +2.0; +1.0; or less. In some embodiments, the Compound 1 Form III exhibits a DSC thermogram that is substantially similar to FIG. 3B.

In some embodiments, the Compound 1 Form III exhibits a TGA thermogram that is substantially similar to FIG. 3C. In some embodiments, the TGA thermogram of the Compound 1 Form III exhibits a weight loss of about 1-5% from 25° C. to 180° C. In other embodiments, the TGA thermogram of the Compound 1 Form III exhibits a weight loss of about 1.7% from 25° C. to 179° C. In some embodiments, the TGA thermogram of the Compound 1 Form III exhibits additional weight loss events of about 5-10% centered on a temperature of 187±5° C. ° C. and about 15-25% centered on a temperature 249±5° C. In further embodiments, the TGA thermogram of the Compound 1 Form III exhibits additional weight loss events of about 7.2% centered on a temperature of about 187° C. and about 20.10% centered on a temperature of about 249° C.

In some embodiments, the Compound 1 Form III exhibits a DVS isotherm plot that is substantially similar to FIG. 3D. In some embodiments, the Compound 1 Form III exhibits a mass increase of about 15-25% due to moisture intake at 80-90% relative humidity (RH). In some embodiments, the Compound 1 Form III exhibits a mass increase of about 17-21% due to moisture intake at about 90% RH. In some embodiments, the Compound 1 Form III exhibits a mass increase of about 19.2% due to moisture intake at about 90% RH. In some embodiments, the Compound 1 Form III exhibits a mass increase of about 1-10% due to moisture intake at 50-60% RH. In some embodiments, the Compound 1 Form III exhibits a mass increase of about 3-7% due to moisture intake at about 50% RH. In some embodiments, the Compound 1 Form III exhibits a mass increase of about 5.5% due to moisture intake at about 50% RH. In some embodiments, the Compound 1 Form III exhibits a mass loss on drying of about 0.5-1.5% at about 0% RH. In some embodiments, the Compound 1 Form III exhibits a mass loss on drying of about 1% at about 0% RH.

In one embodiment, the Compound 1 chloride salt or hydrochloride cocrystal has a purity of at least about 75%, at least about 80%, at least about 85%, at least about 90%, at least about 95%, at least about 97%, at least about 98%, or at least about 99%.

In one embodiment, the Compound 1 chloride salt or hydrochloride cocrystal has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the Compound 1 chloride salt or hydrochloride cocrystal has a purity of about 75% to about 99%. In one embodiment, the Compound 1 chloride salt or hydrochloride cocrystal has a purity of about 80% to about 99%. In one embodiment, the Compound 1 chloride salt or hydrochloride cocrystal has a purity of about 85% to about 99%. In one embodiment, the Compound 1 chloride salt or hydrochloride cocrystal has a purity of about 90% to about 99%. In one embodiment, the Compound 1 chloride salt or hydrochloride cocrystal has a purity of about 95% to about 99%. In one embodiment, the Compound 1 chloride salt or hydrochloride cocrystal has a purity of about 97.5% to about 99%.

In one embodiment, the specific solid form of the Compound 1 chloride salt or hydrochloride cocrystal with high purity is crystalline Form III. In some embodiments, the Compound 1 Form III is prepared according to the procedure described in Example 3.

### Compound 1 Form IV

In some embodiments, the present disclosure provides an phosphate salt or phosphoric acid cocrystal of Compound 1. In some embodiments, the phosphate salt of Compound 1 is crystalline. In some embodiments, the crystalline phosphate salt is Compound 1 Form IV phosphate salt. This disclosure encompasses all phosphate anions. In some embodiments, the cocrystal of Compound 1 is Compound 1 Form IV phosphoric acid cocrystal. In some embodiments, the crystalline form is a polymorph form. The phosphate salt or phosphoric acid cocrystal can have a ratio of Compound 1:phosphoric acid of about 1:1, 1:2, or about 1:3, as exemplified by the structures below. In some embodiments, the ratio of Compound 1:phosphoric acid in the salt or cocrystal is about 1:1.

In one embodiment, the present disclosure provides Compound 1 Form IV. In some embodiments, the Compound 1 Form IV exhibits an XRPD pattern comprising 1, 2, 3, 4, or 5 or more peaks at 3.66, 6.26, 7.34, 11.02, 12.56, 14.15, 14.39, 14.99, 15.32, 16.19, 17.72, 18.38, 18.86, 19.50, 19.77, 20.76, 21.08, 21.29, 22.08, 22.33, 22.76, 22.83, 23.14, 23.39, 23.68, 24.14, 24.52, 26.64, and 28.76 degrees two-theta with the margin of error of 0.5; ±0.4; ±0.3; ±0.2; ±0.1; ±0.05; or less. In some embodiments, the Compound 1 Form IV exhibits an XRPD pattern comprising peaks at 3.66, 6.26, 7.34, 11.02, 12.56, 14.15, 14.39, 14.99, 15.32, 16.19, 17.72, 18.38, 18.86, 19.50, 19.77, 20.76, 21.08, 21.29, 22.08, 22.33, 22.76, 22.83, 23.14, 23.39, 23.68, 24.14, 24.52, 26.64, and 28.76 degrees two-theta with the margin of error of 0.5; ±0.4; ±0.3; ±0.2; ±0.1; ±0.05; or less. In some embodiments, the Compound 1 Form IV exhibits an XRPD pattern comprising peaks at 7.34, 11.02, 17.72, 18.38, 18.86, 19.50, 19.77, 20.76, 21.29, 22.08, 22.76, 22.83, 23.14, 23.39, 23.68, 24.14, and 24.52 degrees two-theta with the margin of error of 0.5; ±0.4; ±0.3; ±0.2; ±0.1; ±0.05; or less. In some embodiments, the XRPD peaks have a margin of error of ±0.2. In some embodiments, the XRPD peaks have a margin of error of ±0.1. In yet another embodiment, the Compound 1 Form IV exhibits an XRPD comprising peaks shown in Table 4 below.

TABLE 4
XRPD Table of Compound 1 Form IV.

| | Relative |
|---|---|
| 2-Theta | Intensity % |
| 3.66 | 13.7 |
| 6.26 | 13.9 |
| 7.34 | 45.4 |
| 11.02 | 100.0 |
| 12.56 | 12.3 |
| 14.15 | 12.3 |
| 14.39 | 11.0 |
| 14.99 | 16.1 |
| 15.32 | 13.5 |
| 16.19 | 10.5 |
| 17.72 | 35.8 |
| 18.38 | 49.9 |
| 18.86 | 24.4 |
| 19.50 | 23.0 |
| 19.77 | 38.5 |
| 20.76 | 22.4 |
| 21.08 | 17.8 |
| 21.29 | 23.0 |
| 22.08 | 20.7 |
| 22.33 | 14.9 |
| 22.76 | 31.8 |
| 22.83 | 47.1 |
| 23.14 | 41.1 |
| 23.39 | 28.7 |
| 23.68 | 21.8 |
| 24.14 | 32.1 |
| 24.52 | 36.9 |
| 26.64 | 15.8 |
| 28.76 | 13.1 |

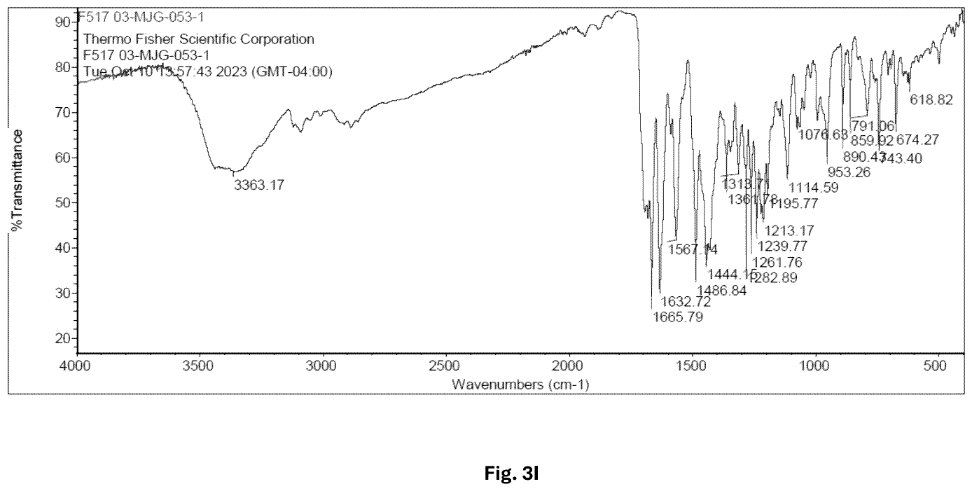

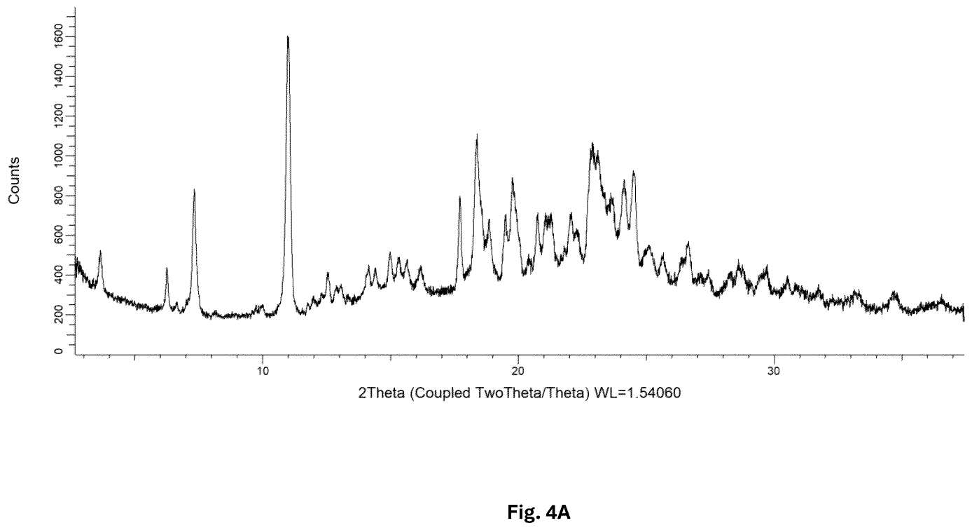

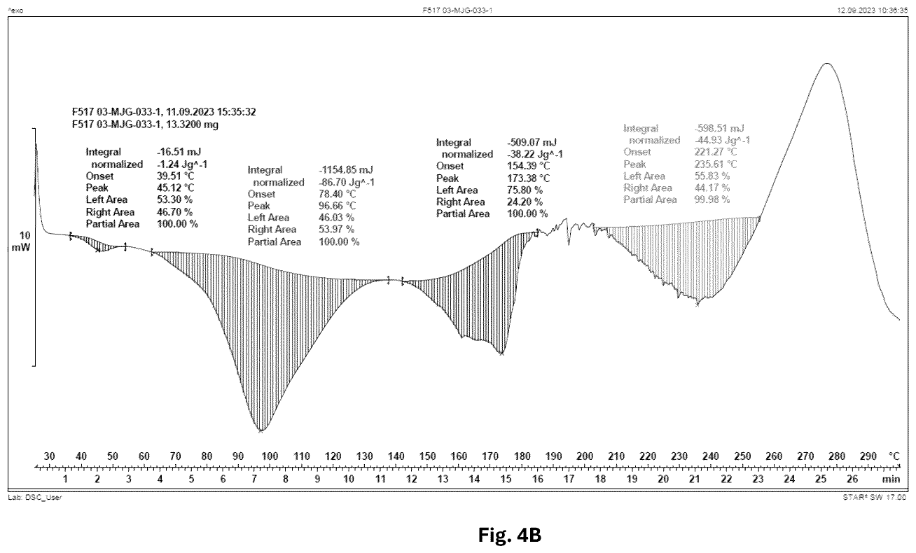

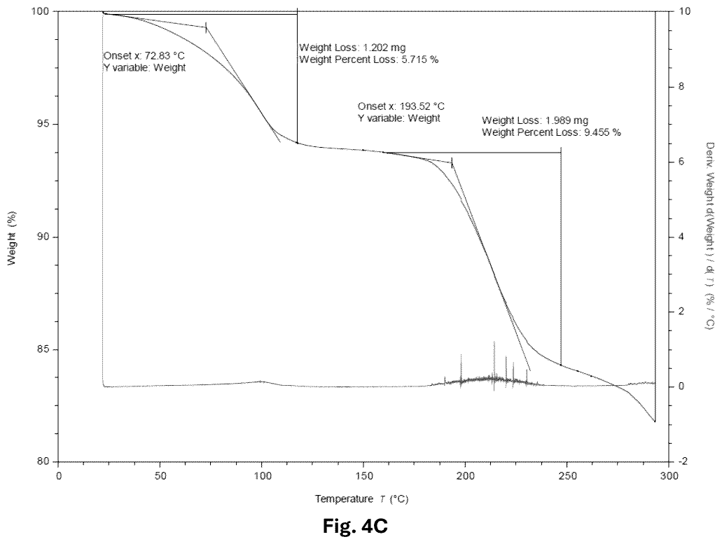

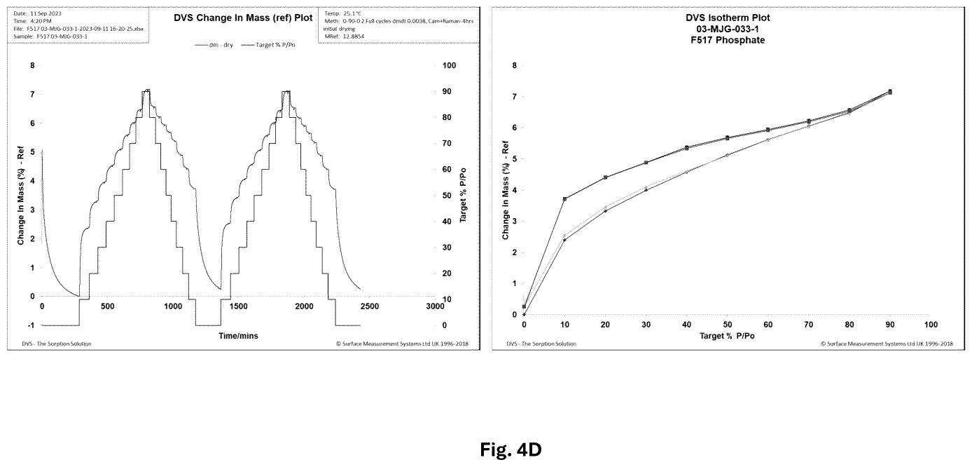

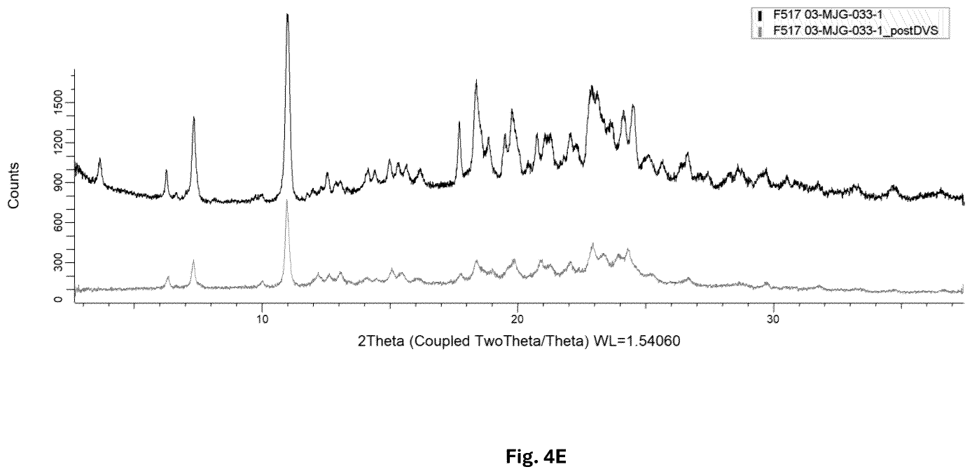

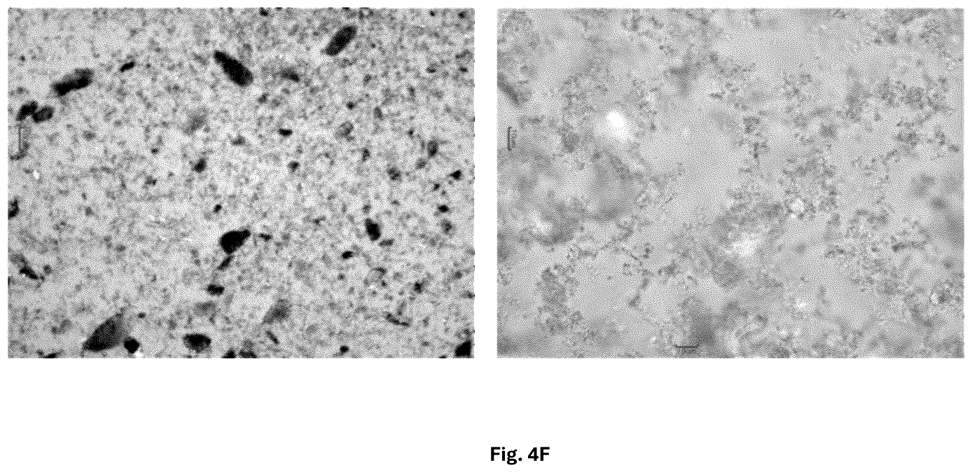

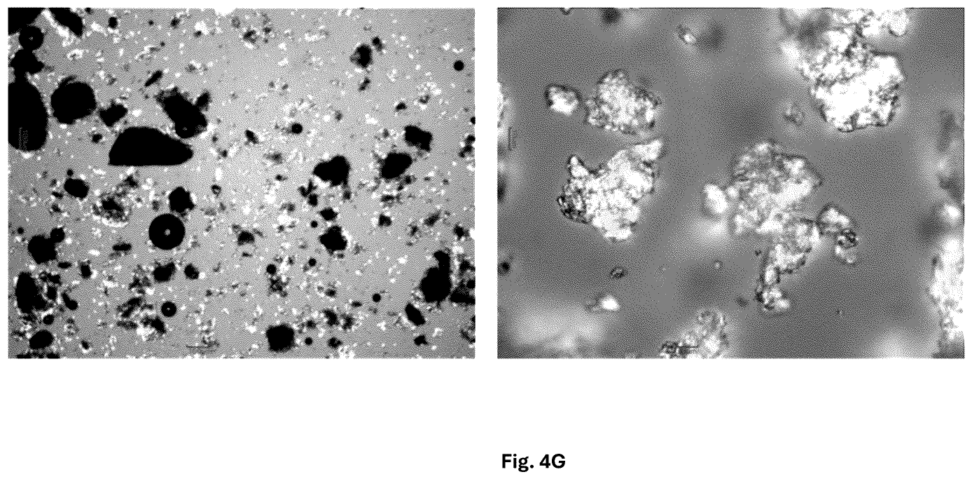

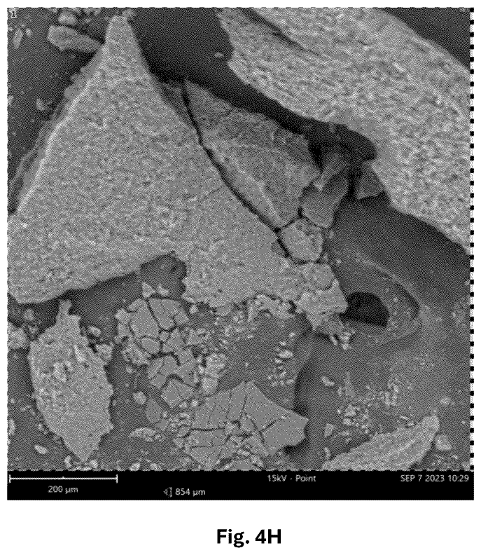

In some embodiments, the Compound 1 Form IV exhibits an XRPD that is substantially similar to FIG. 4A.

In some embodiments, the Compound 1 Form IV exhibits a DSC thermogram comprising a first small endotherm that onsets at a temperature of 40° C. and a second broad endotherm that onsets at a temperature of 78° C. having a margin of error of 10.0; ±5.0; ±4.0; ±3.0; ±2.0; ±1.0; or less. In some embodiments, the Compound 1 Form IV exhibits a DSC thermogram comprising an additional endotherm that onsets at a temperature of 154° C. with the margin of error of 10.0; ±5.0; ±4.0; ±3.0; ±2.0; ±1.0; or less. In further embodiments, the Compound 1 Form IV exhibits a DSC thermogram comprising an additional endotherm that onsets at a temperature of 221° C. having a margin of error of ±10.0; ±5.0; ±4.0; ±3.0; ±2.0; ±1.0; or less. In some embodiments, the Compound 1 Form IV exhibits a DSC thermogram that is substantially similar to FIG. 4B.

In some embodiments, the Compound 1 Form IV exhibits a TGA thermogram that is substantially similar to FIG. 4C. In other embodiments, the TGA thermogram of the Compound 1 Form IV exhibits a weight loss of about 1-10% that onsets at a temperature of 73±5° C. In other embodiments, the TGA thermogram of the Compound 1 Form IV exhibits a weight loss of about 5.72% that onsets at a temperature of 73° C. In some embodiments, the TGA thermogram of the Compound 1 Form IV exhibits an additional weight loss of about 5-15% that onsets at a temperature of 194±5° C. In further embodiments, the TGA thermogram of the Compound 1 Form IV exhibits an additional weight loss of 9.46% that onsets at a temperature of about 194° C.

In some embodiments, the Compound 1 Form IV exhibits a DVS isotherm plot that is substantially similar to FIG. 4D. In some embodiments, the Compound 1 Form IV exhibits a mass increase of about 5-15% due to moisture intake at 80-90% relative humidity (RH). In some embodiments, the Compound 1 Form IV exhibits a mass increase of about 5-10% due to moisture intake at about 90% RH. In some embodiments, the Compound 1 Form IV exhibits a mass increase of about 7.2% due to moisture intake at about 90% RH. In some embodiments, the Compound 1 Form IV exhibits a mass increase of about 2-10% due to moisture intake at 50-60% RH. In some embodiments, the Compound 1 Form IV exhibits a mass increase of about 3-7% due to moisture intake at about 50% RH. In some embodiments, the Compound 1 Form IV exhibits a mass increase of about 5.1% due to moisture intake at about 50% RH. In some embodiments, the Compound 1 Form IV exhibits a mass loss on drying of about 10% or less at about 0% RH. In some embodiments, the Compound 1 Form IV exhibits a mass loss on drying of about 3-7% at about 0% RH. In some embodiments, the Compound 1 Form IV exhibits a mass loss on drying of about 5% at about 0% RH.

In one embodiment, the Compound 1 phosphate salt or phosphoric acid cocrystal has a purity of at least about 75%, at least about 80%, at least about 85%, at least about 90%, at least about 95%, at least about 97%, at least about 98%, or at least about 99%.

In one embodiment, the Compound 1 phosphate salt or phosphoric acid cocrystal has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the Compound 1 phosphate salt or phosphoric acid cocrystal has a purity of about 75% to about 99%. In one embodiment, the Compound 1 phosphate salt or phosphoric acid cocrystal has a purity of about 80% to about 99%. In one embodiment, the Compound 1 phosphate salt or phosphoric acid cocrystal has a purity of about 85% to about 99%. In one embodiment, the Compound 1 phosphate salt or phosphoric acid cocrystal has a purity of about 90% to about 99%. In one embodiment, the Compound 1 phosphate salt or phosphoric acid cocrystal has a purity of about 95% to about 99%. In one embodiment, the Compound 1 phosphate salt or phosphoric acid cocrystal has a purity of about 97.5% to about 99%.

In one embodiment, the specific solid form of the Compound 1 phosphate salt or phosphoric acid cocrystal with high purity is crystalline Form I. In some embodiments, the Compound 1 Form I is prepared according to the procedure described in Example 4.

### Compound 1 Freebase Form A

In some embodiments, the present disclosure provides a freebase of Compound 1 (“Compound 1 Freebase”). In some embodiments, the present disclosure provides a crystalline form of Compound 1 Freebase. In some embodiments, the crystalline freebase form is a Form A freebase.

In one embodiment, the present disclosure provides Compound 1 Form A freebase. In some embodiments, the Compound 1 Form A freebase exhibits an XRPD pattern comprising 1, 2, 3, 4, or 5 or more peaks at 6.45, 8.71, 11.02, 11.54, 12.11, 12.94, 14.63, 15.73, 16.80, 17.84, 18.44, 19.36, 19.77, 20.38, 20.54, 20.81, 21.10, 22.27, 27.70, 22.97, 24.48, 25.63, 25.92, 27.42, 27.91, 29.54, and 30.12 degrees two-theta with the margin of error of 0.5; ±0.4; ±0.3; ±0.2; ±0.1; ±0.05; or less. In some embodiments, the Compound 1 Form A freebase exhibits an XRPD pattern comprising peaks at 6.45, 8.71, 11.02, 11.54, 12.11, 12.94, 14.63, 15.73, 16.80, 17.84, 18.44, 19.36, 19.77, 20.38, 20.54, 20.81, 21.10, 22.27, 27.70, 22.97, 24.48, 25.63, 25.92, 27.42, 27.91, 29.54, and 30.12 degrees two-theta with the margin of error of ±0.5; ±0.4; ±0.3; ±0.2; ±0.1; ±0.05; or less. In some embodiments, the Compound 1 Form A freebase exhibits an XRPD pattern comprising peaks at 6.45, 8.71, 11.02, 11.54, 12.11, 12.94, 14.63, 17.84, 18.44, 19.36, 19.77, 20.38, 20.54, 20.81, 21.10, 22.27, 27.70, 22.97, 24.48, 25.63, and 27.42 degrees two-theta with the margin of error of ±0.5; ±0.4; ±0.3; ±0.2; ±0.1; ±0.05; or less. In yet another embodiment, the Compound 1 Form A freebase salt exhibits an XRPD comprising peaks shown in Table 5 below.

TABLE 5
XRPD Table of Compound 1 Form A freebase.

| | Relative |
|---|---|
| 2-Theta | Intensity % |
| 6.45 | 26.6 |
| 8.71 | 65.7 |
| 11.02 | 100.0 |
| 11.54 | 43.7 |
| 12.11 | 21.2 |
| 12.94 | 47.3 |
| 14.63 | 49.4 |
| 15.73 | 17.5 |
| 16.80 | 13.1 |
| 17.84 | 25.1 |
| 18.44 | 32.9 |
| 19.36 | 35.4 |
| 19.77 | 39.1 |
| 20.38 | 88.6 |
| 20.54 | 62.6 |
| 20.81 | 24.9 |
| 21.10 | 29.8 |
| 22.27 | 39.3 |
| 22.70 | 26.7 |
| 22.97 | 86.8 |
| 24.48 | 34.3 |
| 25.63 | 45.8 |
| 25.92 | 17.4 |
| 27.42 | 19.8 |
| 27.91 | 15.8 |
| 29.54 | 17.4 |
| 30.12 | 12.4 |

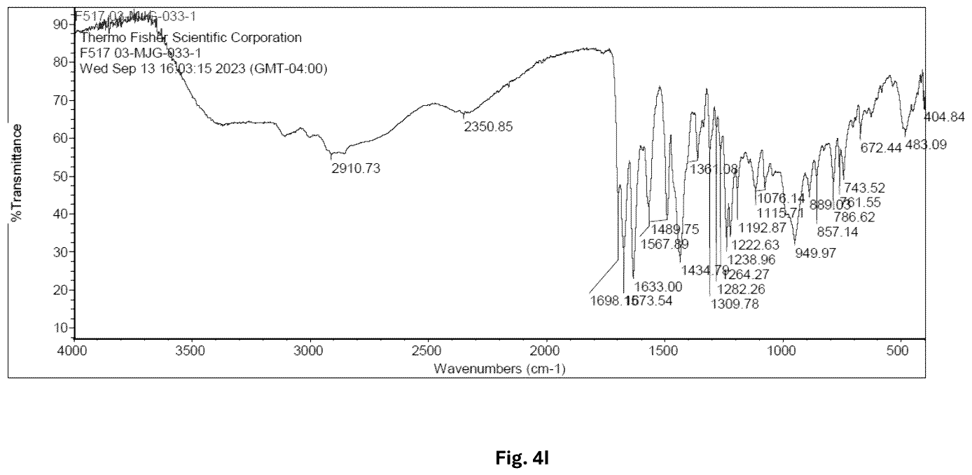

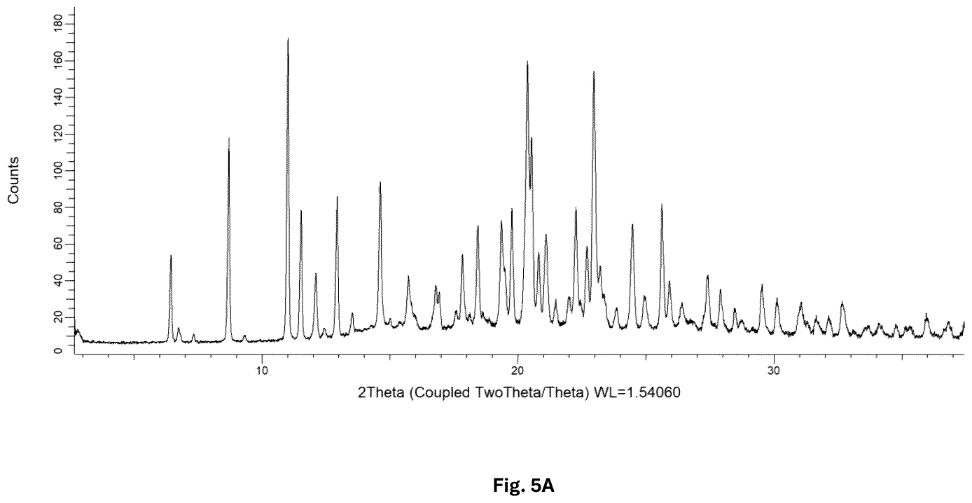

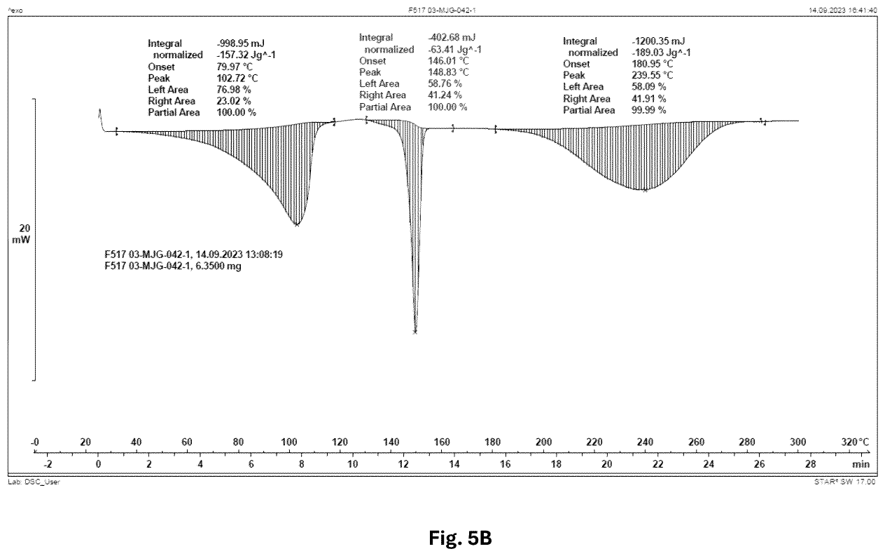

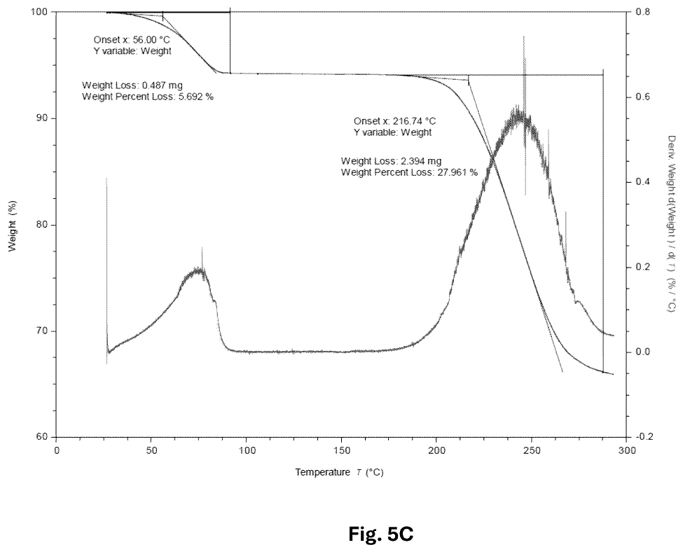

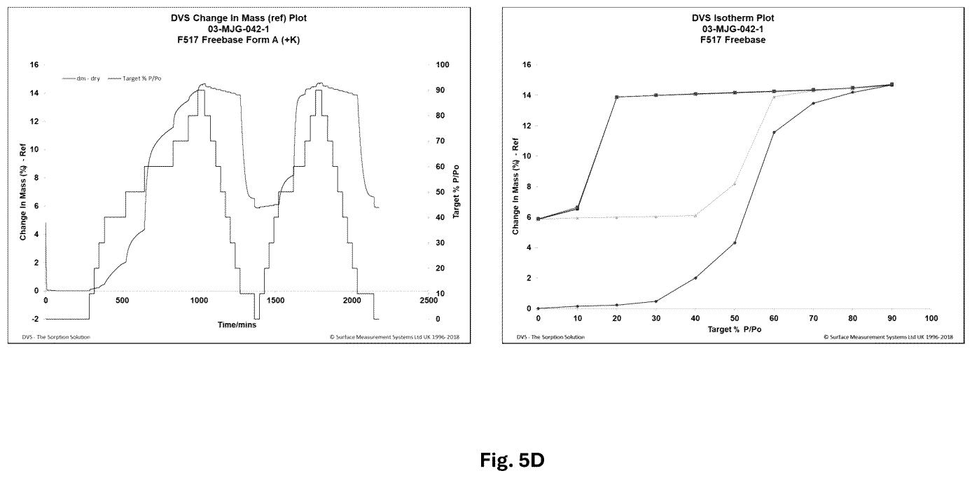

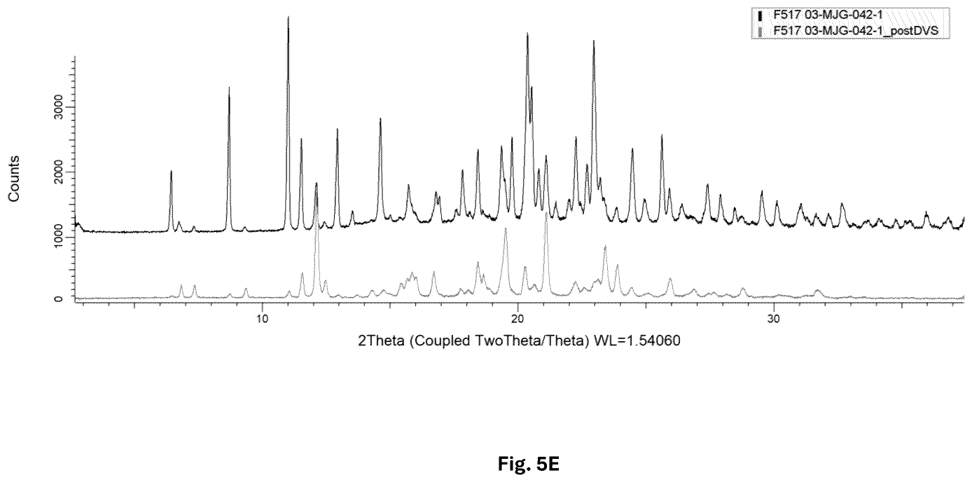

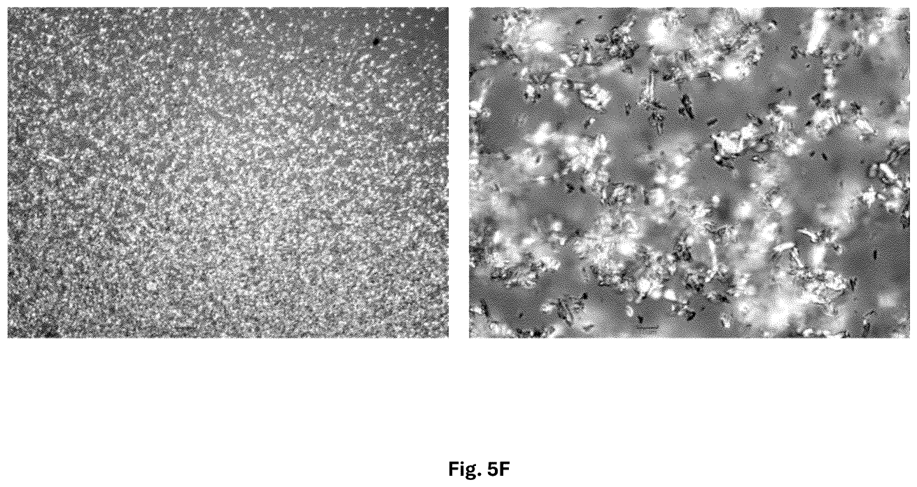

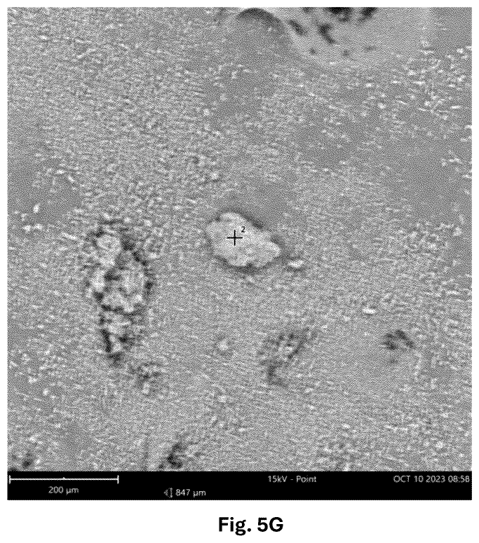

In some embodiments, the Compound 1 Form A freebase exhibits an XRPD that is substantially similar to FIG. 5A.

In some embodiments, the Compound 1 Form A freebase exhibits a DSC thermogram comprising a broad endotherm that onsets at a temperature of 80° C. and a second broad endotherm that onsets at a temperature of 78° C. having a margin of error of 10.0; ±5.0; ±4.0; ±3.0; ±2.0; ±1.0; or less. In some embodiments, the Compound 1 Form A freebase exhibits a DSC thermogram comprising an additional endotherm that onsets at a temperature of 146° C. with the margin of error of ±10.0; ±5.0; ±4.0; ±3.0; ±2.0; ±1.0; or less. In further embodiments, the Compound 1 Form A freebase exhibits a DSC thermogram comprising an additional endotherm that onsets at a temperature of 181° C. having a margin of error of 10.0; ±5.0; ±4.0; ±3.0; ±2.0; ±1.0; or less. In some embodiments, the Compound 1 Form A freebase exhibits a DSC thermogram that is substantially similar to FIG. 5B.

In some embodiments, the Compound 1 Form A freebase exhibits a TGA thermogram that is substantially similar to FIG. 5C. In some embodiments, the TGA thermogram of the Compound 1 Form A freebase exhibits a weight loss of about 1-10% that onsets at a temperature of 56±5° C. In some embodiments, the TGA thermogram of the Compound 1 Form A freebase exhibits a weight loss of about 3-7% that onsets at a temperature of 56±5° C. In other embodiments, the TGA thermogram of the Compound 1 Form A freebase exhibits a weight loss of about 5.69% that onsets at a temperature of 56° C. In some embodiments, the TGA thermogram of the Compound 1 Form A freebase exhibits an additional weight loss of 20-30% that onsets at a temperature of 217±5° C. In further embodiments, the TGA thermogram of the Compound 1 Form A freebase exhibits an additional weight loss of 27.96% that onsets at a temperature of about 217° C.

In some embodiments, the Compound 1 Form A freebase exhibits a DVS isotherm plot that is substantially similar to FIG. 5D. In some embodiments, the Compound 1 Form A freebase exhibits a mass increase of about 10-20% due to moisture intake at 80-90% relative humidity (RH). In some embodiments, the Compound 1 Form A freebase exhibits a mass increase of about 14.7% due to moisture intake at about 90% RH. In some embodiments, the Compound 1 Form A freebase exhibits a mass increase of about 1-10% due to moisture intake at 50-60% RH. In some embodiments, the Compound 1 Form A freebase exhibits a mass increase of about 3-7% due to moisture intake at about 50% RH. In some embodiments, the Compound 1 Form A freebase exhibits a mass increase of about 4.3% due to moisture intake at about 50% RH. In some embodiments, the Compound 1 Form A freebase exhibits a mass loss on drying of less than 10% at about 0% RH. In some embodiments, the Compound 1 Form A freebase exhibits a mass loss on drying of about 3-7% at about 0% RH. In some embodiments, the Compound 1 Form A freebase exhibits a mass loss on drying of about 5% at about 0% RH.

In one embodiment, the Compound 1 Form A freebase has a purity of at least about 75%, at least about 80%, at least about 85%, at least about 90%, at least about 95%, at least about 97%, at least about 98%, or at least about 99%.

In one embodiment, the Compound 1 Form A freebase has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the Compound 1 Form A freebase has a purity of about 75% to about 99%. In one embodiment, the Compound 1 Form A freebase has a purity of about 80% to about 99%. In one embodiment, the Compound 1 Form A freebase has a purity of about 85% to about 99%. In one embodiment, the Compound 1 Form A freebase has a purity of about 90% to about 99%. In one embodiment, the Compound 1 Form A freebase has a purity of about 95% to about 99%. In one embodiment, the Compound 1 Form A freebase has a purity of about 97.5% to about 99%.

In one embodiment, the specific solid form of the Compound 1 Form A freebase with high purity is crystalline Form A freebase. In some embodiments, the Compound 1 Form A freebase is prepared according to the procedure described in Example 5.

### Compound 1 Freebase Form C

In some embodiments, the present disclosure provides a freebase of Compound 1 (“Compound 1 Freebase”). In some embodiments, the present disclosure provides a crystalline form of Compound 1 Freebase. In some embodiments, the crystalline freebase form is a Form C freebase.

In one embodiment, the present disclosure provides Compound 1 Form C freebase. In some embodiments, the Compound 1 Form C freebase exhibits an XRPD pattern comprising 1, 2, 3, 4, or 5 or more peaks at 5.48, 11.20, 12.55, 13.06, 18.05, 19.41, 21.48, 21.58, 21.87, 22.75, 23.65, 23.99, 24.79, 28.05, and 28.13 degrees two-theta with the margin of error of 0.5; ±0.4; ±0.3; ±0.2; ±0.1; ±0.05; or less. In some embodiments, the Compound 1 Form C freebase exhibits an XRPD pattern comprising peaks at 5.48, 11.20, 12.55, 13.06, 18.05, 19.41, 21.48, 21.58, 21.87, 22.75, 23.65, 23.99, 24.79, 28.05, and 28.13 degrees two-theta with the margin of error of 0.5; ±0.4; ±0.3; ±0.2; ±0.1; ±0.05; or less. In some embodiments, the Compound 1 Form C freebase exhibits an XRPD pattern comprising peaks at 5.48, 11.20, 12.55, 18.05, 19.41, 21.48, 21.87, 22.75, and 28.05 degrees two-theta with the margin of error of 0.5; ±0.4; ±0.3; ±0.2; ±0.1; ±0.05; or less. In some embodiments, the XRPD peaks have a margin of error of 0.2. In some embodiments, the XRPD peaks have a margin of error of 0.1. In yet another embodiment, the Compound 1 Form C freebase exhibits an XRPD comprising peaks shown in Table 6 below.

TABLE 6
XRPD Table of Compound 1 Form C freebase.

| | Relative |
|---|---|
| 2-Theta | Intensity % |
| 5.48 | 95.6 |
| 11.20 | 23.9 |
| 12.55 | 69.2 |
| 13.06 | 14.2 |
| 18.05 | 63.0 |
| 19.41 | 76.9 |
| 21.48 | 26.2 |
| 21.58 | 14.4 |
| 21.87 | 100.0 |
| 22.75 | 14.6 |
| 23.65 | 11.2 |
| 23.99 | 11.5 |
| 24.79 | 10.9 |
| 28.05 | 18.2 |
| 28.13 | 13.2 |

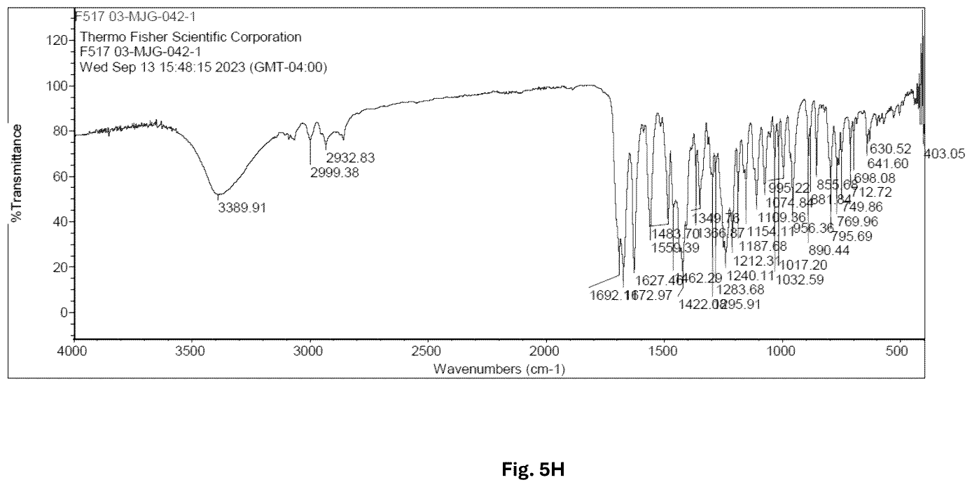

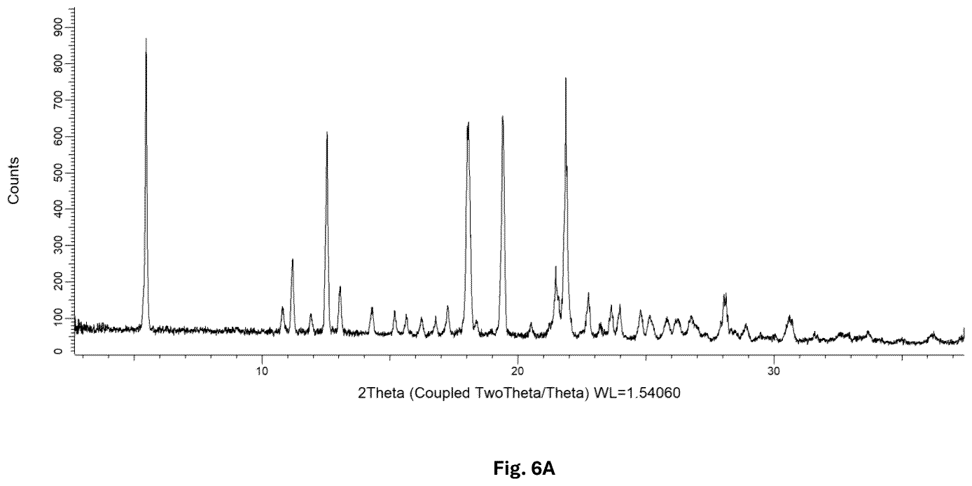

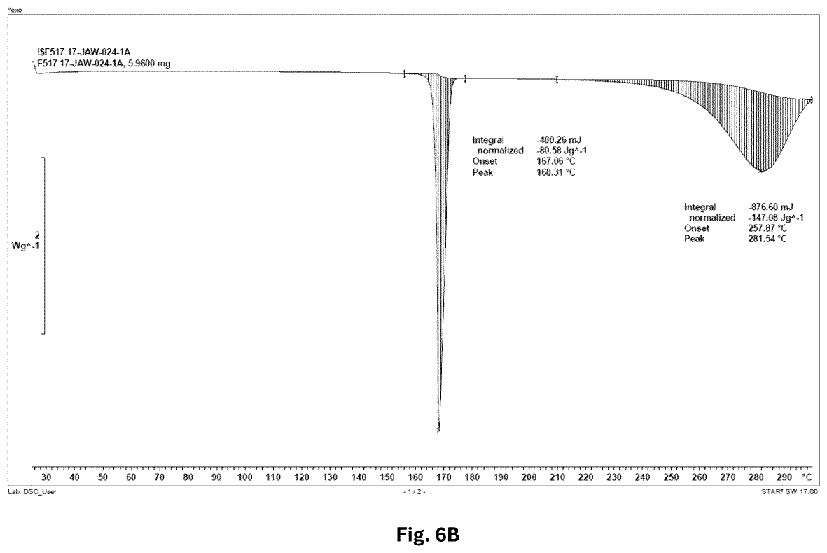

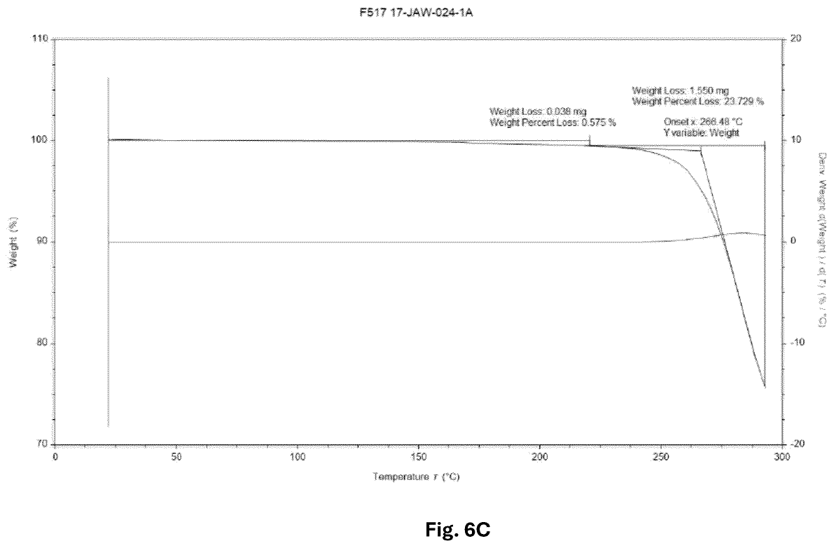

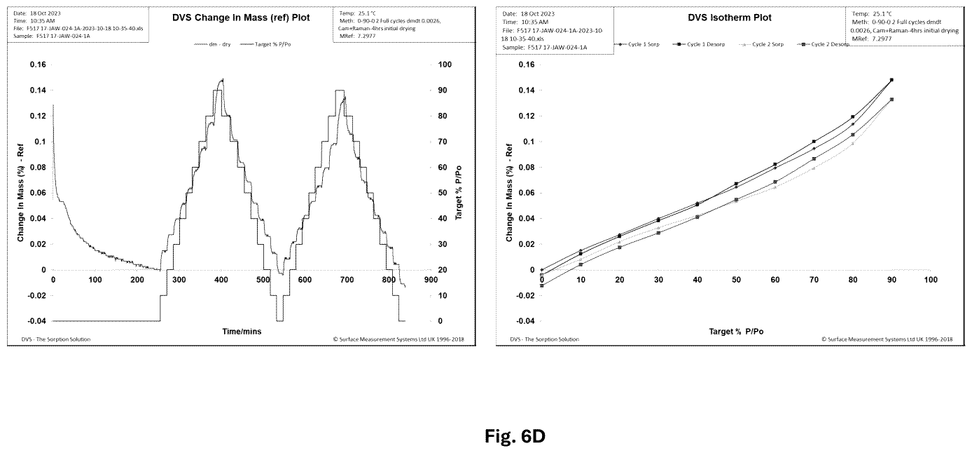

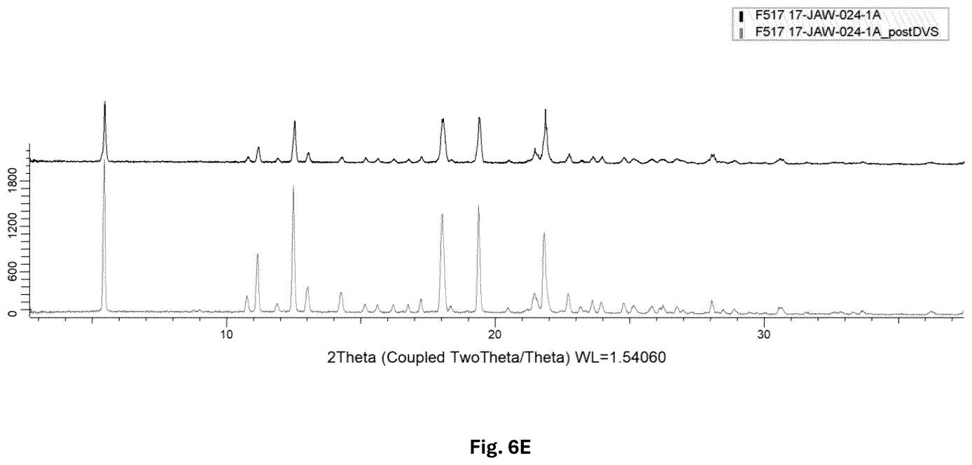

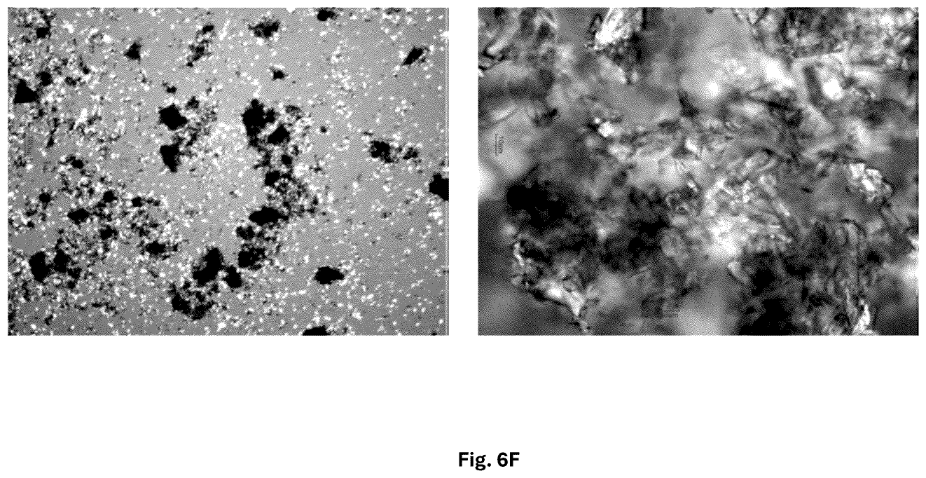

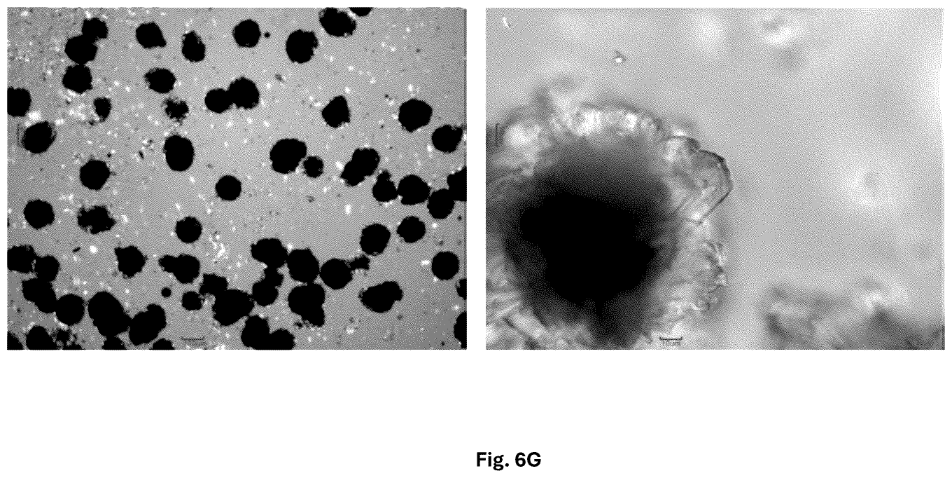

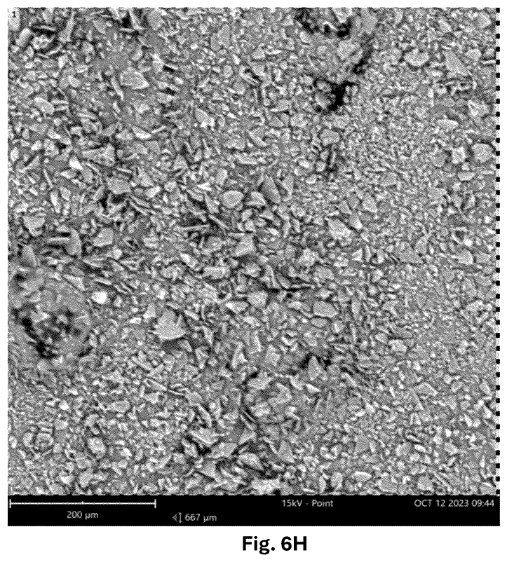

In some embodiments, the Compound 1 Form C freebase exhibits an XRPD that is substantially similar to FIG. 6A.

In some embodiments, the Compound 1 Form C freebase exhibits a DSC thermogram comprising a sharp endotherm that onsets at a temperature of 167° C. having a margin of error of ±10.0; ±5.0; ±4.0; ±3.0; ±2.0; ±1.0; or less. In some embodiments, the Compound 1 Form C freebase exhibits a DSC thermogram comprising an additional endotherm (broad) that onsets at a temperature of 258° C. with the margin of error of ±10.0; ±5.0; ±4.0; ±3.0; ±2.0; ±1.0; or less. In some embodiments, the Compound 1 Form C freebase exhibits a DSC thermogram that is substantially similar to FIG. 6B.

In some embodiments, the Compound 1 Form C freebase exhibits a TGA thermogram that is substantially similar to FIG. 6C. In some embodiments, the TGA thermogram of the Compound 1 Form C freebase exhibits a weight loss of about 1-5% between 25° C. and 266° C. In some embodiments, the TGA thermogram of the Compound 1 Form C freebase exhibits a weight loss of about 1-2% between 25° C. and 266° C. In other embodiments, the TGA thermogram of the Compound 1 Form C freebase exhibits a weight loss of about 0.6% between 25° C. and 266° C. In some embodiments, the TGA thermogram of the Compound 1 Form C freebase exhibits an additional weight loss of about 20-30% that onsets at a temperature of 266±5° C. In further embodiments, the TGA thermogram of the Compound 1 Form C freebase exhibits an additional weight loss of about 23.7% that onsets at a temperature of about 266° C.

In some embodiments, the Compound 1 Form C freebase exhibits a DVS isotherm plot that is substantially similar to FIG. 6D. In some embodiments, the Compound 1 Form C freebase exhibits a mass increase of about 0.05-0.5% due to moisture intake at 80-90% relative humidity (RH). In some embodiments, the Compound 1 Form C freebase exhibits a mass increase of about 0.15% due to moisture intake at about 90% RH. In some embodiments, the Compound 1 Form C freebase exhibits a mass increase of about 0.01-0.1% due to moisture intake at 50-60% RH In some embodiments, the Compound 1 Form C freebase exhibits a mass increase of about 0.06% due to moisture intake at about 50% RH. In some embodiments, the Compound 1 Form C freebase exhibits a mass loss on drying of less than 0.5% at about 0% RH. In some embodiments, the Compound 1 Form C freebase exhibits a mass loss on drying of about 0.1% at about 0% RH.

In one embodiment, the Compound 1 Form C freebase has a purity of at least about 75%, at least about 80%, at least about 85%, at least about 90%, at least about 95%, at least about 97%, at least about 98%, or at least about 99%.

In one embodiment, the Compound 1 Form C freebase has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, the Compound 1 Form C freebase has a purity of about 75% to about 99%. In one embodiment, the Compound 1 Form C freebase has a purity of about 80% to about 99%. In one embodiment, the Compound 1 Form C freebase has a purity of about 85% to about 99%. In one embodiment, the Compound 1 Form C freebase has a purity of about 90% to about 99%. In one embodiment, the Compound 1 Form C freebase has a purity of about 95% to about 99%. In one embodiment, the Compound 1 Form C freebase has a purity of about 97.5% to about 99%.

In one embodiment, the specific solid form of the Compound 1 Form C freebase with high purity is crystalline Form C freebase. In some embodiments, the Compound 1 Form C freebase is prepared according to the procedure described in Example 6.

### Compound 1 Solvates and Hydrates

In some embodiments, the Compound 1 crystalline form or amorphous form disclosed herein is a solvate. In some embodiments, the Compound 1 crystalline form or amorphous form disclosed herein is a hydrate. In one embodiment, the crystalline form of the Compound 1 is a Compound 1 salt or cocrystal. In another embodiment, the Compound 1 crystalline form disclosed herein is a non-solvate (i.e., includes no water and/or solvent molecules in the lattice). In one embodiment, the Compound 1 crystalline form disclosed herein is anhydrous. In some embodiments, the Compound 1 Form C freebase is anhydrous. In some embodiments, the Compound 1 Form I oxalate salt or oxalic acid cocrystal is anhydrous.

### Amorphous Form

The present disclosure also relates to solid amorphous forms of Compound 1 or a pharmaceutically acceptable salt thereof. In one embodiment, the present disclosure relates to an amorphous form of Compound 1 or a pharmaceutically acceptable salt thereof that is non-solvated. In one embodiment, the amorphous form of Compound 1 or a pharmaceutically acceptable salt thereof is a solvate. In some embodiments, the amorphous form of Compound 1 or a pharmaceutically acceptable salt thereof is a hydrate. In some embodiments, the amorphous form of Compound 1 is a co-amorphous form.

### Pharmaceutical Compositions

The present invention further provides pharmaceutical compositions comprising (1) a crystalline form of Compound 1 (e.g., Compound 1 salt or cocrystal form); and (2) a pharmaceutically acceptable carrier or excipient. In some embodiments, the pharmaceutical composition comprises Compound 1 Form I oxalate salt or oxalic acid cocrystal. In some embodiments, the pharmaceutical composition comprises Compound 1 Form II bromide salt or hydrobromide cocrystal. In some embodiments, the pharmaceutical composition comprises Compound 1 Form III chloride salt or hydrochloride cocrystal. In some embodiments, the pharmaceutical composition comprises Compound 1 Form IV phosphate salt or phosphoric acid cocrystal. In some embodiments, the pharmaceutical composition comprises Compound 1 Form A freebase. In some embodiments, the pharmaceutical composition comprises Compound 1 Form C freebase. In some embodiments, the pharmaceutical composition comprises an amorphous form of Compound 1 or a pharmaceutically acceptable salt thereof. In some embodiments, the amorphous form of Compound 1 is a co-amorphous form disclosed herein. The excipients are added to the formulation for a variety of purposes.

The present invention also provides a pharmaceutical composition comprising (1) a mixture of crystalline forms of Compound 1 disclosed herein; and (2) a pharmaceutically acceptable carrier or excipient. In one embodiment, the pharmaceutical composition comprises a mixture of crystalline salt forms of Compound 1 disclosed herein. In one embodiment, the pharmaceutical composition comprises a mixture of Compound 1 cocrystals disclosed herein. In one embodiment, the pharmaceutical composition comprises a mixture of crystalline freebase forms of Compound 1 disclosed herein. In one embodiment, the pharmaceutical composition comprises a mixture of a crystalline freebase of Compound 1 disclosed herein and a crystalline Compound 1 salt or a Compound 1 cocrystal disclosed herein. In one embodiment, the pharmaceutical composition comprises a mixture of a crystalline form of Compound 1 and an amorphous form of Compound 1.

In some embodiments, Compound 1 Form I oxalate salt or oxalic acid cocrystal is present in greater than about 10%, about 20%, about 30%, about 40%, about 50%, about 60%, about 70%, about 80%, about 85%, about 90%, or about 95% by weight of the pharmaceutical composition. In some embodiments, Compound 1 Form I oxalate salt or oxalic acid cocrystal is present in greater than about 90%, about 91%, about 92%, about 93%, about 94%, about 95%, about 96%, about 97%, about 98%, about 99%, or about 99.5% by weight of the pharmaceutical composition.

In some embodiments, Compound 1 Form C freebase is present in greater than about 10%, about 20%, about 30%, about 40%, about 50%, about 60%, about 70%, about 80%, about 85%, about 90%, or about 95% by weight of the pharmaceutical composition. In some embodiments, Compound 1 Form C freebase is present in greater than about 90%, about 91%, about 92%, about 93%, about 94%, about 95%, about 96%, about 97%, about 98%, about 99%, or about 99.5% by weight of the pharmaceutical composition.

In some embodiments, the pharmaceutical composition is formulated for administration orally, nasally, transdermally, topically, pulmonary, inhalationally, buccally, sublingually, intraperitoneally, subcutaneously, intramuscularly, intravenously, rectally, intrapleurally, intrathecally, intraportally, or parenterally, or as an implant.

In some embodiments, the pharmaceutical composition comprises about 1 mg to about 1000 mg of a crystalline form of Compound 1 (e.g., Compound 1 Form I salt or cocrystal). In some embodiments, the pharmaceutical composition comprises about 10 mg to about 500 mg of a crystalline form of Compound 1 (e.g., Compound 1 Form I salt or cocrystal).

### Therapeutic Methods

The present disclosure further provides methods of treating or managing a disease or a disorder, e.g., a disease or disorder mediated by AAK1 activity, comprising administering to a subject in need thereof, a salt of Compound 1 disclosed herein, a crystalline form of Compound 1 disclosed herein, or polymorph form disclosed herein.

In some embodiments, the disease or a disorder mediated by AAK1 activity is a muscular dystrophy. Muscular dystrophies are genetic diseases characterized by progressive weakness and degeneration of the skeletal or voluntary muscles which control movement. The muscles of the heart and some other involuntary muscles are also affected in some forms of muscular dystrophy. In many cases, the histological picture shows variation in fiber size, muscle cell necrosis and regeneration, and often proliferation of connective and adipose tissue. In some embodiments, the muscular dystrophy is Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy. In some embodiments, the muscular dystrophy is DMD.

In some embodiments, the disease or disorder mediated by AAK1 activity is a neurodegenerative disease. In some embodiments, the disease or disorder mediated by AAK1 activity is diabetic neuropathy.

The present disclosure also provides method for increasing skeletal muscle tissue growth or regeneration in a subject, comprising administering to the subject a compound disclosed herein or a pharmaceutically acceptable salt thereof.

In some embodiments, the subject has a muscular dystrophy. In some embodiments, the muscular dystrophy is Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy. In some embodiments, the subject has DMD.

In specific embodiments, the present disclosure provides methods for treating or managing a muscular dystrophy, comprising administering to a subject in need thereof a compound disclosed herein or pharmaceutically acceptable salt thereof. In some embodiments, the muscular dystrophy is selected from the group consisting of Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) and a congenital muscular dystrophy. In specific embodiments, the muscular dystrophy is DMD.

In some embodiments, the skeletal muscle tissue of the subject is damaged or injured as a result of physical injury or accident, disease, gene mutation, infection, over-use, loss of blood circulation, muscle atrophy, muscle wasting, dystrophic muscle, or ageing.

In some embodiments, the present disclosure provides methods for increasing asymmetric cell division of skeletal muscle stem cells, comprising contacting the skeletal muscle stem cells with a compound disclosed herein or pharmaceutically acceptable salt thereof. In particular embodiments, the stem cells are muscle stem cells, retinal stem cells, neural stem cells, hematopoietic stem cells, intestinal stem cells, epidermal stem cells, or cancer or tumor stem cells. In certain embodiments, the stem cells are muscle stem cells or satellite cells. In particular embodiments of any of the methods disclosed herein, the contacting between the inhibitor and the cells occurs in vitro, in vivo, ex vivo, or in situ. In some embodiments, the cells are mammalian cells. In specific embodiments, the cells are human cells.

In some embodiments, skeletal muscle stem cells within the skeletal muscle tissue of the subject have reduced asymmetric cell division as compared to normal, healthy skeletal muscle stem cells. In certain embodiments, the skeletal muscle stem cells are damaged or injured skeletal muscle stem cells or are present within damaged or injured skeletal muscle tissue. In some embodiments, the muscle tissue is damaged or injured as a result of: physical injury or accident, disease, gene mutation, infection, over-use, loss of blood circulation, muscle atrophy, muscle wasting, dystrophic muscle, or ageing. In some embodiments, the skeletal muscle stem cells are diseased skeletal muscle stem cells comprising a mutation associated with a muscular dystrophy, optionally Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy.

In some embodiments, the compound or pharmaceutically acceptable salt thereof inhibits an activity of AAK1. In some embodiments, the compound or pharmaceutically acceptable salt thereof inhibits AAK1. In some embodiments of the present methods, the compound or pharmaceutically acceptable salt thereof is an AAK1 inhibitor, and the compound or pharmaceutically acceptable salt thereof does not substantially inhibit proliferation or cell cycle progression of the subject's skeletal muscle stem cells.

In some embodiments of the present methods, the compound or pharmaceutically acceptable salt thereof inhibits expression of AAK1, optionally by inhibiting transcription, translation, post-translational modification, or stability of the protein component, or the gene encoding the protein component.

In some embodiments of the present methods, the compound or pharmaceutically acceptable salt thereof binds to a polynucleotide sequence that regulates expression of AAK1. In some embodiments, the nucleotide sequence is present within the AAK1 gene.

In some embodiments of the present methods, the compound or pharmaceutically acceptable salt thereof binds to a polynucleotide sequence that regulates expression of AAK1. In some embodiments, the nucleotide sequence is present within the AAK1 gene. In some embodiments, the polynucleotide sequence is DNA or RNA.

In some embodiments of the present methods, the compound or pharmaceutically acceptable salt thereof is administered to the subject systemically or locally, optionally at a site of tissue damage or injury.

In some embodiments, the compound or pharmaceutically acceptable salt thereof is administered at a dose of about 0.01 mg/kg to about 300 mg/kg.

In some embodiments, the subject has a mutation of a dystrophin gene.

In some embodiments, the subject has damaged or injured skeletal muscle tissue.

In some embodiments, the subject is a mammal. In some embodiments, the subject is a human.

In some embodiments, the methods disclosed herein increase skeletal muscle tissue regeneration in the subject.

In some embodiments, not limited by mechanism, the methods disclosed herein relate to the preservation of, maintenance of, and/or regeneration of, muscle mass, connectivity, and/or function.

### NUMBERED EMBODIMENTS

1. An oxalate salt of Compound 1.
2. An oxalic acid cocrystal of Compound 1.
3. The oxalic acid cocrystal of embodiment 2, wherein at least 90% by weight of the cocrystal is a single crystalline form.
4. The oxalic acid cocrystal of embodiment 2 or 3, wherein the crystalline form is Compound 1 Form I.
5. The oxalic acid cocrystal of embodiment 4, wherein the Form I exhibits an XRPD pattern comprising one or more peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.
6. The oxalic acid cocrystal of embodiment 4, wherein the Form I exhibits an XRPD pattern comprising three or more peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.
7. The oxalic acid cocrystal of embodiment 4, wherein the Form I exhibits an XRPD pattern comprising five or more peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.
8. The oxalic acid cocrystal of embodiment 4, wherein the Form I exhibits an XRPD pattern comprising peaks at 10.29±0.2, 12.28±0.2, and 21.78±0.2 degrees two-theta.
9. The oxalic acid cocrystal of embodiment 4, wherein the Form I exhibits an XRPD pattern comprising peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.
10. The oxalic acid cocrystal of any one of embodiments 4-9, wherein the Form I exhibits an XRPD pattern substantially similar to FIG. 1A.
11. The oxalic acid cocrystal of any one of embodiments 4-10, wherein the Form I exhibits a differential scanning calorimetry (DSC) thermogram comprising an endothermic peak that onsets at 166±5.0° C.
12. The oxalic acid cocrystal of embodiment 11, wherein the Form I exhibits a differential scanning calorimetry (DSC) thermogram further comprising endothermic peaks that onsets at 180±5.0° C. and 245±5.0° C.
13. The oxalic acid cocrystal of any one of embodiments 4-12, wherein the Form I exhibits a DSC thermogram substantially similar to FIG. 1B.
14. The oxalic acid cocrystal of any one of embodiments 4-13, wherein the Form I exhibits a weight loss of about 15-25% that onsets at a temperature of 179±5.0° C. by thermogravimetric analysis.
15. The oxalic acid cocrystal of any one of embodiments 4-14, wherein the Form I exhibits a weight loss of about 15-25% that onsets at a temperature of 241±5.0° C. by thermogravimetric analysis.
16. The oxalic acid cocrystal of any one of embodiments 4-15, wherein the Form I exhibits a thermogravimetric analysis thermogram substantially similar to FIG. 1C.
17. The oxalic acid cocrystal of any one of embodiments 4-16, wherein the Form I exhibits a mass loss on drying of about 0.1-0.5% at about 0% relative humidity (RH) according to dynamic vapor sorption (DVS).
18. The oxalic acid cocrystal of any one of embodiments 4-17, wherein the Form I exhibits a mass increase for water uptake of about 0.1-0.5% at about 50% RH according to DVS.
19. The oxalic acid cocrystal of any one of embodiments 4-18, wherein the Form I exhibits a mass increase for water uptake of about 1-2% at about 90% RH according to DVS.
20. A bromide salt or hydrobromide cocrystal of Compound 1.
21. The bromide salt or hydrobromide cocrystal of embodiment 20, wherein the bromide salt is crystalline.
22. The hydrobromide or cocrystal of embodiment 20 or 21, wherein at least 90% by weight of the salt or cocrystal is a single crystalline form.
23. The bromide salt or hydrobromide cocrystal of any one of embodiments 20-22, wherein the crystalline form is Form II.
24. The bromide salt or hydrobromide cocrystal of embodiment 23, wherein the Form II exhibits an XRPD pattern comprising one or more peaks at 3.74±0.2, 7.48±0.2, 8.84±0.2, 10.97±0.2, 11.23±0.2, 13.81±0.2, 15.00±0.2, 16.49±0.2, 17.05±0.2, 17.73±0.2, 18.78±0.2, 20.45±0.2, 21.46±0.2, 21.96±0.2, 24.52±0.2, 24.80±0.2, 25.66±0.2, 26.73±0.2, 28.85±0.2, 34.21±0.2, and 36.48±0.2 degrees two-theta degrees two-theta.
25. The bromide salt or hydrobromide cocrystal of embodiment 23, wherein the Form II exhibits an XRPD pattern comprising three or more peaks at 3.74±0.2, 7.48±0.2, 8.84±0.2, 10.97±0.2, 11.23±0.2, 13.81±0.2, 15.00±0.2, 16.49±0.2, 17.05±0.2, 17.73±0.2, 18.78±0.2, 20.45±0.2, 21.46±0.2, 21.96±0.2, 24.52±0.2, 24.80±0.2, 25.66±0.2, 26.73±0.2, 28.85±0.2, 34.21±0.2, and 36.48±0.2 degrees two-theta.
26. The bromide salt or hydrobromide cocrystal of embodiment 23, wherein the Form II exhibits an XRPD pattern comprising five or more peaks at 3.74±0.2, 7.48±0.2, 8.84±0.2, 10.97±0.2, 11.23±0.2, 13.81±0.2, 15.00±0.2, 16.49±0.2, 17.05±0.2, 17.73±0.2, 18.78±0.2, 20.45±0.2, 21.46±0.2, 21.96±0.2, 24.52±0.2, 24.80±0.2, 25.66±0.2, 26.73±0.2, 28.85±0.2, 34.21±0.2, and 36.48±0.2 degrees two-theta.
27. The bromide salt or hydrobromide cocrystal of embodiment 23, wherein the Form II exhibits an XRPD pattern comprising peaks at 11.23±0.2, 20.45±0.2, 24.80±0.2, and 25.66±0.2 degrees two-theta.
28. The bromide salt or hydrobromide cocrystal of embodiment 23, wherein the Form II exhibits an XRPD pattern comprising peaks at 7.48±0.2, 10.97±0.2, 11.23±0.2, 15.00±0.2, 17.73±0.2, 18.78±0.2, 20.45±0.2, 21.46±0.2, 24.52±0.2, 24.80±0.2, and 25.66±0.2 degrees two-theta.
29. The bromide salt or hydrobromide cocrystal of any one of embodiments 23-28, wherein the Form II exhibits an XRPD pattern substantially similar to FIG. 2A.
30. The bromide salt or hydrobromide cocrystal of any one of embodiments 23-29, wherein the Form II exhibits a differential scanning calorimetry (DSC) thermogram comprising an endothermic peak that onsets at a temperature of 53±5.0° C. and is centered at 89±5.0° C.
31. The bromide salt or hydrobromide cocrystal of embodiment 30, wherein the Form II exhibits a differential scanning calorimetry thermogram further comprising endothermic peaks that onset at temperatures of 219±5.0° C. and 234±5.0° C.
32. The bromide salt or hydrobromide cocrystal of any one of embodiments 23-31, wherein the Form II exhibits a DSC thermogram substantially similar to FIG. 2B.
33. The bromide salt or hydrobromide cocrystal of any one of embodiments 23-32, wherein the Form II exhibits a weight loss of about 1-10% between 25° C. and 235° C. by thermogravimetric analysis.
34. The bromide salt or hydrobromide cocrystal of any one of embodiments 23-32, wherein the Form II exhibits a secondary weight loss of about 5-15% that onsets at a temperature of 235±5.0° C. by thermogravimetric analysis.
35. The bromide salt or hydrobromide cocrystal of any one of embodiments 23-34, wherein the Form II exhibits a thermogravimetric analysis thermogram substantially similar to FIG. 2C.
36. The bromide salt or hydrobromide cocrystal of any one of embodiments 23-35, wherein the Form II exhibits a mass loss on drying of about 1-3% at about 0% relative humidity (RH) according to dynamic vapor sorption (DVS).
37. The bromide salt or hydrobromide cocrystal of any one of embodiments 23-36, wherein the Form II exhibits a mass increase for water uptake of about 1-5% at about 50% RH according to DVS.
38. The bromide salt or hydrobromide cocrystal of any one of embodiments 23-37, wherein the Form II exhibits a mass increase for water uptake of about 10-15% at about 90% RH according to DVS.
39. A chloride salt or hydrochloride cocrystal of Compound 1.
40. The chloride salt or hydrochloride cocrystal of embodiment 39, wherein the chloride salt is crystalline.
41. The chloride salt or hydrochloride cocrystal of embodiment 39 or 40, wherein at least 90% by weight of the salt or cocrystal is a single crystalline form.
42. The chloride salt or hydrochloride cocrystal of any one of embodiments 39-41, wherein the crystalline form is Form III.
43. The chloride salt or hydrochloride cocrystal of embodiment 42, wherein the Form III exhibits an XRPD pattern comprising one or more peaks at about one or more peaks at 7.37±0.2, 8.97±0.2, 11.09±0.2, 13.89±0.2, 14.83±0.2, 16.28±0.2, 18.06±0.2, 20.44±0.2, 20.53±0.2, 24.84±0.2, 25.18±0.2, 25.22±0.2, and 26.14±0.2 degrees two-theta degrees two-theta.
44. The chloride salt or hydrochloride cocrystal of embodiment 42, wherein the Form III exhibits an XRPD pattern comprising three or more peaks at 7.37±0.2, 8.97±0.2, 11.09±0.2, 13.89±0.2, 14.83±0.2, 16.28±0.2, 18.06±0.2, 20.44±0.2, 20.53±0.2, 24.84±0.2, 25.18±0.2, 25.22±0.2, and 26.14±0.2 degrees two-theta.
45. The chloride salt or hydrochloride cocrystal of embodiment 42, wherein the Form III exhibits an XRPD pattern comprising five or more peaks at 7.37±0.2, 8.97±0.2, 11.09±0.2, 13.89±0.2, 14.83±0.2, 16.28±0.2, 18.06±0.2, 20.44±0.2, 20.53±0.2, 24.84±0.2, 25.18±0.2, 25.22±0.2, and 26.14±0.2 degrees two-theta.
46. The chloride salt or hydrochloride cocrystal of embodiment 42, wherein the Form III exhibits an XRPD pattern comprising peaks at 11.09±0.2, 13.89±0.2, 25.18±0.2, and 25.22±0.2 degrees two-theta.
47. The chloride salt or hydrochloride cocrystal of embodiment 42, wherein the Form III exhibits an XRPD pattern comprising peaks at 8.97±0.2, 11.09±0.2, 13.89±0.2, 14.83±0.2, 20.53±0.2, 25.18±0.2, 25.22±0.2, and 26.14±0.2 degrees two-theta.
48. The chloride salt or hydrochloride cocrystal of any one of embodiments 42-47, wherein the Form III exhibits an XRPD pattern substantially similar to FIG. 3A.
49. The chloride salt or hydrochloride cocrystal of any one of embodiments 42-48, wherein the Form III exhibits a differential scanning calorimetry (DSC) thermogram comprising an endothermic peak that onsets at a temperature of 42±5.0° C.
50. The chloride salt or hydrochloride cocrystal of embodiment 49, wherein the Form III exhibits a differential scanning calorimetry thermogram further comprising an endothermic peak that onsets at a temperature of 192±5.0° C.
51. The chloride salt or hydrochloride cocrystal of any one of embodiments 42-50, wherein the Form III exhibits a DSC thermogram substantially similar to FIG. 3B.
52. The chloride salt or hydrochloride cocrystal of any one of embodiments 42-51, wherein the Form III exhibits a weight loss of about 1-5% between 25° C. and 85° C. by thermogravimetric analysis.
53. The chloride salt or hydrochloride cocrystal of any one of embodiments 42-51, wherein the Form III exhibits additional weight loss events of about 5-10% centered on a temperature of 187±5.0° C. and about 15-25% centered on a temperature of 249±5.0° C. by thermogravimetric analysis.
54. The chloride salt or hydrochloride cocrystal of any one of embodiments 42-53, wherein the Form III exhibits a thermogravimetric analysis thermogram substantially similar to FIG. 3C.
55. The chloride salt or hydrochloride cocrystal of any one of embodiments 42-54, wherein the Form III exhibits a mass loss on drying of about 0.5-1.5% at about 0% relative humidity (RH) according to dynamic vapor sorption (DVS).
56. The chloride salt or hydrochloride cocrystal of any one of embodiments 42-55, wherein the Form III exhibits a mass increase for water uptake of about 3-7% at about 50% RH according to DVS.
57. The chloride salt or hydrochloride cocrystal of any one of embodiments 42-56, wherein the Form III exhibits a mass increase for water uptake of about 17-21% at about 90% RH according to DVS.
58. A phosphate salt or phosphoric acid cocrystal of Compound 1.
59. The phosphate salt or phosphoric acid cocrystal of embodiment 58, wherein the phosphate salt is crystalline.
60. The phosphate salt or phosphoric acid cocrystal of embodiment 58 or 59, wherein at least 90% by weight of the salt or cocrystal is a single crystalline form.
61. The phosphate salt or phosphoric acid cocrystal of any one of embodiments 58-60, wherein the crystalline form is Form IV.
62. The phosphate salt or phosphoric acid cocrystal of embodiment 61, wherein the Form IV exhibits an XRPD pattern comprising one or more peaks at about one or more peaks at 3.66±0.2, 6.26±0.2, 7.34±0.2, 11.02±0.2, 12.56±0.2, 14.15±0.2, 14.39±0.2, 14.99±0.2, 15.32±0.2, 16.19±0.2, 17.72±0.2, 18.38±0.2, 18.86±0.2, 19.50±0.2, 19.77±0.2, 20.76±0.2, 21.08±0.2, 21.29±0.2, 22.08±0.2, 22.33±0.2, 22.76±0.2, 22.83±0.2, 23.14±0.2, 23.39±0.2, 23.68±0.2, 24.14±0.2, 24.52±0.2, 26.64±0.2, and 28.76±0.2 degrees two-theta degrees two-theta.
63. The phosphate salt or phosphoric acid cocrystal of embodiment 61, wherein the Form IV exhibits an XRPD pattern comprising three or more peaks at 3.66±0.2, 6.26±0.2, 7.34±0.2, 11.02±0.2, 12.56±0.2, 14.15±0.2, 14.39±0.2, 14.99±0.2, 15.32±0.2, 16.19±0.2, 17.72±0.2, 18.38±0.2, 18.86±0.2, 19.50±0.2, 19.77±0.2, 20.76±0.2, 21.08±0.2, 21.29±0.2, 22.08±0.2, 22.33±0.2, 22.76±0.2, 22.83±0.2, 23.14±0.2, 23.39±0.2, 23.68±0.2, 24.14±0.2, 24.52±0.2, 26.64±0.2, and 28.76±0.2 degrees two-theta.
64. The phosphate salt or phosphoric acid cocrystal of embodiment 61, wherein the Form IV exhibits an XRPD pattern comprising five or more peaks at 3.66±0.2, 6.26±0.2, 7.34±0.2, 11.02±0.2, 12.56±0.2, 14.15±0.2, 14.39±0.2, 14.99±0.2, 15.32±0.2, 16.19±0.2, 17.72±0.2, 18.38±0.2, 18.86±0.2, 19.50±0.2, 19.77±0.2, 20.76±0.2, 21.08±0.2, 21.29±0.2, 22.08±0.2, 22.33±0.2, 22.76±0.2, 22.83±0.2, 23.14±0.2, 23.39±0.2, 23.68±0.2, 24.14±0.2, 24.52±0.2, 26.64±0.2, and 28.76±0.2 degrees two-theta.
65. The phosphate salt or phosphoric acid cocrystal of embodiment 61, wherein the Form IV exhibits an XRPD pattern comprising peaks at 7.34±0.2, 11.02±0.2, 17.72±0.2, 19.77±0.2, 20.76±0.2, 21.29±0.2, 22.76±0.2, 22.83±0.2, 23.14±0.2, 24.14±0.2, and 24.52±0.2 degrees two-theta.
66. The phosphate salt or phosphoric acid cocrystal of embodiment 61, wherein the Form IV exhibits an XRPD pattern comprising peaks at 7.34±0.2, 11.02±0.2, 17.72±0.2, 18.38±0.2, 18.86±0.2, 19.50±0.2, 19.77±0.2, 20.76±0.2, 21.29±0.2, 22.08±0.2, 22.76±0.2, 22.83±0.2, 23.14±0.2, 23.39±0.2, 23.68±0.2, 24.14±0.2, and 24.52±0.2 degrees two-theta.
67. The phosphate salt or phosphoric acid cocrystal of any one of embodiments 61-66, wherein the Form IV exhibits an XRPD pattern substantially similar to FIG. 4A.
68. The phosphate salt or phosphoric acid cocrystal of any one of embodiments 61-67, wherein the Form IV exhibits a differential scanning calorimetry (DSC) thermogram comprising an endothermic peak that onsets at a temperature of 40±5.0° C.
69. The phosphate salt or phosphoric acid cocrystal of embodiment 68, wherein the Form IV exhibits a differential scanning calorimetry thermogram further comprising endothermic peaks that onset at about 154±5.0° C. and 221±5.0° C.
70. The phosphate salt or phosphoric acid cocrystal of any one of embodiments 61-69, wherein the Form IV exhibits a DSC thermogram substantially similar to FIG. 4B.
71. The phosphate salt or phosphoric acid cocrystal of any one of embodiments 61-70, wherein the Form IV exhibits a weight loss of about 1-10% that onsets at a temperature of 73±5.0° C. by thermogravimetric analysis.
72. The phosphate salt or phosphoric acid cocrystal of any one of embodiments 61-70, wherein the Form IV exhibits an additional weight loss event of about 5-15% that onsets at a temperature of 194±5.0° C. by thermogravimetric analysis.
73. The phosphate salt or phosphoric acid cocrystal of any one of embodiments 61-72, wherein the Form IV exhibits a thermogravimetric analysis thermogram substantially similar to FIG. 4C.
74. The phosphate salt or phosphoric acid cocrystal of any one of embodiments 61-73, wherein the Form IV exhibits a mass loss on drying of about 3-7% at about 0% relative humidity (RH) according to dynamic vapor sorption (DVS).
75. The phosphate salt or phosphoric acid cocrystal of any one of embodiments 61-74, wherein the Form IV exhibits a mass increase for water uptake of about 3-7% at about 50% RH according to DVS.
76. The phosphate salt or phosphoric acid cocrystal of any one of embodiments 61-75, wherein the Form IV exhibits a mass increase for water uptake of about 5-10% at about 90% RH according to DVS.
77. A crystalline freebase of Compound 1.
78. The crystalline freebase of embodiment 77, wherein at least 90% by weight of the freebase is crystalline.
79. The crystalline freebase of embodiment 77 or 78, wherein at least 90% by weight of the freebase is a single crystalline form.
80. The crystalline freebase of any one of embodiments 77-79, wherein the crystalline form is Form A.
81. The crystalline freebase of embodiment 80, wherein the Form A exhibits an XRPD pattern comprising one or more peaks at about one or more peaks at 6.45±0.2, 8.71±0.2, 11.02±0.2, 11.54±0.2, 12.11±0.2, 12.94±0.2, 14.63±0.2, 15.73±0.2, 16.80±0.2, 17.84±0.2, 18.44±0.2, 19.36±0.2, 19.77±0.2, 20.38±0.2, 20.54±0.2, 20.81±0.2, 21.10±0.2, 22.27±0.2, 27.70±0.2, 22.97±0.2, 24.48±0.2, 25.63±0.2, 25.92±0.2, 27.42±0.2, 27.91±0.2, 29.54±0.2, and 30.12±0.2 degrees two-theta degrees two-theta.
82. The crystalline freebase of embodiment 80, wherein the Form A exhibits an XRPD pattern comprising three or more peaks at 6.45±0.2, 8.71±0.2, 11.02±0.2, 11.54±0.2, 12.11±0.2, 12.94±0.2, 14.63±0.2, 15.73±0.2, 16.80±0.2, 17.84±0.2, 18.44±0.2, 19.36±0.2, 19.77±0.2, 20.38±0.2, 20.54±0.2, 20.81±0.2, 21.10±0.2, 22.27±0.2, 27.70±0.2, 22.97±0.2, 24.48±0.2, 25.63±0.2, 25.92±0.2, 27.42±0.2, 27.91±0.2, 29.54±0.2, and 30.12±0.2 degrees two-theta.
83. The crystalline freebase of embodiment 80, wherein the Form A exhibits an XRPD pattern comprising five or more peaks at 6.45±0.2, 8.71±0.2, 11.02±0.2, 11.54±0.2, 12.11±0.2, 12.94±0.2, 14.63±0.2, 15.73±0.2, 16.80±0.2, 17.84±0.2, 18.44±0.2, 19.36±0.2, 19.77±0.2, 20.38±0.2, 20.54±0.2, 20.81±0.2, 21.10±0.2, 22.27±0.2, 27.70±0.2, 22.97±0.2, 24.48±0.2, 25.63±0.2, 25.92±0.2, 27.42±0.2, 27.91±0.2, 29.54±0.2, and 30.12±0.2 degrees two-theta.
84. The crystalline freebase of embodiment 80, wherein the Form A exhibits an XRPD pattern comprising peaks at 8.71±0.2, 11.02±0.2, 11.54±0.2, 12.94±0.2±0.2, 14.63±0.2, 18.44±0.2, 19.36±0.2, 19.77±0.2, 20.38±0.2, 20.54±0.2, 22.27±0.2, 22.97±0.2, 24.48±0.2, and 25.63±0.2 degrees two-theta.
85. The crystalline freebase of embodiment 80, wherein the Form A exhibits an XRPD pattern comprising peaks at 6.45±0.2, 8.71±0.2, 11.02±0.2, 11.54±0.2, 12.11±0.2, 12.94±0.2±0.2, 14.63±0.2, 17.84±0.2, 18.44±0.2, 19.36±0.2, 19.77±0.2, 20.38±0.2, 20.54±0.2, 20.81±0.2, 21.10±0.2, 22.27±0.2, 27.70±0.2, 22.97±0.2, 24.48±0.2, 25.63±0.2, and 27.42±0.2 degrees two-theta.
86. The crystalline freebase of any one of embodiments 80-85, wherein the Form A exhibits an XRPD pattern substantially similar to FIG. 5A.
87. The crystalline freebase of any one of embodiments 80-86, wherein the Form A exhibits a differential scanning calorimetry (DSC) thermogram comprising an endothermic peak that onsets at a temperature of 80±5.0° C.
88. The crystalline freebase of embodiment 87, wherein the Form A exhibits a differential scanning calorimetry thermogram further comprising endothermic peaks that onset temperatures of 146±5.0° C. and 181±5.0° C.
89. The crystalline freebase of any one of embodiments 80-88, wherein the Form A exhibits a DSC thermogram substantially similar to FIG. 5B.
90. The crystalline freebase of any one of embodiments 80-89, wherein the Form A exhibits a weight loss of about 3-7% that onsets at a temperature of 56±5.0° C. by thermogravimetric analysis.
91. The crystalline freebase of any one of embodiments 80-89, wherein the Form A exhibits an additional weight loss of about 20-30% that onsets at a temperature of 217±5.0° C. by thermogravimetric analysis.
92. The crystalline freebase of any one of embodiments 80-91, wherein the Form A exhibits a thermogravimetric analysis thermogram substantially similar to FIG. 5C.
93. The crystalline freebase of any one of embodiments 80-92, wherein the Form A exhibits a mass loss on drying of about 3-7% at about 0% relative humidity (RH) according to dynamic vapor sorption (DVS).
94. The crystalline freebase of any one of embodiments 80-93, wherein the Form A exhibits a mass increase for water uptake of about 3-7% at about 50% RH according to DVS.
95. The crystalline freebase of any one of embodiments 80-94, wherein the Form A exhibits a mass increase for water uptake of about 10-20% at about 90% RH according to DVS.
96. The crystalline freebase of any one of embodiments 77-79, wherein the crystalline form is Form C.
97. The crystalline freebase of embodiment 96, wherein the Form C exhibits an XRPD pattern comprising one or more peaks at 5.48±0.2, 11.20±0.2, 12.55±0.2, 13.06±0.2, 18.05±0.2, 19.41±0.2, 21.48±0.2, 21.58±0.2, 21.87±0.2, 22.75±0.2, 23.65±0.2, 23.99±0.2, 24.79±0.2, 28.05±0.2, and 28.13±0.2 degrees two-theta.
98. The crystalline freebase of embodiment 96, wherein the Form C exhibits an XRPD pattern comprising three or more peaks at 5.48±0.2, 11.20±0.2, 12.55±0.2, 13.06±0.2, 18.05±0.2, 19.41±0.2, 21.48±0.2, 21.58±0.2, 21.87±0.2, 22.75±0.2, 23.65±0.2, 23.99±0.2, 24.79±0.2, 28.05±0.2, and 28.13±0.2 degrees two-theta.
99. The crystalline freebase of embodiment 96, wherein the Form C exhibits an XRPD pattern comprising five or more peaks at 5.48±0.2, 11.20±0.2, 12.55±0.2, 13.06±0.2, 18.05±0.2, 19.41±0.2, 21.48±0.2, 21.58±0.2, 21.87±0.2, 22.75±0.2, 23.65±0.2, 23.99±0.2, 24.79±0.2, 28.05±0.2, and 28.13±0.2 degrees two-theta.
100. The crystalline freebase of embodiment 96, wherein the Form C exhibits an XRPD pattern comprising peaks at 5.48±0.2, 12.55±0.2, 18.05±0.2, 19.41±0.2, and 21.87±0.2 degrees two-theta.
101. The crystalline freebase of embodiment 96, wherein the Form C exhibits an XRPD pattern comprising peaks at 5.48±0.2, 11.20±0.2, 12.55±0.2, 18.05±0.2, 19.41±0.2, 21.48±0.2, 21.87±0.2, 22.75±0.2, and 28.05±0.2 degrees two-theta.
102. The crystalline freebase of any one of embodiments 96-101, wherein the Form C exhibits an XRPD pattern substantially similar to FIG. 6A.
103. The crystalline freebase of any one of embodiments 96-102, wherein the Form C exhibits a differential scanning calorimetry (DSC) thermogram comprising an endothermic peak that onsets at a temperature of 167±5.0° C.
104. The crystalline freebase of embodiment 103, wherein the Form C exhibits a differential scanning calorimetry thermogram further comprising an endothermic peak that onsets at a temperature of 258±5.0° C.
105. The crystalline freebase of any one of embodiments 96-104, wherein the Form C exhibits a DSC thermogram substantially similar to FIG. 6B.
106. The crystalline freebase of any one of embodiments 96-105, wherein the Form C exhibits a weight loss of about 1-2% between about 25° C. and 266° C. by thermogravimetric analysis.
107. The crystalline freebase of any one of embodiments 96-105, wherein the Form C exhibits an additional weight loss of about 20-30% that onsets at a temperature of 266±5.0° C. by thermogravimetric analysis.
108. The crystalline freebase of any one of embodiments 96-107, wherein the Form C exhibits a thermogravimetric analysis thermogram substantially similar to FIG. 6C.
109. The crystalline freebase of any one of embodiments 96-108, wherein the Form C exhibits a mass loss on drying of about 0.1% at about 0% relative humidity (RH) according to dynamic vapor sorption (DVS).
110. The crystalline freebase of any one of embodiments 96-109, wherein the Form C exhibits a mass increase for water uptake of about 0.01-0.1% at about 50% RH according to DVS.
111. The crystalline freebase of any one of embodiments 96-110, wherein the Form C exhibits a mass increase for water uptake of about 0.05-0.25% at about 90% RH according to DVS.
112. A pharmaceutical composition comprising the Compound 1 oxalate salt or oxalic acid cocrystal of any one of embodiments 1-19, and a pharmaceutically acceptable carrier or excipient.
113. A pharmaceutical composition comprising the Compound 1 bromide salt or hydrobromide cocrystal of any one of embodiments 20-38, and a pharmaceutically acceptable carrier or excipient.
114. A pharmaceutical composition comprising the Compound 1 chloride salt or hydrobromide cocrystal of any one of embodiments 39-57 and a pharmaceutically acceptable carrier or excipient.
115. A pharmaceutical composition comprising the Compound 1 phosphate salt or phosphoric acid cocrystal of any one of embodiments 58-76, and a pharmaceutically acceptable carrier or excipient.
116. A pharmaceutical composition comprising the Compound 1 freebase of any one of embodiments 77-111, and a pharmaceutically acceptable carrier or excipient.
117. A method for treating or managing a disease or a disorder mediated by AAK1 activity, comprising administering to a subject in need thereof, the pharmaceutical composition of any one of embodiments 112-116.
118. The method of embodiment 117, wherein the disease or a disorder mediated by AAK1 activity is a muscular dystrophy.
119. The method of embodiment 118, wherein the muscular dystrophy is Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy.
120. The method of embodiment any one of embodiments 117-119, wherein the subject has a mutation of a dystrophin gene.
121. A method for increasing skeletal muscle tissue growth or regeneration in a subject, comprising administering to the subject the pharmaceutical composition of any one of embodiments 112-116.
122. The method of embodiment 121, wherein the subject has damaged or injured skeletal muscle tissue.
123. The method of embodiment 122, wherein the skeletal muscle tissue is damaged or injured as a result of physical injury or accident, disease, gene mutation, infection, over-use, loss of blood circulation, muscle atrophy, muscle wasting, dystrophic muscle, or ageing.
124. The method of any one of embodiments 121-123, wherein the subject has a muscular dystrophy, optionally Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy.
125. The method of any one of embodiments 121-124, wherein the subject has a mutation of a dystrophin gene.
126. The method of any one of embodiments 121-125, wherein skeletal muscle stem cells within the skeletal muscle tissue have reduced asymmetric cell division as compared to normal, healthy skeletal muscle stem cells.
127. The method of any one of embodiments 121-126, wherein the method increases skeletal muscle tissue regeneration in the subject.
128. A method of treating a muscular dystrophy in a subject in need thereof, comprising administering the pharmaceutical composition of any one of embodiments 112-116 to the subject.
129. The method of embodiment 128, wherein the muscular dystrophy is selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) and a congenital muscular dystrophy.
130. The method of embodiment 128 or 129, wherein the subject has a mutation of a dystrophin gene.
131. The method of any one of embodiments 128-130, wherein the subject has damaged or injured skeletal muscle tissue.
132. The method of embodiment 131, wherein skeletal muscle stem cells within the skeletal muscle tissue have reduced asymmetric cell division as compared to normal, healthy skeletal muscle stem cells.
133. The method of any one of embodiments 128-132, wherein the method increases skeletal muscle tissue regeneration in the subject.

Having now generally described the invention, the same will be more readily understood through reference to the following examples, which are provided by way of illustration and are not intended to be limiting of the present invention.

### Examples

The disclosure now being generally described, it will be more readily understood by reference to the following examples which are included merely for purposes of illustration of certain aspects and embodiments of the present invention, and are not intended to limit the invention.

### Abbreviations

| | |
|---|---|
| API | Active Pharmaceutical Ingredient |
| ATR | Attenuated Total Reflectance |
| cGMP | Current Good Manufacturing Practices |
| DVS | Dynamic Vapor Sorption |
| DSC | Differential Scanning Calorimetry |
| EDS | Energy Dispersive X-ray spectroscopy |
| EtOAc | Ethyl Acetate |
| FTIR | Fourier-transform Infrared Spectroscopy |
| IPA | Isopropyl alcohol |
| iPrOAc | Isopropyl acetate |
| MeOH | Methanol |
| MTBE | Methyl Tert-butyl Ether |
| PLM | Polarized Light Microscopy |
| XRPD | Powder X-Ray Diffraction |
| rpm | Rotations per minute |
| RH | Relative Humidity |
| SEM | Scanning Electron Microscopy |
| SSRD | Solid State Research and Development |
| TGA | Thermogravimetric Analysis |

### General Procedures and Analytical Methods

XRPD Analysis: XRPD diffractograms were collected with an X-ray diffractometer (Bruker, D8 Discover). Samples were prepared on a zero-background silicon wafer by gently pressing onto the flat surface. The parameters of XRPD diffraction are provided in Table.

TABLE 7
Parameters Used for XRPD Analysis

| Parameter | Value |
|---|---|
| Current | 1.0 mA |
| Voltage | 50.0 kV |
| Source type | Cu K_{α}, point focus |
| Source wavelength | 1.54060 Å |
| Detector Type | Eiger 2R-500K 2-Dimensional |
| Scan type | Coupled TwoTheta/Theta |
| Geometry | Bragg-Brentano |
| Scan mode | Still, step |
| Collection time | 180 seconds |
| 2Theta Range | 5-20°2θ |
| Detector distance | 290 mm |
| Divergence slit width (mm) | 0.6 |

TGA Analysis: TGA analysis was performed using a TA Instrument.

TGA sample preparation: A solid sample (between 5-10 mg) was added to a tared Aluminum pan and pierced with a needle to allow for the outflow of produced gases. The sample was loaded into the instrument and kept under a flow of N₂ (60 cm³/min), and the mass continuously recorded. The sample was heated from 20° C. to 300° C. at a rate of 10° C./min. A summary of the operating parameters is provided in Table 8.

TABLE 8
Parameters of TGA Testing

| | |
|---|---|
| Instrument | TA, Discovery TGA 55 |
| Sample pan | Aluminum, open |
| Temperature range | 20-300° C. |
| Heating rate | 10° C./min |
| Purge gas | N₂ |
| Flow rate | 60 cm³/min |

DSC Analysis: DSC analysis was performed with a TA Instrument.

DSC sample preparation: A solid sample (between 5-10 mg) was added to a tared Aluminum pan and pierced with a needle to allow for the outflow of produced gases. The sample was loaded into the instrument alongside an empty reference pan and kept under a flow of N₂ (60 cm³/min). The sample was heated from 20° C. to 300° C. at a rate of 10° C./min. The difference in energy required to heat the sample compared to the empty reference pan is continuously recorded. A summary of the operating parameters is provided in Table 9.

TABLE 9
Parameters of DSC Analysis

| | |
|---|---|
| Instrument | TA, Discovery DSC 250 |
| Sample pan | Aluminum, pin-holed |
| Temperature range | 20-300° C. |
| Heating rate | 10° C./min |
| Purge gas | N₂ |
| Flow rate | 60 cm³/min |

DVS Analysis: Moisture sorption/desorption data were collected on a DVS Intrinsic. Solid sample was loaded on a quartz pan and placed in the instrument for analysis. A matched, empty reference pan was placed in an adjacent chamber and exposed to identical conditions. Two cycles of sorption and desorption were carried out at 25° C. (0 to 90% RH). Each step in RH was held until the derivative of mass with respect to time reached 0.009%/min or less. The maximum duration at each RH was 300 minutes. The anhydrate and hydrate were analyzed with the setting parameters in Table.

TABLE 10
Parameters of DVS Analysis for Anhydrates

| | |
|---|---|
| Instrument | SMS, DVS Intrinsic |
| dm/dt | 0.009%/min |
| Sample size | 5-25 mg |
| Drying/Measurement temperature | 25° C. |
| Cycle | |
| dm/dt stability duration | |
| Save data rate | |
| Gas and Total flow rate | |
| Post experiment total flow | |
| RH step size | 10% RH |
| Method | |

### Example A. Polymorph Screening Study

Three crystallization techniques were used to study the polymorphism of Compound 1 with the selected solvents
1. Slurry: Compound 1 solids (97.6% pure) and solvents were combined in a crystallizer and stirred at 45° C. for 21 hours
2. Cooling: A portion of filtered solution from the slurry crystallizer was cooled to 0° C. (cooling rate=1.0° C./min) to crystallize solids out of the solution. The filtration of the slurry solution was necessary to remove undissolved solids that may influence the solid form by acting as seed crystals.
3. Antisolvent Addition: A portion of filtered solution from the slurry crystallizer was added to a crystallizer pre-loaded with anti-solvents in an attempt to crystallize solids out of the solution. The mixtures of solvents and antisolvents were cooled to 0° C. (cooling rate=1.0° C./min). The filtration of the slurry solution was necessary to remove undissolved solids that may influence the solid form by acting as seed crystals.

### Experimental Procedure and Observations:

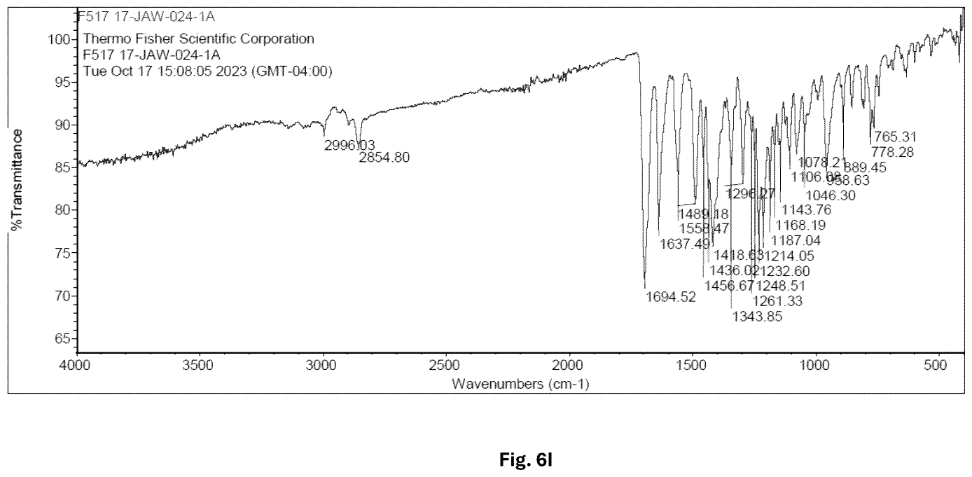

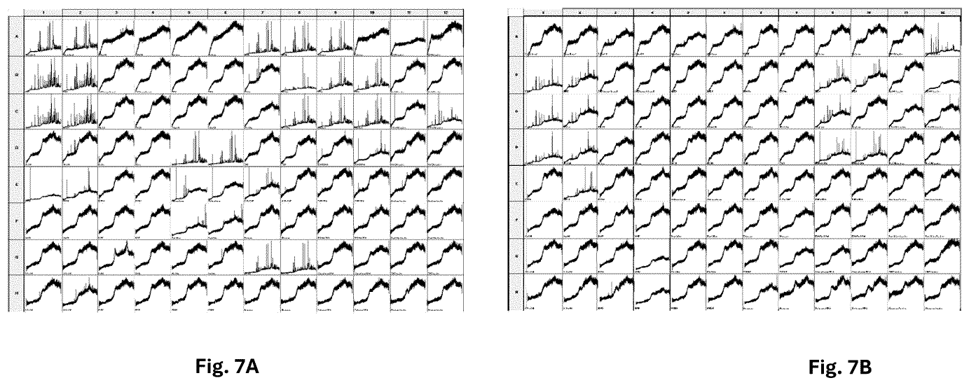

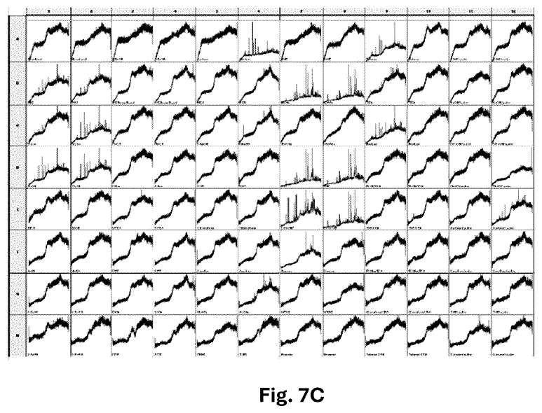

Approximately 10 mg of Compound 1 solid was added to each well on the slurry crystallizer as a heptane slurry. The solvents in each well were left to evaporate while the plates were covered with a single ply wipe.
Solvent mixtures were premixed before addition to solids.
Varied solvents (0.6 mL) were added to the slurry crystallizer (solvent placement shown in Table 11).
The slurry crystallizer was placed on Unchained Junior robot. Stirring was maintained at 250 rpm, and the chiller was set to hold at 45° C. overnight.
Antisolvents (0.3 mL) were manually added to the antisolvent crystallizer (antisolvent placement shown in Table 12).
The cooling plate was assembled.
After approximately 21 hours of stirring, filtration of 0.5 mL/well from the slurry crystallizer began. 0.5 mL from each well on the slurry crystallizer went through a hot filtration, and filtrate was divided between cooling (0.3 mL) and anti-solvent (0.2 mL) crystallizers.
Both cooling and antisolvent plates were set to cool to 0° C. (rate=1.0°/min) and held at 0° C. overnight.
The slurry crystallizer was opened after filtration was complete. All wells were observed to have the expected solvent level (~0.1 mL, or dry where no solvent was added). Stir bars were removed, and paper combs were inserted to wick solvents out of the crystallizer.
No abnormality was noticed in the disassembled filtrate plate.
After a total of 20 hours at 0° C., the cooling and antisolvent plates were brought back up to ambient temperature.
Both plates were unsealed, inspected, had stir bars removed, and had combs inserted to wick solvents out of crystallizers.
HTS-XRPD spectra were measured for each sample (FIGS. 7A-7C).

### Polymorph Screening Results

A range of solvents were employed to investigate the solubility of Compound 1 (Table 11 and Table 12). Solvents with different functional groups and polarity were selected. Solvent mixtures with water were explored (where miscible), to investigate the possibility of hydrate formation. Also, IPA-water mixtures were studied since the solubility of Compound 1 is known to be high in pure IPA. The material used in the solubility screening was enantiomerically pure. Heptane was used to transfer the solids to the slurry 96-well plate. This allowed for a suspension to be easily pipetted in a replicable manner. Heptane was selected as transferring solvent since it was found to have no impact on the crystallinity of Compound 1 (in amorphous form) after its removal.

TABLE 11
Polymorph Screening Solvents (placed in slurry crystallizer)

| | 1 | 2 | 3 | 4 | 5 | 6 | 7 | 8 | 9 | 10 | 11 | 12 |
|---|---|---|---|---|---|---|---|---|---|---|---|---|
| A | No | No | 2-BuOH | 2-BuOH | Acetone | Acetone | DME | DME | Toluene | Toluene | EtOH/ | EtOH/ |
| | solvent | solvent | | | | | | | | | water | water |
| B | PBS | PBS | 2- | 2- | MEK | MEK | EtOAc | EtOAc | TEA | TEA | AcOH/water | AcOH/water |
| | | | Ethoxy- | Ethoxy- | | | | | | | | |
| | | | ethanol | ethanol | | | | | | | | |
| C | Water | Water | MeCN | MeCN | t-AmOH | t-AmOH | iPrOAc | iPrOAc | Heptane | Heptane | 2-PrOH/ | 2-PrOH/ |
| | | | | | | | | | | | water | water |
| D | MeOH | MeOH | Nitro | Nitro | DCM | DCM | THF | THF | EtOH/IPA | EtOH/IPA | MeCN/water | MeCN/water |
| E | EtOH | EtOH | DMSO | DMSO | Chloro- | Chloro- | 2- | 2- | THF/IPA | THF/IPA | Acetone/ | Acetone/ |
| | | | | | form | form | MeTHF | MeTHF | | | water | water |
| F | AcOH | AcOH | DMF | DMF | Pyridine | Pyridine | Dioxane | Dioxane | EtOAc/ | EtOAc/ | Pyridine/ | Pyridine/ |
| | | | | | | | | | IPA | IPA | water | water |
| G | 1-PrOH | 1-PrOH | DMA | DMA | MeOAc | MeOAc | MTBE | MTBE | Chloro- | Chloro- | THF/water | THF/water |
| | | | | | | | | | form/IPA | form/IPA | | |
| H | 2-PrOH | 2-PrOH | NMP | NMP | MIBK | MIBK | Benzene | Benzene | Toluene/ | Toluene/ | Dioxane/ | Dioxane/ |
| | | | | | | | | | IPA | IPA | water | water |

Water mixtures: 10% water v/v; 2-Propanol mixtures: 10% IPA v/v

TABLE 12
Polymorph screening antisolvents (placed in antisolvent crystallizer)

| | 1 | 2 | 3 | 4 | 5 | 6 | 7 | 8 | 9 | 10 | 11 | 12 |
|---|---|---|---|---|---|---|---|---|---|---|---|---|
| A | blank | blank | Water | MTBE | MTBE | Heptane | Heptane | Heptane | Heptane | Heptane | Water | Water |
| B | Water | Water | Water | MTBE | MTBE | Heptane | Heptane | Heptane | Heptane | Heptane | Water | Water |
| C | Water | Water | Water | MTBE | MTBE | Heptane | Heptane | Heptane | Heptane | Heptane | Water | Water |
| D | Water | Water | Water | MTBE | MTBE | Heptane | Heptane | Heptane | Water | Water | Water | Water |
| E | Water | MTBE | Water | MTBE | MTBE | Heptane | Heptane | Heptane | MTBE | MTBE | Water | Water |
| F | Water | MTBE | Water | MTBE | MTBE | Heptane | Heptane | Heptane | MTBE | MTBE | MTBE | MTBE |
| G | Water | MTBE | Water | MTBE | MTBE | Heptane | Heptane | Heptane | MTBE | MTBE | MTBE | MTBE |
| H | Water | MTBE | Water | MTBE | MTBE | Heptane | Heptane | Heptane | MTBE | MTBE | MTBE | MTBE |

Based on the amount of remained solids in the slurry plate (visual assessment), the solubility of Compound 1 in different solvents was qualitatively categorized into low (<5 mg/mL), medium (5-15 mg/mL) and high (15<mg/ml). The amount of solids noted in the cooling and antisolvent plates were also used to revalidate the observation made from the slurry plate.

Conclusions: at the completion of the polymorph screening, 69 individual non-amorphous solids were isolated on 3 separate plates. XRPD was used to determine the polymorphic form of the solids. In total 10 different crystalline solid forms of Compound 1 were identified. Form C (see Example 6) was the dominating polymorph in this study followed by Form A (see Example 5) and Form J.

Forms A and B of Compound 1 were identified during the development study. In the slurry plate, several solvents resulted in formation of Form C. Amongst many other solvents, heptane was found to result in a mixture of Forms A and C, possibly due to slower conversion rate in this solvent. Form B was not observed in any of the wells. MeOH and mixture of IPA/water resulted in formation of Form J with a XRPD pattern mimicking Form A except for few missing peaks. Using pyridine as an organic base resulted in observation of Form D in the slurry plate.

Analyzing the cooling plate indicated the slow cooling method of recrystallization allows for harvesting more of Form J. In the cooling crystallization experiments done in this study, the samples were cooled to 0° C. with a rate of 1°/min and held at 0° C. overnight while solid was agitated using a stirrer bar. Only EtOH and the EtOH/IPA mixture resulted in Form C, which was observed under several conditions during the slurry conversions done at higher temperatures (45° C.). In addition, cooling from multiple solvents afforded Form G as the recrystallized form. Interestingly, no form A was detected in the cooling plate.

Several solvent systems yielded either Form J or C in the antisolvent experiment. Also, no form A was detected when antisolvent recrystallization was approached as was seen for the cooling plate. Very similar XRPD patterns observed for Forms A and J may suggest that Form A could be a mixture of Form J with other polymorph impurities. Also, new solid forms, namely Form E, F and I, were observed on the anti-solvent plate in addition to the solid forms that were discovered in the cooling plate.

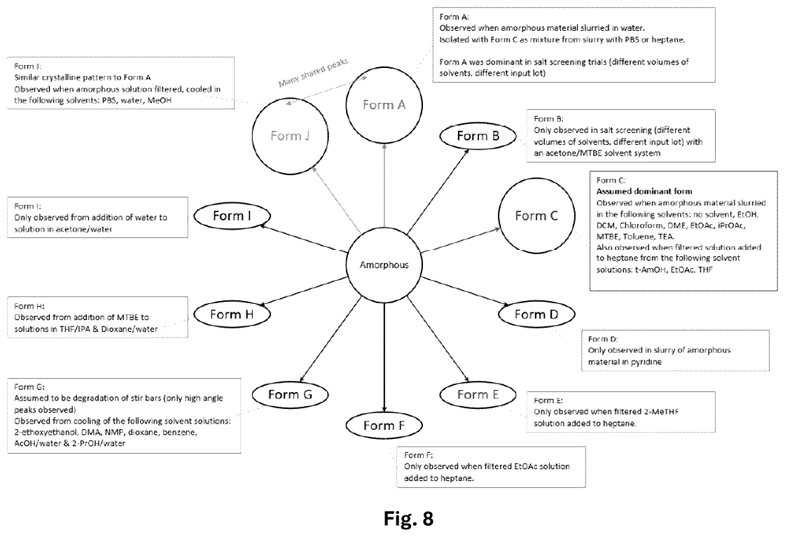

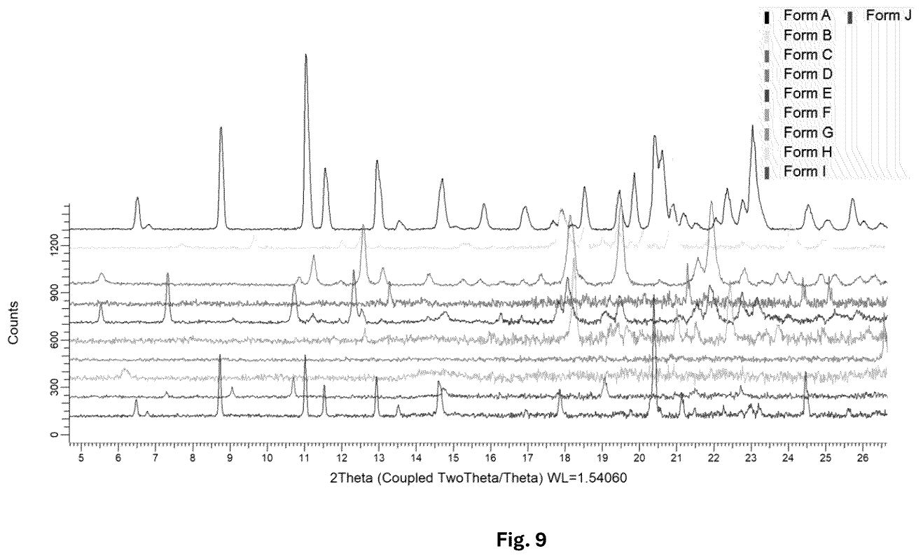

Conclusions: nine different crystalline solid forms of Compound 1 were identified in this HTS study to establish the polymorph landscape (FIG. 8). A comparison of XRPD diffractograms is provided in FIG. 9. The input material for this HTS was amorphous. The dominant forms observed in the screening were Forms A & J (closely related by XRPD, observed in 18 out of 264 experiments) and Form C (observed in 37 out of 264 experiments). Other forms (A, D, E, F, G, H, I) were found to appear less commonly (observed in a total of 21 out of 264 experiments). Amorphous forms were observed in 150 out of 264 experiments.

### Example 1: Synthesis and Characterization of Crystalline Compound 1 Form I

Crystalline Compound 1 Form I was prepared on 100 mg and 1.5 g scale according to the following procedure.

Amorphous Compound 1 (1.5 g) and a magnetic stir bar were charged into a 1-necked flask. To a separate vial, oxalic acid (347 mg) and IPA (20 V) were mixed at room temperature, producing a clear solution. The clear solution was added directly to the amorphous Compound 1 and stirred (500 rpm) at room temperature quickly dissolving the solids, resulting in a clear solution. The mixture was then heated to 70° C. and stirred at that temperature for 1 h. The resulting opaque solution was then cooled to 25° C., producing an immobile slurry. To this was added MTBE (20 V) making the slurry mobile. The suspension was then stirred overnight. The product was isolated by vacuum filtration and washed with an additional portion of MTBE (20 V). The solid was dried under vacuum with nitrogen counterbalance (yield: 1.42 g). ¹NMR spectrum in DMSO-d⁶ of Compound 1 oxalic acid is provided in FIG. 1L.

### Characterization

Oxalic acid content of Compound 1 Form I was determined by ion chromatography (IC) using an ion chromatography system with a suppressed conductivity detector. The sample was prepared 6 times and averaged. An oxalic acid level of 14.7% w/w was determined as free oxalic acid with respect to sodium oxalate standard. The theoretical oxalic acid content is 15.5% w/w as free oxalic acid, and extrapolated content of 15.9% w/w as oxalic acid. 14.7% w/w of oxalic acid represents an extrapolated value of 15.0% of oxalic acid in the crystalline form, which is consistent with one mole of oxalic acid for each mole of Compound 1. A representative chromatogram is provided in FIG. 1M.

The processed experimental x-ray diffraction pattern can be seen in FIG. 1A. The peak positions and relative intensities were identified in Table 1.

The processed DSC thermogram can be seen in FIG. 1B. The DSC thermogram shows an initial sharp endothermic peak that onsets at a temperature of 166° C., corresponding to the melting point of the sample. This is followed by two endothermic peaks with onset temperatures of 180° C., and 245° C., corresponding to the volatilization of the oxalic acid, and sample decomposition, respectively.

The processed TGA thermogram can be seen in FIG. 1C. The TGA thermogram shows two major mass loss events, occurring with onset temperatures of 179° C., and 241° C. These correspond to a two-step decomposition event, in which the oxalic acid volatilizes, followed by freebase decomposition. No solvent loss was observed by DSC or TGA.

### DVS Sample Preparation and Experimental Parameters:

6.691 mg of solid sample was loaded on a quartz pan and placed in the instrument for analysis.

A matched, empty reference pan was placed in an adjacent chamber and exposed to identical conditions. Two cycles of sorption and desorption were carried out at 25° C. (0 to 90% RH). Each step in RH was held until the derivative of mass with respect to time reached 0.0075%/min or less. The maximum duration at each RH was 300 minutes.

### DVS Results:

The change in mass over time (FIG. 1D, left panel), isotherm plot (FIG. 1D, right panel), and hysteresis values (Table 13) show that the sample absorbed moisture. It can be seen that rapid uptake of moisture began at around 50% RH. The isotherm plot shows the reversibility of the moisture uptake. After the 1st cycle, the moisture uptake reached 1.42% by mass relative to the initial sample mass at about 90% RH, indicating the material is slightly hygroscopic. Upon the 1st desorption, the sample retained 0.48% wt, with the moisture loss occurring gradually between 0-90% RH. During the 2nd cycle, the sample reabsorbs a near identical amount of water, with a more gradual uptake of water absorbed over a wide RH range (0-90%). An XRPD diffractogram was measured post-DVS analysis FIG. 1E). The resulting XRPD patterns suggest the material retains its form upon humidity exposure.

TABLE 13
DVS Hysteresis Data for Crystalline Compound 1 Form I

| | Target Relative | Change in mass (%) | | |
|---|---|---|---|---|
| Cycle number | Humidity (%) | Sorption | Desorption | Hysteresis |
| 1 | 0.0 | 0.000 | 0.480 | |
| 1 | 10.0 | 0.118 | 0.632 | 0.514 |
| 1 | 20.0 | 0.161 | 0.691 | 0.529 |
| 1 | 30.0 | 0.198 | 0.745 | 0.547 |
| 1 | 40.0 | 0.193 | 0.801 | 0.608 |
| 1 | 50.0 | 0.172 | 0.863 | 0.691 |
| 1 | 60.0 | 0.329 | 0.933 | 0.604 |
| 1 | 70.0 | 0.676 | 1.024 | 0.348 |
| 1 | 80.0 | 1.064 | 1.163 | 0.099 |
| 1 | 90.0 | 1.420 | 1.420 | |
| 2 | 0.0 | 0.480 | 0.395 | |
| 2 | 10.0 | 0.596 | 0.550 | −0.046 |
| 2 | 20.0 | 0.646 | 0.679 | 0.033 |
| 2 | 30.0 | 0.701 | 0.729 | 0.028 |
| 2 | 40.0 | 0.758 | 0.785 | 0.027 |
| 2 | 50.0 | 0.819 | 0.844 | 0.025 |
| 2 | 60.0 | 0.888 | 0.913 | 0.025 |
| 2 | 70.0 | 0.982 | 1.001 | 0.019 |
| 2 | 80.0 | 1.125 | 1.136 | 0.010 |
| 2 | 90.0 | 1.390 | 1.390 | |

PLM images for the 100 mg batch synthesis of the crystalline Compound 1 Form I is provided in FIG. 1F at 4× magnification (left panel) and 40× magnification (right panel). PLM images for the 1.5 g batch synthesis of the Compound 1 Form I is provided in FIG. 1G at 4× magnification (left panel) and 40× magnification (right panel).

Scanning Electron Microscopy (SEM) provides information about the shape and size of the particles in a sample. When using the backscatter detector for the imaging, brighter regions correlate to areas where the electron density is greater, i.e., heavier elements. SEM Energy Dispersive X-ray Spectroscopy (EDS) can be used in the SEM to determine the elemental composition of a region.

A SEM image of the sample can be seen in FIG. 1H. The Compound 1 oxalic acid is composed of small needles that aggregate to form large clumps. No additional elements were observed, suggesting that no inorganic impurities were present within the sample.

The FTIR spectrum can be seen in FIG. 1I. The measured spectrum meets expectations of the crystalline Compound 1 Form I. An exceptionally broad peak between 3250-2400 cm⁻¹ can be assigned to the carboxylic O—H stretch corresponding to the oxalic acid present within the sample. A series of peaks between 3065-2885 cm⁻¹ can be assigned to alkane and alkene C—H stretches within the freebase structure. Additionally, sharp peaks centered on 1694 and 1627 cm⁻¹ can be assigned to the cyclic and non-cyclic C═O amide stretches within the freebase structure. An additional peak centered on 1781 cm⁻¹ can be assigned to the carboxylic C═O stretch corresponding to oxalic acid.

Single crystals of the Compound 1 Form I were grown by dissolving the solid compound in 2-propanol. The solution was filtered and slowly cooled with no agitation. The resulting large crystals were tested to determine the crystal structure (FIG. 1J). Single crystal x-ray diffraction (SCXRD) confirms that there is one oxalic acid and one free base in each unit cell of the crystal structure. Estimated bond lengths from the H of O5 to O4 (1.496 Å) and H of O6 to N2 (1.667 Å) are within the typical hydrogen bond length (~1.5 to 2.5 Å) (FIG. 1K).

### Example 2: Synthesis and Characterization of Crystalline Compound 1 Form II

Crystalline Compound 1 Form II was prepared on 100 mg and 300 mg scale according to the following procedure.

Amorphous Compound 1 (300 mg) and a magnetic stir bar were charged into a 1-necked flask. To a separate vial, hydrobromic acid (85.5 μL) and IPA (20 V) were mixed at room temperature, producing a clear solution. The clear solution was added directly to the amorphous Compound 1 and stirred (500 rpm) at room temperature. The clear mixture was then heated to 70° C. and stirred at that temperature for 1 h. Within 40 minutes the solution became opaque. The suspension was then cooled to 25° C., producing a mobile slurry. To this was added MTBE (20 V). The suspension was then stirred overnight. The product was isolated by vacuum filtration and washed with an additional portion of MTBE (20 V). The solid was dried under vacuum with nitrogen counterbalance (uncorrected yield: 323.7 mg).

### Characterization

The processed experimental x-ray diffraction pattern can be seen in FIG. 2A. The peak positions and relative intensities were identified in Table 2.

The processed DSC thermogram can be seen in FIG. 2B. The DSC thermogram shows an exceptionally broad endothermic event that onsets at a temperature of 53° C., centered on 89° C., corresponding to loss of reaction solvent and surface moisture. This is followed by two subsequent thermal events that are partially overlapping. The first event is a broad endothermic peak that onsets at a temperature of 219° C., followed by a sharp exothermic peak that onsets at a temperature of 234° C. The initial endothermic peak corresponds to the melting point of the sample, followed immediately by an exothermic event.

The processed TGA thermogram can be seen in FIG. 2C. The TGA thermogram shows a gradual mass loss of 3.6% between 25 and 235° C., corresponding to a loss of residual reaction solvent (IPA and MTBE) and surface moisture. As the temperature increases, a secondary mass loss is observed that onsets at a temperature of 235° C., corresponding to sample decomposition.

### DVS Sample Preparation and Experimental Parameters:

11.73 mg of solid sample was loaded on a quartz pan and placed in the instrument for analysis.

A matched, empty reference pan was placed in an adjacent chamber and exposed to identical conditions. Two cycles of sorption and desorption were carried out at 25° C. (0 to 90% RH). Each step in RH was held until the derivative of mass with respect to time reached 0.0075%/min or less. The maximum duration at each RH was 300 minutes.

### DVS Results:

The change in mass over time (FIG. 2D, left panel), isotherm plot (FIG. 2D, right panel), and hysteresis values (Table 14) show that the sample absorbed moisture. It can be seen that a steep uptake of moisture began at around 60% RH. The isotherm plot shows the reversibility of the moisture uptake. After the 1st cycle, the moisture uptake reached up to 11.68 by mass relative to the initial sample mass at about 90% RH, indicating the material is moderately hygroscopic. Upon the 1st desorption, the sample retained 2.82% wt, with most of the moisture loss occurring between 0-10% RH. During the 2nd cycle, the sample reabsorbs a near identical amount of water, this time the majority of the water is absorbed at a much lower RH (10% vs. 60%). An XRPD diffractogram was measured post-DVS analysis (FIG. 2F). The resulting diffractograms suggest a form change upon hydration.

TABLE 14
DVS Hysteresis Data for Crystalline Compound 1 Form II

| | Target Relative | Change in mass (%) | | |
|---|---|---|---|---|
| Cycle number | Humidity (%) | Sorption | Desorption | Hysteresis |
| 1 | 0.0 | 0.00 | 2.82 | |
| 1 | 10.0 | 1.63 | 7.96 | 6.34 |
| 1 | 20.0 | 2.03 | 8.61 | 6.58 |
| 1 | 30.0 | 2.32 | 9.05 | 6.73 |
| 1 | 40.0 | 2.59 | 9.38 | 6.79 |
| 1 | 50.0 | 2.94 | 9.69 | 6.75 |
| 1 | 60.0 | 3.41 | 10.02 | 6.61 |
| 1 | 70.0 | 4.86 | 10.40 | 5.54 |
| 1 | 80.0 | 10.88 | 10.88 | 0.01 |
| 1 | 90.0 | 11.68 | 11.68 | |
| 2 | 0.0 | 2.82 | 2.90 | |
| 2 | 10.0 | 7.47 | 7.94 | 0.47 |
| 2 | 20.0 | 8.22 | 8.54 | 0.32 |
| 2 | 30.0 | 8.69 | 8.95 | 0.26 |
| 2 | 40.0 | 9.08 | 9.29 | 0.21 |
| 2 | 50.0 | 9.46 | 9.58 | 0.11 |
| 2 | 60.0 | 9.84 | 9.89 | 0.05 |
| 2 | 70.0 | 10.23 | 10.24 | 0.02 |
| 2 | 80.0 | 10.70 | 10.69 | −0.01 |
| 2 | 90.0 | 11.40 | 11.40 | |

PLM images for the 100 mg batch synthesis of the crystalline Compound 1 Form 11 is provided in FIG. 2F at 4× magnification (left panel) and 40× magnification (right panel). PLM images for the 300 mg batch synthesis of the Compound 1 Form 11 is provided in FIG. 2G at 4× magnification (left panel) and 40× magnification (right panel).

A SEM Image of the sample can be seen in FIG. 2H. The Compound 1 Form II is composed of small particles that aggregate to form large clumps. No additional elements were observed, suggesting that no inorganic impurities were present within the sample.

The FTIR spectrum can be seen in FIG. 2I. The measured spectrum meets expectations of the Compound 1 Form II. A broad peak centered on 3437 cm⁻¹ can be assigned to the O—H stretch corresponding to residual IPA solvent present within the sample. A series of peaks between 3122-2885 cm⁻¹ can be assigned to alkane and alkene C—H stretches within the freebase structure, as well as residual MTBE solvent present. Additionally, sharp peaks centered on 1681 and 1634 cm⁻¹ can be assigned to the cyclic and non-cyclic C═O amide stretches within the freebase structure.

### Example 3: Synthesis and Characterization of Crystalline Compound 1 Form III

Crystalline Compound 1 Form III was prepared on 50 mg and 300 mg scale according to the following procedure.

300 mg Scale: Amorphous Compound 1 (300 mg) and a magnetic stir bar were charged into a 20 mL vial. To a separate vial, hydrochloric acid (10.3 μL) and IPA (20 V) were mixed at room temperature, producing a clear solution. The clear solution was added directly to the amorphous Compound 1 and stirred (500 rpm) at room temperature. Solids remained undissolved on the sides of the vial. The mixture was then heated to 70° C., fully dissolving the remaining solids, and stirred at that temperature for 1 h. The solution was then cooled to 25° C., yielding a clear solution. To this was added MTBE (20 V) producing an opaque solution that was stirred overnight. The product was isolated by vacuum filtration and then washed with additional MTBE (20 V). The resulting solid was dried under vacuum with nitrogen counterbalance (yield: 263.6 mg).

### Characterization

The processed experimental x-ray diffraction pattern can be seen in FIG. 3A. The peak positions and relative intensities were identified in Table 3.

The processed DSC thermogram can be seen in FIG. 3B. The DSC thermogram shows an initial broad endothermic peak that onsets at a temperature of 42° C., corresponding to the loss of reaction solvent (IPA and MTBE) and surface moisture. This is followed by a small, noisy endothermic peak centered on 131° C. Due to the lack of a mass loss event at this temperature in the TGA thermogram, this is assigned as the melting point of the sample. Finally, a major endothermic peak is observed that onsets at a temperature of 192° C., corresponding to the volatilization of the HCl, and decomposition of the sample.

The processed TGA thermogram can be seen in FIG. 3C. The TGA thermogram shows a gradual mass loss of 1.7% between 25 and 84° C., corresponding to a loss of reaction solvent (IPA and MTBE) and surface moisture. As the temperature increases, two additional mass loss events are observed centered on 187° C., and 249° C. This corresponds to a two-step decomposition event, in which the hydrochloric acid volatilizes followed by freebase decomposition.

### DVS Sample Preparation and Experimental Parameters:

3.628 mg of solid sample was loaded on a quartz pan and placed in the instrument for analysis. A matched, empty reference pan was placed in an adjacent chamber and exposed to identical conditions. Two cycles of sorption and desorption were carried out at 25° C. (0 to 90% RH). Each step in RH was held until the derivative of mass with respect to time reached 0.0013%/min or less. The maximum duration at each RH was 300 minutes.

### DVS Results:

The change in mass over time (FIG. 3D, left panel), isotherm plot (FIG. 3D, right panel), and hysteresis values (Table 15) show that the sample absorbed moisture. It can be seen that a steep uptake of moisture began at around 60% RH. The isotherm plot shows the reversibility of the moisture uptake. After the 1st cycle, the moisture uptake reached 19.18% by mass relative to the initial sample mass at about 90% RH, indicating the material is hygroscopic. Upon the 1st desorption, the sample retained 4.68% wt, with most of the moisture loss occurring between 0-10% RH. During the 2nd cycle, the sample reabsorbs a near identical amount of water, this time most of the water is absorbed at a much lower RH (10% vs. 60%). An XRPD diffractogram was measured post-DVS analysis (FIG. 3E). The resulting pattern change suggest a form change upon hydration.

TABLE 15
DVS Hysteresis Data for Compound 1 Form III

| | Target Relative | Change in mass (%) | | |
|---|---|---|---|---|
| Cycle number | Humidity (%) | Sorption | Desorption | Hysteresis |
| 1 | 0.0 | 0.00 | 4.68 | |
| 1 | 10.0 | 0.40 | 13.79 | 13.39 |
| 1 | 20.0 | 0.77 | 14.79 | 14.02 |
| 1 | 30.0 | 2.75 | 15.34 | 12.59 |
| 1 | 40.0 | 4.80 | 15.77 | 10.97 |
| 1 | 50.0 | 5.52 | 16.18 | 10.66 |
| 1 | 60.0 | 6.49 | 16.64 | 10.14 |
| 1 | 70.0 | 18.03 | 17.17 | −0.86 |
| 1 | 80.0 | 19.61 | 17.88 | −1.74 |
| 1 | 90.0 | 19.18 | 19.18 | |
| 2 | 0.0 | 4.68 | 4.19 | |
| 2 | 10.0 | 10.35 | 13.38 | 3.02 |
| 2 | 20.0 | 11.85 | 14.15 | 2.31 |
| 2 | 30.0 | 13.15 | 14.67 | 1.52 |
| 2 | 40.0 | 14.42 | 15.12 | 0.69 |
| 2 | 50.0 | 15.06 | 15.53 | 0.47 |
| 2 | 60.0 | 15.79 | 16.00 | 0.21 |
| 2 | 70.0 | 16.57 | 16.58 | 0.02 |
| 2 | 80.0 | 17.32 | 17.34 | 0.02 |
| 2 | 90.0 | 18.86 | 18.86 | |

PLM images for the 100 mg batch synthesis of the crystalline Compound 1 Form III is provided in FIG. 3F at 4× magnification (left panel) and 40× magnification (right panel). PLM images for the 300 mg batch synthesis of the Compound 1 Form III is provided in FIG. 3G at 4× magnification (left panel) and 40× magnification (right panel).

A SEM image of the sample can be seen in FIG. 3H. The crystalline Compound 1 Form III is composed of small particles that aggregate to form large clumps. No additional elements were observed, suggesting that no inorganic impurities were present within the sample.

The FTIR spectrum can be seen in FIG. 3I. The measured spectrum meets expectations of the crystalline Compound 1 Form III. A broad peak centered on 3363 cm⁻¹ can be assigned to the O—H stretch corresponding to residual IPA solvent present within the sample. A series of unlabeled peaks centered around 3000 cm⁻¹ can be assigned to C—H stretches within the freebase structure and residual MTBE solvent. Sharp peaks centered on 1665 and 1633 cm⁻¹ can be assigned to the cyclic and non-cyclic C═O amide stretches within the freebase structure.

### Example 4: Synthesis and Characterization of Crystalline Compound 1 Form IV

Crystalline Compound 1 Form IV was prepared on 100 mg and 1.5 g scale according to the following procedure.

Compound 1 (1.5 g) and a magnetic stir bar were charged into a 1-necked flask. To a separate vial, phosphoric acid (338 μL) and IPA (20 V) were mixed at room temperature, producing a clear solution. The clear solution was added directly to the amorphous Compound 1 and stirred (500 rpm) at room temperature. Solids stick to the sides of the flask. The mixture was then heated to 70° C. As the temperature surpassed 60° C., the solution became clear. The solution was stirred at 70° C. for 1 h and remained clear. The solution was then cooled to 25° C. and remained clear. To this was added MTBE (20 V). The solution remained clear during the addition but became progressively opaquer upon continual stirring overnight. After stirring overnight, the solution became immobile. The product was isolated by vacuum filtration and transferred with an additional portion of MTBE (20 V) that acted as both a wash and to improve the mobility of the suspension for ease of transfer. The solid was dried under vacuum with nitrogen counterbalance (yield: 1.49 g).

### Characterization

The processed experimental x-ray diffraction pattern can be seen in FIG. 4A. The peak positions and relative intensities were identified in Table 4.

The processed DSC thermogram can be seen in FIG. 4B. The DSC thermogram shows a small, and large, broad endothermic peaks with onset temperatures of 40° C., and 78° C., respectively. These correspond to reaction solvent loss. This is followed by an endothermic peak that onsets at a temperature of 154° C., corresponding to the melting of the sample. Finally, a fourth endothermic peak is observed that onsets at a temperature of 221° C., corresponding to the decomposition of the sample.

The processed TGA thermogram can be seen in FIG. 4C. The TGA thermogram shows an initial mass loss of 5.72% that onsets at a temperature of 73° C., corresponding to a loss of residual reaction solvent (IPA and MTBE) and surface moisture. As the temperature increased, a secondary mass loss was observed that onsets at a temperature of 194° C., corresponding to sample decomposition.

### DVS Sample Preparation and Experimental Parameters:

12.27 mg of solid sample was loaded on a quartz pan and placed in the instrument for analysis.

A matched, empty reference pan was placed in an adjacent chamber and exposed to identical conditions. Two cycles of sorption and desorption were carried out at 25° C. (0 to 90% RH). Each step in RH was held until the derivative of mass with respect to time reached 0.0038%/min or less.

The maximum duration at each RH was 300 minutes.

### DVS Results:

The change in mass over time (FIG. 4D, left panel), isotherm plot (FIG. 4D, right panel), and hysteresis (Table 16) values show that the sample absorbed moisture. It can be seen that a steep uptake of moisture began at around 0% RH, and then continued gradually until 90% RH. The isotherm plot shows the reversibility of the moisture uptake. After the 1st cycle, the moisture uptake reached 7.18% by mass relative to the initial sample mass at about 90% RH, indicating the material is moderately hygroscopic. Upon the 1st desorption, the sample retained 0.25% wt, with the moisture loss occurring gradually between 0-90% RH. During the 2nd cycle, the sample reabsorbs a near identical amount of water. An XRPD diffractogram was measured post-DVS analysis (FIG. 4E). The resulting XRPD patterns suggest the material retains its form upon humidity exposure.

TABLE 16
DVS Hysteresis Data for Compound 1 Form IV

| | Target Relative | Change in mass (%) | | |
|---|---|---|---|---|
| Cycle number | Humidity (%) | Sorption | Desorption | Hysteresis |
| 1 | 0.0 | 0.000 | 0.245 | |
| 1 | 10.0 | 2.398 | 3.723 | 1.325 |
| 1 | 20.0 | 3.324 | 4.413 | 1.089 |
| 1 | 30.0 | 3.995 | 4.888 | 0.893 |
| 1 | 40.0 | 4.576 | 5.379 | 0.803 |
| 1 | 50.0 | 5.133 | 5.691 | 0.558 |
| 1 | 60.0 | 5.626 | 5.947 | 0.321 |
| 1 | 70.0 | 6.054 | 6.224 | 0.170 |
| 1 | 80.0 | 6.496 | 6.572 | 0.076 |
| 1 | 90.0 | 7.182 | 7.182 | |
| 2 | 0.0 | 0.245 | 0.258 | |
| 2 | 10.0 | 2.554 | 3.708 | 1.154 |
| 2 | 20.0 | 3.460 | 4.402 | 0.942 |
| 2 | 30.0 | 4.106 | 4.878 | 0.772 |
| 2 | 40.0 | 4.618 | 5.334 | 0.716 |
| 2 | 50.0 | 5.107 | 5.654 | 0.547 |
| 2 | 60.0 | 5.615 | 5.915 | 0.300 |
| 2 | 70.0 | 6.042 | 6.190 | 0.148 |
| 2 | 80.0 | 6.470 | 6.537 | 0.067 |
| 2 | 90.0 | 7.126 | 7.126 | |

PLM images for the 100 mg batch synthesis of the Compound 1 Form IV is provided in FIG. 4F at 4× magnification (left panel) and 40× magnification (right panel). PLM images for the 1.5 g batch synthesis of the Compound 1 Form IV is provided in FIG. 4G at 4× magnification (left panel) and 40× magnification (right panel).

A SEM image of the sample can be seen in FIG. 4H. The crystalline Compound 1 Form IV is composed of small particles that aggregate to form large clumps. No additional elements were observed, suggesting that no inorganic impurities were present within the sample.

The FTIR spectrum can be seen in FIG. 4I. The measured spectrum meets expectations of the crystalline Compound 1 Form IV. An exceptionally broad peak between 3750-2000 cm⁻¹ can be assigned to the multitude of O—H stretches corresponding to both the phosphoric acid, and the residual IPA solvent present within the sample. Additionally, sharp peaks centered on 1674 and 1633 cm⁻¹ can be assigned to the cyclic and non-cyclic C═O amide stretches within the freebase structure. An additional peak centered on 1698 cm⁻¹ can be assigned to the P—O stretch corresponding to phosphoric acid.

### Example 5: Synthesis and Characterization of Compound 1 Form A Freebase

Compound 1 Form A freebase was prepared on 300 mg scale according to the following procedure. [0222]300 mg Scale: Amorphous Compound 1 (300 mg) and a magnetic stir bar were charged into a 20 mL vial and stirred (250 rpm). The vial was heated to 50° C., and portion wise methanol (MeOH, 7 V) was added until complete compound dissolution. The solution was cooled from 50° C. to 0° C. at a rate of 0.5° C./min and stirred overnight at 0° C. A white solid was collected by vacuum filtration, and then dried under vacuum with nitrogen counterbalance (yield: 260.7 mg).

### Characterization

The processed experimental x-ray diffraction pattern can be seen in FIG. 5A. The peak positions and relative intensities were identified in Table 5.

The processed DSC thermogram can be seen in FIG. 5B. The DSC thermogram shows an initial broad endothermic peak that onsets at a temperature of 80° C., likely corresponding to the same loss of residual reaction solvent (MeOH), as noted previously in the TGA thermogram. As the temperature increases, a second endothermic peak is observed that onsets at a temperature of 146° C. This sharp peak corresponds to the melting of Form A. Finally, a third broad endothermic peak is observed that onsets at a temperature of 181° C., corresponding to the decomposition of the sample.

The processed TGA thermogram can be seen in FIG. 5C. The TGA thermogram shows an initial mass loss of 5.69% that onsets at a temperature of 56° C., likely corresponding to a loss of residual reaction solvent (MeOH). As the temperature increases, a secondary mass loss is observed that onsets at a temperature of 216° C., corresponding to sample decomposition.

### DVS Sample Preparation and Experimental Parameters:

5.57 mg of solid sample was loaded on a quartz pan and placed in the instrument for analysis. A matched, empty reference pan was placed in an adjacent chamber and exposed to identical conditions. Two cycles of sorption and desorption were carried out at 25° C. (0 to 90% RH). Each step in RH was held until the derivative of mass with respect to time reached 0.009%/min or less. The maximum duration at each RH was 300 minutes.

### DVS Results:

The change in mass over time (FIG. 5D, left panel), isotherm plot (FIG. 5D, right panel), and hysteresis values (Table 17) show that the sample absorbed moisture. It can be seen that a steep uptake of moisture began at around 40% RH. The isotherm plot shows the irreversibility of the moisture uptake. After the 1st cycle, the final mass increased by approximately 6%. Upon the 2nd cycle, the amount adsorbed is consistent with the 1st cycle, and retains the same amount of water upon desorption. The hysteresis calculated in Table 17 is the percentage change in mass from the sorption to desorption in a single cycle. Large hysteresis at high relative humidity indicates the formation of hydrates. The moisture uptake reached up to 14.7% by mass relative to the initial sample mass (90% RH). This indicates that the sample is hygroscopic, which should be considered when packaging of the final product is considered. An XRPD diffractogram was measured post-DVS analysis. The resulting pattern change suggest a form change upon hydration.

TABLE 17
DVS Hysteresis Data for Compound 1 Form A Freebase

| | Target | | | |
|---|---|---|---|---|
| | Relative | | | |
| | Humidity | Change in mass (%) | | |
| Cycle number | (%) | Sorption | Desorption | Hysteresis |
| 1 | 0.0 | 0.00 | 5.87 | |
| 1 | 10.0 | 0.15 | 6.53 | 6.38 |
| 1 | 20.0 | 0.22 | 13.88 | 13.66 |
| 1 | 30.0 | 0.47 | 13.99 | 13.52 |
| 1 | 40.0 | 2.01 | 14.07 | 12.07 |
| 1 | 50.0 | 4.32 | 14.16 | 9.84 |
| 1 | 60.0 | 11.57 | 14.24 | 2.67 |
| 1 | 70.0 | 13.47 | 14.33 | 0.86 |
| 1 | 80.0 | 14.19 | 14.45 | 0.27 |
| 1 | 90.0 | 14.66 | 14.66 | |
| 2 | 0.0 | 5.87 | 5.89 | |
| 2 | 10.0 | 5.96 | 6.64 | 0.68 |
| 2 | 20.0 | 6.00 | 13.88 | 7.88 |
| 2 | 30.0 | 6.05 | 13.99 | 7.94 |
| 2 | 40.0 | 6.11 | 14.09 | 7.98 |
| 2 | 50.0 | 8.19 | 14.18 | 5.99 |
| 2 | 60.0 | 13.90 | 14.26 | 0.36 |
| 2 | 70.0 | 14.30 | 14.36 | 0.06 |
| 2 | 80.0 | 14.45 | 14.49 | 0.04 |
| 2 | 90.0 | 14.72 | 14.72 | |

PLM images for the 300 mg batch synthesis of the Compound 1 Form A freebase is provided in FIG. 5F at 4× magnification (left panel) and 40× magnification (right panel).

A SEM image of the sample can be seen in FIG. 5G. Freebase Form A is composed of small needles that aggregate to form large clumps. No additional elements were observed, suggesting that no inorganic impurities were present within the sample.

The FTIR spectrum can be seen in FIG. 5H. The measured spectrum meets expectations of freebase Form A. A broad peak centered on 3390 cm⁻¹ can be assigned to the O—H stretch corresponding to residual MeOH solvent present within the sample. A series of peaks between 2999-2932 cm⁻¹ can be assigned to alkane and alkene C—H stretches within the freebase structure. Additionally, sharp peaks centered on 1692 and 1673 cm⁻¹ can be assigned to the cyclic and non-cyclic C═O amide stretches within the freebase structure.

### Example 6: Synthesis and Characterization of Compound 1 Form C Freebase

Compound 1 Form C freebase was prepared on 80 mg and 500 mg scale according to the following procedure.

500 mg Scale: A mixture of crystalline forms of Compound 1 (503 mg) and isopropyl acetate (iPrOAc, 20 V) was charged into a 20 mL vial and heated to 50° C. with top stirring. No dissolution was observed over 30 minutes. The solution was cooled to room temperature and continued to stir for 20 hours. Solids were collected by vacuum filtration under a blanket of nitrogen, and then dried under vacuum with nitrogen counterbalance (yield: 430 mg).

### Characterization

The processed experimental x-ray diffraction pattern can be seen in FIG. 6A. The peak positions and relative intensities were identified in Table 6.

The processed DSC thermogram can be seen in FIG. 6B. The DSC thermogram shows a sharp endothermic step that onsets at a temperature of 167° C., corresponding to the melting point of the sample. This is followed by a broad endothermic peak that onsets at a temperature of 258° C., corresponding to the decomposition of the sample.

The processed TGA thermogram can be seen in FIG. 5C. The TGA thermogram shows a gradual mass loss of 0.6% between 25 and 266° C., corresponding to a loss of trace reaction solvent (EtOAc) and surface moisture. As the temperature increases, a secondary mass loss is observed that onsets at a temperature of 266° C., corresponding to sample decomposition.

### DVS Sample Preparation and Experimental Parameters:

7.30 mg of solid sample was loaded on a quartz pan and placed in the instrument for analysis. A matched, empty reference pan was placed in an adjacent chamber and exposed to identical conditions. Two cycles of sorption and desorption were carried out at 25° C. (0 to 90% RH). Each step in RH was held until the derivative of mass with respect to time reached 0.0026%/min or less. The maximum duration at each RH was 300 minutes.

### DVS Results:

The change in mass over time (FIG. 6D, left panel), isotherm plot (FIG. 6D, right panel), and hysteresis values (Table 18) show that the sample absorbed moisture. It can be seen that Form C uptakes a minimal amount of moisture that absorbs gradually from 0 to 90% RH. The isotherm plot shows the reversibility of the moisture uptake. After the 1st cycle, the final mass increased by approximately 0.148%. Upon the 2nd cycle, the amount adsorbed is consistent with the 1st cycle, and retains the same amount of water upon desorption. The hysteresis calculated in Table 18 is the percentage change in mass from the sorption to desorption in a single cycle. Form C exhibits minimal hysteresis. Form C is non-hygroscopic, which should be considered when packaging of the final product is considered. An XRPD diffractogram was measured post-DVS analysis (FIG. 6E). The resulting XRPD patterns suggest the material retains its form upon humidity exposure.

TABLE 18
DVS Hysteresis Data for Compound 1 Form C Freebase

| | Target Relative | Change in mass (%) | | |
|---|---|---|---|---|
| Cycle number | Humidity (%) | Sorption | Desorption | Hysteresis |
| 1 | 0.0 | 0.0000 | −0.0041 | |
| 1 | 10.0 | 0.0151 | 0.0123 | −0.0027 |
| 1 | 20.0 | 0.0274 | 0.0260 | −0.0014 |
| 1 | 30.0 | 0.0400 | 0.0384 | −0.0016 |
| 1 | 40.0 | 0.0521 | 0.0507 | −0.0014 |
| 1 | 50.0 | 0.0644 | 0.0671 | 0.0027 |
| 1 | 60.0 | 0.0795 | 0.0822 | 0.0027 |
| 1 | 70.0 | 0.0946 | 0.1000 | 0.0055 |
| 1 | 80.0 | 0.1137 | 0.1192 | 0.0055 |
| 1 | 90.0 | 0.1480 | 0.1480 | |
| 2 | 0.0 | −0.0041 | −0.0123 | |
| 2 | 10.0 | 0.0082 | 0.0041 | −0.0041 |
| 2 | 20.0 | 0.0219 | 0.0175 | −0.0044 |
| 2 | 30.0 | 0.0329 | 0.0288 | −0.0041 |
| 2 | 40.0 | 0.0425 | 0.0411 | −0.0014 |
| 2 | 50.0 | 0.0534 | 0.0548 | 0.0014 |
| 2 | 60.0 | 0.0644 | 0.0685 | 0.0041 |
| 2 | 70.0 | 0.0795 | 0.0866 | 0.0071 |
| 2 | 80.0 | 0.0987 | 0.1055 | 0.0069 |
| 2 | 90.0 | 0.1329 | 0.1329 | |

PLM images for the 80 mg batch synthesis of the Compound 1 Form C freebase is provided in FIG. 6F at 4× magnification (left panel) and 40× magnification (right panel). PLM images for the 500 mg batch synthesis of the Compound 1 Form C freebase is provided in FIG. 6G at 4× magnification (left panel) and 40× magnification (right panel).

A SEM image of the sample can be seen in FIG. 6H. Freebase Form C is composed of small plates. No additional elements were observed, suggesting that no inorganic impurities were present within the sample.

The FTIR spectrum can be seen in FIG. 6I. The measured spectrum meets expectations of freebase Form C. In contrast to Form A, in Form C there is the absence of a broad peak centered on 3390 cm¹. This is owing to the lack of residual solvent within the sample, which is supported by the absence of a mass loss below 150° C. in the TGA thermogram. A series of peaks between 2996-2855 cm⁻¹ can be assigned to alkane and alkene C—H stretches within the freebase structure. Additionally, sharp peaks centered on 1695 and 1637 cm⁻¹ can be assigned to the cyclic and non-cyclic C═O amide stretches within the freebase structure.

### Characterization Summary (Table 19)

Crystallization conditions for six Compound 1 solid forms were presented, and the resulting solids were characterized by XRPD, DSC, TGA, SEM-EDS, FTIR and DVS. Of the six forms, Compound 1 Form C freebase and Compound 1 Form I both exhibit sharp endothermic peaks corresponding to their associated melting points. Both exhibit minimal solvent loss in their TGA thermograms, uptake a small amount of water at high relative humidities by DVS, and do not undergo a form change upon humidity exposure.

TABLE 19
Summary Characterization Table of Crystalline Forms

| | | Freebase A | | Freebase C | | HBr | | HCl | | C₂H₂O₄ | | H₃PO₄ | |
|---|---|---|---|---|---|---|---|---|---|---|---|---|---|
| Crystallization | | | | | | | | | | | | | |
| Trial #1 | Scale | 300 | mg | 80 | mg | 100 | mg | 50 | mg | 100 | mg | 100 | mg |
| | Yield | 260.7 | mg | 52 | mg | 111.3 | mg | 53.3 | mg | 82.5 | mg | 73.6 | mg |

| | | | | | | | | | | | | |
|---|---|---|---|---|---|---|---|---|---|---|---|---|
| Trial #2 | Scale | NA | 500 | mg | 300 | mg | 300 | mg | 1.5 | g | 1.5 | g |
| | Yield | NA | 430 | mg | 323.7 | mg | 263.6 | mg | 1.42 | g | 1.49 | g |

| DSC/TGA | | | | | | | | | | |
|---|---|---|---|---|---|---|---|---|---|---|
| Solvent loss | 5.7% mass loss | | Not observed | | 3.6% mass loss | 1.7% mass | | Not observed | | 5.7% mass |
| | around 100° C. | | | | over broad | loss around | | | | loss around |
| | (assumed water) | | | | range of temp. | 65° C. | | | | 50° C. & |
| | | | | | (25° C. to | | | | | 100° C. |
| | | | | | 200° C.) | | | | | (2 events) |
| Melting point | 149° | C. | 168° | C. | Unclear | 131° | C. | 169° | C. | Unclear, ~150- |
| | | | | | | | | | | 170° C. |
| Number of DSC events | 3 | | 2 | | 4 | 5 | | 3 | | 4 |
| Number of TGA events | 2 | | 1 | | 2 | 3 | | 2 | | 2 |
| DVS | | | | | | | | | | |
| DVS Mass loss on drying | 5% | | 0.1% | | 2% | 1% | | 0.2% | | 5% |
| 0% RH (approximate) | | | | | | | | | | |
| DVS Mass increase at | 4.3% | | 0.06% | | 2.9% | 5.5% | | 0.2% | | 5.1% |
| 50% RH | | | | | | | | | | |
| DVS Mass increase at | 14.7% | | 0.15% | | 11.7% | 19.2% | | 1.4% | | 7.2% |
| 90% RH | | | | | | | | | | |
| Change in XRPD after | Yes | | No | | Yes | Yes | | No | | No |
| DVS | | | | | | | | | | |

1. An oxalate salt of Compound 1.

2. An oxalic acid cocrystal of Compound 1.

3. The oxalic acid cocrystal of claim 2, wherein the cocrystal is Form I that exhibits an XRPD pattern comprising one or more peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.

4. The oxalic acid cocrystal of claim 2, wherein the cocrystal is Form I that exhibits an XRPD pattern comprising three or more peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.

5. The oxalic acid cocrystal of claim 2, wherein the cocrystal is Form I that exhibits an XRPD pattern comprising five or more peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.

6. The oxalic acid cocrystal of claim 2, wherein the cocrystal is Form I that exhibits an XRPD pattern comprising peaks at 10.29±0.2, 12.28±0.2, and 21.78±0.2 degrees two-theta.

7. The oxalic acid cocrystal of claim 2, wherein the cocrystal is Form I that exhibits an XRPD pattern comprising peaks at 6.68±0.2, 8.43±0.2, 10.29±0.2, 10.82±0.2, 12.28±0.2, 14.37±0.2, 20.60±0.2, 21.78±0.2, and 22.45±0.2 degrees two-theta.

8. The oxalic acid cocrystal of claim 2, wherein the Form I exhibits an XRPD pattern substantially similar to FIG. 1A.

9. The oxalic acid cocrystal of claim 3, wherein the Form I exhibits a differential scanning calorimetry (DSC) thermogram comprising an endothermic peak that onsets at 166±5.0° C.

10. The oxalic acid cocrystal of claim 9, wherein the Form I exhibits a differential scanning calorimetry (DSC) thermogram further comprising endothermic peaks that onsets at 180±5.0° C. and 245±5.0° C.

11. The oxalic acid cocrystal of claim 3, wherein the Form I exhibits a DSC thermogram substantially similar to FIG. 1B.

12. The oxalic acid cocrystal of claim 11, wherein the Form I exhibits a weight loss of about 15-25% that onsets at a temperature of 179±5.0° C. by thermogravimetric analysis.

13. The oxalic acid cocrystal of claim 11, wherein the Form I exhibits a weight loss of about 15-25% that onsets at a temperature of 241±5.0° C. by thermogravimetric analysis.

14. The oxalic acid cocrystal of claim 3, wherein the Form I exhibits a thermogravimetric analysis thermogram substantially similar to FIG. 1C.

15. The oxalic acid cocrystal of claim 3, wherein the Form I exhibits a mass loss on drying of about 0.1-0.5% at about 0% relative humidity (RH) according to dynamic vapor sorption (DVS).

16. The oxalic acid cocrystal of claim 3, wherein the Form I exhibits a mass increase for water uptake of about 0.1-0.5% at about 50% RH according to DVS.

17. The oxalic acid cocrystal of claim 3, wherein the Form I exhibits a mass increase for water uptake of about 1-2% at about 90% RH according to DVS.

18. A crystalline freebase of Compound 1.

19. The crystalline freebase of claim 18, wherein the crystalline freebase is Form A that exhibits an XRPD pattern comprising one or more peaks at 6.45±0.2, 8.71±0.2, 11.02±0.2, 11.54±0.2, 12.11±0.2, 12.94±0.2, 14.63±0.2, 15.73±0.2, 16.80±0.2, 17.84±0.2, 18.44±0.2, 19.36±0.2, 19.77±0.2, 20.38±0.2, 20.54±0.2, 20.81±0.2, 21.10±0.2, 22.27±0.2, 27.70±0.2, 22.97±0.2, 24.48±0.2, 25.63±0.2, 25.92±0.2, 27.42±0.2, 27.91±0.2, 29.54±0.2, and 30.12±0.2 degrees two-theta degrees two-theta.

20.-33. (canceled)

34. The crystalline freebase of claim 18, wherein the crystalline freebase is Form C that exhibits an XRPD pattern comprising one or more peaks at 5.48±0.2, 11.20±0.2, 12.55±0.2, 13.06±0.2, 18.05±0.2, 19.41±0.2, 21.48±0.2, 21.58±0.2, 21.87±0.2, 22.75±0.2, 23.65±0.2, 23.99±0.2, 24.79±0.2, 28.05±0.2, and 28.13±0.2 degrees two-theta.

35.-48. (canceled)

49. A pharmaceutical composition comprising the Compound 1 oxalate salt of claim 1, and a pharmaceutically acceptable carrier or excipient.

50. A pharmaceutical composition comprising the Compound 1 oxalic acid cocrystal of claim 2, and a pharmaceutically acceptable carrier or excipient.

51. A method for treating or managing a disease or a disorder mediated by AAK1 activity, comprising administering to a subject in need thereof the pharmaceutical composition of claim 50.

52.-54. (canceled)

55. A method for increasing skeletal muscle tissue growth or regeneration in a subject, comprising administering to the subject the pharmaceutical composition of claim 50.

56.-61. (canceled)

62. A method of treating a muscular dystrophy in a subject in need thereof, comprising administering the pharmaceutical composition of claim 49 to the subject.

63.-67. (canceled)
